SEQUENCE LISTING (140 sequences)

SEQ ID NO: 1 - protein, 133 aa, Homo sapiens [sequence omitted]

SEQ ID NO: 2 - protein, 113 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINT

SEQ ID NO: 3 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 4 - protein, 214 aa, Homo sapiens
AVNGTSQFTCFYNSRANISCVWSQDGALQDTSCQVHAWPDRRRWNQTCELLPVSQASWACNLILGAPESQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 5 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFQRRLEFEARTLSPGHTWEEAPLLTLKQKQEWICLETLTPDTQYEFQVRVKPLQGEFTTWSPWSQPLAFRTKPAALGKDT

SEQ ID NO: 6 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 7 - protein, 75 aa, Homo sapiens
ITCPPPMSVEHADIWVKSYSLYSRERYICNSGFKRKAGTSSLTECVLNKATNVAHWTTPSLKCIRDPALVHQRPA

SEQ ID NO: 8 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 9 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 10 - protein, 76 aa, Homo sapiens
TNDAEDCCLSVTQKPIPGYIVRNFHYLLIKDGCRVPAVVFTTLRGRQLCAPPDQPWVERIIQRLQRTSAKMKRRSS

SEQ ID NO: 11 - protein, 5 aa, synthetic construct
  REGION 1..5: note = Description of Artificial Sequence: Synthetic peptide
GGGGS

SEQ ID NO: 12 - protein, 10 aa, synthetic construct
  REGION 1..10: note = Description of Artificial Sequence: Synthetic peptide
GGGGSGGGGS

SEQ ID NO: 13 - protein, 15 aa, synthetic construct
  REGION 1..15: note = Description of Artificial Sequence: Synthetic peptide
GGGGSGGGGSGGGGS

SEQ ID NO: 14 - protein, 21 aa, synthetic construct
  REGION 1..21: note = Description of Artificial Sequence: Synthetic peptide
  VARIANT 11: note = Ala or Asn
GGGGSGGGGSXGGGGSGGGGS

SEQ ID NO: 15 - protein, 21 aa, synthetic construct
  REGION 1..21: note = Description of Artificial Sequence: Synthetic peptide
  VARIANT 11: note = Ser, Ala or Asn
  VARIANT 16: note = Ala or Asn
GGGGSGGGGSXGGGGXGGGGS

SEQ ID NO: 16 - protein, 32 aa, synthetic construct
  REGION 1..32: note = Description of Artificial Sequence: Synthetic polypeptide
GGGGSGGGGSGGGGSAAGGGGSGGGGSGGGGS

SEQ ID NO: 17 - protein, 6 aa, synthetic construct
  REGION 1..6: note = Description of Artificial Sequence: Synthetic peptide
GPLGVR

SEQ ID NO: 18 - protein, 7 aa, synthetic construct
  REGION 1..7: note = Description of Artificial Sequence: Synthetic peptide
PLGMWSR

SEQ ID NO: 19 - protein, 7 aa, synthetic construct
  REGION 1..7: note = Description of Artificial Sequence: Synthetic peptide
PLGLWAR

SEQ ID NO: 20 - protein, 8 aa, synthetic construct
  REGION 1..8: note = Description of Artificial Sequence: Synthetic peptide
PQGIAGQR

SEQ ID NO: 21 - protein, 6 aa, synthetic construct
  REGION 1..6: note = Description of Artificial Sequence: Synthetic peptide
PLGLAG

SEQ ID NO: 22 - protein, 6 aa, synthetic construct
  REGION 1..6: note = Description of Artificial Sequence: Synthetic peptide
LALGPR

SEQ ID NO: 23 - protein, 10 aa, synthetic construct
  REGION 1..10: note = Description of Artificial Sequence: Synthetic peptide
GGPLGMLSQS

SEQ ID NO: 24 - protein, 9 aa, synthetic construct
  REGION 1..9: note = Description of Artificial Sequence: Synthetic peptide
GGGGRRGGS

SEQ ID NO: 25 - protein, 8 aa, synthetic construct
  REGION 1..8: note = Description of Artificial Sequence: Synthetic peptide
TGRGPSWV

SEQ ID NO: 26 - protein, 8 aa, synthetic construct
  REGION 1..8: note = Description of Artificial Sequence: Synthetic peptide
SARGPSRW

SEQ ID NO: 27 - protein, 8 aa, synthetic construct
  REGION 1..8: note = Description of Artificial Sequence: Synthetic peptide
TARGPSFK

SEQ ID NO: 28 - protein, 7 aa, synthetic construct
  REGION 1..7: note = Description of Artificial Sequence: Synthetic peptide
TARGPSW

SEQ ID NO: 29 - protein, 8 aa, synthetic construct
  REGION 1..8: note = Description of Artificial Sequence: Synthetic peptide
GGWHTGRN

SEQ ID NO: 30 - protein, 8 aa, synthetic construct
  REGION 1..8: note = Description of Artificial Sequence: Synthetic peptide
HTGRSGAL

SEQ ID NO: 31 - protein, 8 aa, synthetic construct
  REGION 1..8: note = Description of Artificial Sequence: Synthetic peptide
PLTGRSGG

SEQ ID NO: 32 - protein, 7 aa, synthetic construct
  REGION 1..7: note = Description of Artificial Sequence: Synthetic peptide
LTGRSGA

SEQ ID NO: 33 - protein, 8 aa, synthetic construct
  REGION 1..8: note = Description of Artificial Sequence: Synthetic peptide
RQARVVNG

SEQ ID NO: 34 - protein, 18 aa, synthetic construct
  REGION 1..18: note = Description of Artificial Sequence: Synthetic peptide
VHMPLGFLGPRQARVVNG

SEQ ID NO: 35 - protein, 16 aa, synthetic construct
  REGION 1..16: note = Description of Artificial Sequence: Synthetic peptide
GGSLSGRSDNHGGGGS

SEQ ID NO: 36 - protein, 42 aa, synthetic construct
  REGION 1..42: note = Description of Artificial Sequence: Synthetic polypeptide
GGGGSGGGGSGGGGSISSGLLSSGGSGGSLSGRSDNHGGGGS

SEQ ID NO: 37 - protein, 467 aa, synthetic construct
  REGION 1..467: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 38 - protein, 448 aa, synthetic construct
  REGION 1..448: note = Description of Artificial Sequence: Synthetic polypeptide
  VARIANT 399: note = Asn or Asp
  VARIANT 442: note = Gln or Glu
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGSGGGGSITCPPPMSVEHADIWVKSYSLYSRERYICNSGFKRKAGTSSLTECVLNKATNVAHWTTPSLKCIRDPALVHQRPAPPSGGGGSGGGGSGGGGSNWVNVISDLKKIEDLIQSMHIDATLYTESDVHPSCKVTAMKCFLLELQVISLESGDASIHDTVEXLIILANNSLSSNGNVTESGCKECEELEEKNIKEFLQSFVHIVXMFINTS

SEQ ID NO: 39 - protein, 465 aa, synthetic construct
  REGION 1..465: note = Description of Artificial Sequence: Synthetic polypeptide
  VARIANT 416: note = Asn or Asp
  VARIANT 459: note = Gln or Glu
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGSGGGGSITCPPPMSVEHADIWVKSYSLYSRERYICNSGFKRKAGTSSLTECVLNKATNVAHWTTPSLKCIRDPALVHQRPAPPSGGGGSGGGGSGGGGSAAGGGGSGGGGSGGGGSNWVNVISDLKKIEDLIQSMHIDATLYTESDVHPSCKVTAMKCFLLELQVISLESGDASIHDTVEXLIILANNSLSSNGNVTESGCKECEELEEKNIKEFLQSFVHIVXMFINTS

SEQ ID NO: 40 - protein, 451 aa, synthetic construct
  REGION 1..451: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGSGGGGSGGGGSGGGGSITCPPPMSVEHADIWVKSYSLYSRERYICNSGFKRKAGTSSLTECVLNKATNVAHWTTPSLKCIRDPALVHQRPAPPS

SEQ ID NO: 41 - protein, 355 aa, synthetic construct
  REGION 1..355: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 42 - protein, 563 aa, synthetic construct
  REGION 1..563: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGSGGGGSGGGGSGGGGSITCPPPMSVEHADIWVKSYSLYSRERYICNSGFKRKAGTSSLTECVLNKATNVAHWTTPSLKCIRDPALVHQRPAPPSGGGGSGGGGSGPLGVRGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 43 - protein, 227 aa, synthetic construct
  REGION 1..227: note = Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - protein, 355 aa, synthetic construct
  REGION 1..355: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGSGGGGSNWVNVISDLKKIEDLIQSMHIDATLYTESDVHPSCKVTAMKCFLLKLQVISLESGDASIHDTVEDLIILANNSLSSNGNVTESGCKECEELEEKNIKEFLQSFVHIVQMFINTS

SEQ ID NO: 45 - protein, 692 aa, synthetic construct
  REGION 1..692: note = Description of Artificial Sequence: Synthetic polypeptide
  VARIANT 643: note = Asn or Asp
  VARIANT 686: note = Gln or Glu
EVQLQQSGAEVKKPGATVKISCTASGFNIKDDYLHWVRQAPGKGLEWIGRIDPANANTKYAPKFQDRVTITADTSTNTAYLELSSLRSEDTAVYYCAARFGYFYGSSFYAVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGSGGGGSITCPPPMSVEHADIWVKSYSLYSRERYICNSGFKRKAGTSSLTECVLNKATNVAHWTTPSLKCIRDPALVHQRPAPPSGGGGSGGGGSGGGGSAAGGGGSGGGGSGGGGSNWVNVISDLKKIEDLIQSMHIDATLYTESDVHPSCKVTAMKCFLLELQVISLESGDASIHDTVEXLIILANNSLSSNGNVTESGCKECEELEEKNIKEFLQSFVHIVXMFINTS

SEQ ID NO: 46 - protein, 690 aa, synthetic construct
  REGION 1..690: note = Description of Artificial Sequence: Synthetic polypeptide
EVQLQQSGAEVKKPGATVKISCTASGFNIKDDYLHWVRQAPGKGLEWIGRIDPANANTKYAPKFQDRVTITADTSTNTAYLELSSLRSEDTAVYYCAARFGYFYGSSFYAVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGSGGGGSGGGGSAAGGGGSGGGGSGGGGSITCPPPMSVEHADIWVKSYSLYSRERYICNSGFKRKAGTSSLTECVLNKATNVAHWTTPSLKCIRDPALVHQRPAPPS

SEQ ID NO: 47 - protein, 694 aa, synthetic construct
  REGION 1..694: note = Description of Artificial Sequence: Synthetic polypeptide
EVQLQQSGAEVKKPGATVKISCTASGFNIKDDYLHWVRQAPGKGLEWIGRIDPANANTKYAPKFQDRVTITADTSTNTAYLELSSLRSEDTAVYYCAARFGYFYGSSFYAVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 48 - protein, 721 aa, synthetic construct
  REGION 1..721: note = Description of Artificial Sequence: Synthetic polypeptide
EVQLQQSGAEVKKPGATVKISCTASGFNIKDDYLHWVRQAPGKGLEWIGRIDPANANTKYAPKFQDRVTITADTSTNTAYLELSSLRSEDTAVYYCAARFGYFYGSSFYAVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSGGGGSGGGGSGGGGSEIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLEISR

SEQ ID NO: 49 - protein, 721 aa, synthetic construct
  REGION 1..721: note = Description of Artificial Sequence: Synthetic polypeptide
EVQLQQSGAEVKKPGATVKISCTASGFNIKDDYLHWVRQAPGKGLEWIGRIDPANANTKYAPKFQDRVTITADTSTNTAYLELSSLRSEDTAVYYCAARFGYFYGSSFYAVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSGGGGSEIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLEISRGGGGSGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 50 - protein, 214 aa, synthetic construct
  REGION 1..214: note = Description of Artificial Sequence: Synthetic polypeptide
DIQMTQSPSSLSASVGDRVTITCRASQDISNYLNWYQQKPDGTVKLLIYYTSRLHSGVPSRFSGSGSGTDYTLTISSLQPEDIATYFCQQGKTLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 362 aa, synthetic construct
  REGION 1..362: note = Description of Artificial Sequence: Synthetic polypeptide
DIQMTQSPSSLSASVGDRVTITCRASQDISNYLNWYQQKPDGTVKLLIYYTSRLHSGVPSRFSGSGSGTDYTLTISSLQPEDIATYFCQQGKTLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSAPASSSTKKTQLQLEHLLLDLQMILNGINNYKNPKLTSMLTAKFAMPKKATELKHLQCLEEALKPLEEVLNLAQSKNFHLRPRDLISEINVIVLELKGSETTFMCEYADETATIVEFLNRWITFSQSIISTLT

SEQ ID NO: 52 - protein, 665 aa, synthetic construct
  REGION 1..665: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSITCPPPMSVEHADIWVKSYSLYSRERYICNSGFKRKAGTSSLTECVLNKATNVAHWTTPSLKCIRDPALVHQRPAPPSGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 53 - protein, 665 aa, synthetic construct
  REGION 1..665: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGSGGGGSGGGGSGGGGSITCPPPMSVEHADIWVKSYSLYSRERYICNSGFKRKAGTSSLTECVLNKATNVAHWTTPSLKCIRDPALVHQRPAPPS

SEQ ID NO: 54 - protein, 680 aa, synthetic construct
  REGION 1..680: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGPLGVRGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 55 - protein, 214 aa, synthetic construct
  REGION 1..214: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 362 aa, synthetic construct
  REGION 1..362: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSAPASSSTKKTQLQLEHLLLDLQMILNGINNYKNPKLTSMLTAKFAMPKKATELKHLQCLEEALKPLEEVLNLAQSKNFHLRPRDLISEINVIVLELKGSETTFMCEYADETATIVEFLNRWITFSQSIISTLT

SEQ ID NO: 57 - protein, 440 aa, synthetic construct
  REGION 1..440: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 58 - protein, 661 aa, synthetic construct
  REGION 1..661: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGSGGGGSITCPPPMSVEHADIWVKSYSLYSRERYICNSGFKRKAGTSSLTECVLNKATNVAHWTTPSLKCIRDPALVHQRPAPPSGGGGSGGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 59 - protein, 661 aa, synthetic construct
  REGION 1..661: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGSGGGGSITCPPPMSVEHADIWVKSYSLYSRERYICNSGFKRKAGTSSLTECVLNKATNVAHWTTPSLKCIRDPALVHQRPAPPSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTS

SEQ ID NO: 60 - protein, 702 aa, synthetic construct
  REGION 1..702: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGPLGVRGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSGGGGSGGGGSGGGGSEIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLEISR

SEQ ID NO: 61 - protein, 702 aa, synthetic construct
  REGION 1..702: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGPLGVRGGGGSGGGGSEIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLEISRGGGGSGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 62 - protein, 568 aa, synthetic construct
  REGION 1..568: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGSGGGGSNWVNVISDLKKIEDLIQSMHIDATLYTESDVHPSCKVTAMKCFLLKLQVISLESGDASIHDTVEELIILANNSLSSNGNVTESGCKECEELEEKNIKEFLQSFVHIVQMFINTS

SEQ ID NO: 63 - protein, 568 aa, synthetic construct
  REGION 1..568: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGSGGGGSNWVNVISDLKKIEDLIQSMHIDATLYTESDVHPSCKVTAMKCFLLKLQVISLESGDASIHDTVEDLIILANNSLSSNGNVTESGCKECEELEEKNIKEFLQSFVHIVQMFINTS

SEQ ID NO: 64 - protein, 569 aa, synthetic construct
  REGION 1..569: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSNWVNVISDLKKIEDLIQSMHIDATLYTESDVHPSCKVTAMKCFLLKLQVISLESGDASIHDTVENLIILANNSLSSNGNVTESGCKECEELEEKNIKEFLQSFVHIVQMFINTS

SEQ ID NO: 65 - protein, 569 aa, synthetic construct
  REGION 1..569: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSNWVNVISDLKKIEDLIQSMHIDATLYTESDVHPSCKVTAMKCFLLKLQVISLESGDASIHDTVEDLIILANNSLSSNGNVTESGCKECEELEEKNIKEFLQSFVHIVQMFINTS

SEQ ID NO: 66 - protein, 706 aa, synthetic construct
  REGION 1..706: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSGGGGSEIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISGGGGSGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 67 - protein, 651 aa, synthetic construct
  REGION 1..651: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSITCPPPMSVEHADIWVKSYSLYSRERYICNSGFKRKAGTSSLTECVLNKATNVAHWTTPSLKCIRGGSGGGGSGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 68 - protein, 668 aa, synthetic construct
  REGION 1..668: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSITCPPPMSVEHADIWVKSYSLYSRERYICNSGFKRKAGTSSLTECVLNKATNVAHWTTPSLKCIRGGSGGGGSGGGSGGGGSAAGGGGSGGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 69 - protein, 678 aa, synthetic construct
  REGION 1..678: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGSGGGGSITCPPPMSVEHADIWVKSYSLYSRERYICNSGFKRKAGTSSLTECVLNKATNVAHWTTPSLKCIRDPALVHQRPAPPSGGGGSGGGGSGGGGSAAGGGGSGGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 70 - protein, 501 aa, synthetic construct
  REGION 1..501: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSVHMPLGFLGPRQARVVNGGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSGGGGSGGGGSGGGGSEIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLEISR

SEQ ID NO: 71 - protein, 501 aa, synthetic construct
  REGION 1..501: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSVHMPLGFLGPRQARVVNGGGGGSGGGGSEIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLEISRGGGGSGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 72 - protein, 501 aa, synthetic construct
  REGION 1..501: note = Description of Artificial Sequence: Synthetic polypeptide
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSVHMPLGFLGPRQARVVNGGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSGGGGSGGGGSGGGGSEIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLEISR

SEQ ID NO: 73 - protein, 501 aa, synthetic construct
  REGION 1..501: note = Description of Artificial Sequence: Synthetic polypeptide
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSVHMPLGFLGPRQARVVNGGGGGSGGGGSEIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLEISRGGGGSGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 74 - protein, 438 aa, synthetic construct
  REGION 1..438: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSITCPPPMSVEHADIWVKSYSLYSRERYICNSGFKRKAGTSSLTECVLNKATNVAHWTTPSLKCIRGGSGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 75 - protein, 438 aa, synthetic construct
  REGION 1..438: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSITCPPPMSVEHADIWVKSYSLYSRERYICNSGFKRKAGTSSLTECVLNKATNVAHWTTPSLKCIRGGSGGGGSGGGSGGGGSNWVNVISDLKKIEDLIQSMHIDATLYTESDVHPSCKVTAMKCFLLKLQVISLESGDASIHDTVEDLIILANNSLSSNGNVTESGCKECEELEEKNIKEFLQSFVHIVQMFINTS

SEQ ID NO: 76 - protein, 438 aa, synthetic construct
  REGION 1..438: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSITCPPPMSVEHADIWVKSYSLYSRERYICNSGFKRKAGTSSLTECVLNKATNVAHWTTPSLKCIRGGSGGGGSGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 77 - protein, 438 aa, synthetic construct
  REGION 1..438: note = Description of Artificial Sequence: Synthetic polypeptide
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSITCPPPMSVEHADIWVKSYSLYSRERYICNSGFKRKAGTSSLTECVLNKATNVAHWTTPSLKCIRGGSGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 78 - protein, 455 aa, synthetic construct
  REGION 1..455: note = Description of Artificial Sequence: Synthetic polypeptide
MGVKVLFALICIAVAEADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSITCPPPMSVEHADIWVKSYSLYSRERYICNSGFKRKAGTSSLTECVLNKATNVAHWTTPSLKCIRGGSGGGGSGGGSGGGGSNWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 79 - protein, 455 aa, synthetic construct
  REGION 1..455: note = Description of Artificial Sequence: Synthetic polypeptide
MGVKVLFALICIAVAEADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSITCPPPMSVEHADIWVKSYSLYSRERYICNSGFKRKAGTSSLTECVLNKATNVAHWTTPSLKCIRGGSGGGGSGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 80 - protein, 503 aa, synthetic construct
  REGION 1..503: note = Description of Artificial Sequence: Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSVHMPLGFLGPRQARVVNGGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSGGGGSGGGGSGGGGSEIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLEISR

SEQ ID NO: 81 - protein, 503 aa, synthetic construct
  REGION 1..503: note = Description of Artificial Sequence: Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSVHMPLGFLGPRQARVVNGGGGGSGGGGSEIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLEISRGGGGSGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 82 - protein, 440 aa, synthetic construct
  REGION 1..440: note = Description of Artificial Sequence: Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSITCPPPMSVEHADIWVKSYSLYSRERYICNSGFKRKAGTSSLTECVLNKATNVAHWTTPSLKCIRGGSGGGGSGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 83 - protein, 459 aa, synthetic construct
  REGION 1..459: note = Description of Artificial Sequence: Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSITCPPPMSVEHADIWVKSYSLYSRERYICNSGFKRKAGTSSLTECVLNKATNVAHWTTPSLKCIRGGSGGGGSGGGSGGGGSAAGGSGGGGSGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 84 - protein, 440 aa, synthetic construct
  REGION 1..440: note = Description of Artificial Sequence: Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSITCPPPMSVEHADIWVKSYSLYSRERYICNSGFKRKAGTSSLTECVLNKATNVAHWTTPSLKCIRGGSGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTS

SEQ ID NO: 85 - protein, 360 aa, synthetic construct
  REGION 1..360: note = Description of Artificial Sequence: Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGSGGGGSGGGSGGGGSNWVNVISDLKKIEDLIQSMHIDATLYTESDVHPSCKVTAMKCFLLKLQVISLESGDASIHDTVENLIILANNSLSSNGNVTESGCKECEELEEKNIKEFLQSFVHIVQMFINTS

SEQ ID NO: 86 - protein, 360 aa, synthetic construct
  REGION 1..360: note = Description of Artificial Sequence: Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGSGGGGSGGGSGGGGSNWVNVISDLKKIEDLIQSMHIDATLYTESDVHPSCKVTAMKCFLLKLQVISLESGDASIHDTVEDLIILANNSLSSNGNVTESGCKECEELEEKNIKEFLQSFVHIVQMFINTS

SEQ ID NO: 87 - protein, 481 aa, synthetic construct
  REGION 1..481: note = Description of Artificial Sequence: Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSVHMPLGFLGPRQARVVNGGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 88 - protein, 214 aa, synthetic construct
  REGION 1..214: note = Description of Artificial Sequence: Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 449 aa, synthetic construct
  REGION 1..449: note = Description of Artificial Sequence: Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 214 aa, synthetic construct
  REGION 1..214: note = Description of Artificial Sequence: Synthetic polypeptide
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 385 aa, synthetic construct
  REGION 1..385: note = Description of Artificial Sequence: Synthetic polypeptide
GHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDERKCCVECPAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 92 - protein, 219 aa, synthetic construct
  REGION 1..219: note = Description of Artificial Sequence: Synthetic polypeptide
DIVMTQAAPSVPVTPGESVSISCRSSKSLLHSNGNTYLYWFLQRPGQSPQVLIYRMSNLASGVPDRFSGSGSGTAFTLRIRRVEAEDVGVYYCMQNLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 444 aa, synthetic construct
  REGION 1..444: note = Description of Artificial Sequence: Synthetic polypeptide
QVQLQQSGPELVKSGASVKMSCKASGNTLKDDHVHWVKQRPGQGLEWIGWIYPGGGRTRYNEKFKGKTTLTADKPSSTVNMLLSSLTSEDSAIYFCTNLVFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 219 aa, synthetic construct
  REGION 1..219: note = Description of Artificial Sequence: Synthetic polypeptide
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 445 aa, synthetic construct
  REGION 1..445: note = Description of Artificial Sequence: Synthetic polypeptide
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 120 aa, synthetic construct
  REGION 1..120: note = Description of Artificial Sequence: Synthetic polypeptide
QVQLVQSGAEVKKPGASVKVSCKASGYSFTGYYMHWVKQSPGQGLEWIGRINPNNGVTLYNQKFKDRVTMTRDTSISTAYMELSRLRSDDTAVYYCARSTMITNYVMDYWGQGTLWTVSS

SEQ ID NO: 97 - protein, 120 aa, synthetic construct
  REGION 1..120: note = Description of Artificial Sequence: Synthetic polypeptide
QVQLVQSGAEVKKPGASVKVSCKASGYSFTGYYMHWVRQAPGQGLEWMGRINPNNGVTLYNQKFKDRVTMTRDTSISTAYMELSRLRSDDTAVYYCARSTMITNYVMDYWGQGTLVTVSS

SEQ ID NO: 98 - protein, 107 aa, synthetic construct
  REGION 1..107: note = Description of Artificial Sequence: Synthetic polypeptide
DIVMTQSPDSLAVSLGERATINCKASQSVSNDVAWYQQKPGQSPKLLISYTSSRYAGVPDRFSGSGSGTDFTLTISSLQAEDVAVYFCQQDYNSPPTFGGGTKLEIK

SEQ ID NO: 99 - protein, 107 aa, synthetic construct
  REGION 1..107: note = Description of Artificial Sequence: Synthetic polypeptide
DIVMTQSPDSLAVSLGERATINCKASQSVSNDVAWYQQKPGQPPKLLIYYTSSRYAGVPDRFSGSGSGTDFTLTISSLQAEDVAVYYCQQDYNSPPTFGGGTKLEIK

SEQ ID NO: 100 - protein, 5 aa, synthetic construct
  REGION 1..5: note = Description of Artificial Sequence: Synthetic peptide
TYWIG

SEQ ID NO: 101 - protein, 17 aa, synthetic construct
  REGION 1..17: note = Description of Artificial Sequence: Synthetic peptide
IIYPGDSDTRYSPSFQG

SEQ ID NO: 102 - protein, 8 aa, synthetic construct
  REGION 1..8: note = Description of Artificial Sequence: Synthetic peptide
GNWNCFDY

SEQ ID NO: 103 - protein, 12 aa, synthetic construct
  REGION 1..12: note = Description of Artificial Sequence: Synthetic peptide
RASQSVSSSYLA

SEQ ID NO: 104 - protein, 7 aa, synthetic construct
  REGION 1..7: note = Description of Artificial Sequence: Synthetic peptide
GASRRAT

SEQ ID NO: 105 - protein, 8 aa, synthetic construct
  REGION 1..8: note = Description of Artificial Sequence: Synthetic peptide
QRYGSSHT

SEQ ID NO: 106 - protein, 8 aa, synthetic construct
  REGION 1..8: note = Description of Artificial Sequence: Synthetic peptide
GNWNSFDY

SEQ ID NO: 107 - protein, 118 aa, synthetic construct
  REGION 1..118: note = Description of Artificial Sequence: Synthetic polypeptide
EVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 108 - protein, 119 aa, synthetic construct
  REGION 1..119: note = Description of Artificial Sequence: Synthetic polypeptide
EIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLEISRTVAAPSVFIFP

SEQ ID NO: 109 - protein, 241 aa, synthetic construct
  REGION 1..241: note = Description of Artificial Sequence: Synthetic polypeptide
EVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSGGGGSGGGGSGGGGSEIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLEISR

SEQ ID NO: 110 - protein, 241 aa, synthetic construct
  REGION 1..241: note = Description of Artificial Sequence: Synthetic polypeptide
EIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLEISRGGGGSGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 111 - protein, 510 aa, synthetic construct
  REGION 1..510: note = Description of Artificial Sequence: Synthetic polypeptide
MGVKVLFALICIAVAEADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSGGGGSGGGGSGGGGSGIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEIS

SEQ ID NO: 112 - protein, 510 aa, synthetic construct
  REGION 1..510: note = Description of Artificial Sequence: Synthetic polypeptide
MGVKVLFALICIAVAEADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSGGGGSGIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISGGGGSGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 113 - protein, 710 aa, synthetic construct
  REGION 1..710: note = Description of Artificial Sequence: Synthetic polypeptide
MGVKVLFALICIAVAEADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGPLGVRGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSGGGGSGGGGSGGGGSGGGGSGGGGSGGGGSPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHW

SEQ ID NO: 114 - protein, 710 aa, synthetic construct
  REGION 1..710: note = Description of Artificial Sequence: Synthetic polypeptide
MGVKVLFALICIAVAEADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGPLGVRGGGGSGGGGSPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGGGGSGGGGSGGGGSGGGGSGGGGSGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 115 - protein, 610 aa, synthetic construct
  REGION 1..610: note = Description of Artificial Sequence: Synthetic polypeptide
MGVKVLFALICIAVAEADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGPLGVRGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSGGGGSGGGGSGGGGSGGGGSGGGGSGGGGSAPENLTLHKLSESQLELNWNNRFLNHCLEHLVQYRTDWDHSWTEQSVDYRHKFSLPSVDGQKRYTFRVRSRFNPLCGSAQHWSEWSHPIHW

SEQ ID NO: 116 - protein, 455 aa, synthetic construct
  REGION 1..455: note = Description of Artificial Sequence: Synthetic polypeptide
MGVKVLFALICIAVAEADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSITCPPPMSVEHADIWVKSYSLYSRERYICNSGFKRKAGTSSLTECVLNKATNVAHWTTPSLKCIRGGSGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTS

SEQ ID NO: 117 - protein, 478 aa, synthetic construct
  REGION 1..478: note = Description of Artificial Sequence: Synthetic polypeptide
MGVKVLFALICIAVAEADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSGGGGSAVNGTSQFTCFYNSRANISCVWSQDGALQDTSCQVHAWPDRRRWNQTCELLPVSQASWACNLILGAPESQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 118 - protein, 455 aa, synthetic construct
  REGION 1..455: note = Description of Artificial Sequence: Synthetic polypeptide
MGVKVLFALICIAVAEADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSITCPPPMSVEHADIWVKSYSLYSRERYICNSGFKRKAGTSSLTECVLNKATNVAHWTTPSLKCIRGGSGGGGSGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 119 - protein, 517 aa, synthetic construct
  REGION 1..517: note = Description of Artificial Sequence: Synthetic polypeptide
MGVKVLFALICIAVAEADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSVHMPLGFLGPRQARVVNGGGGGSGGGGSEIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISGGGGSGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 120 - protein, 518 aa, synthetic construct
  REGION 1..518: note = Description of Artificial Sequence: Synthetic polypeptide
MGVKVLFALICIAVAEADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSGGGGSEIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISGGGGSGGGGSRQARVVNGGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 121 - protein, 373 aa, synthetic construct
  REGION 1..373: note = Description of Artificial Sequence: Synthetic polypeptide
MGVKVLFALICIAVAEADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 122 - protein, 469 aa, synthetic construct
  REGION 1..469: note = Description of Artificial Sequence: Synthetic polypeptide
MGVKVLFALICIAVAEADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSITCPPPMSVEHADIWVKSYSLYSRERYICNSGFKRKAGTSSLTECVLNKATNVAHWTTPSLKCIRGGGGSGGGSGGGGSAAGGGGSGGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 123 - protein, 105 aa, synthetic construct
  REGION 1..105: note = Description of Artificial Sequence: Synthetic polypeptide
EIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLE

SEQ ID NO: 124 - protein, 238 aa, synthetic construct
  REGION 1..238: note = Description of Artificial Sequence: Synthetic polypeptide
EVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSGGGGSGGGGSGGGGSEIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLE

SEQ ID NO: 125 - protein, 11 aa, synthetic construct
  REGION 1..11: note = Description of Artificial Sequence: Synthetic peptide
KASQDVSIAVA

SEQ ID NO: 126 - protein, 7 aa, synthetic construct
  REGION 1..7: note = Description of Artificial Sequence: Synthetic peptide
SASYRYT

SEQ ID NO: 127 - protein, 9 aa, synthetic construct
  REGION 1..9: note = Description of Artificial Sequence: Synthetic peptide
QQHYITPLT

SEQ ID NO: 128 - protein, 5 aa, synthetic construct
  REGION 1..5: note = Description of Artificial Sequence: Synthetic peptide
NYGMN

SEQ ID NO: 129 - protein, 17 aa, synthetic construct
  REGION 1..17: note = Description of Artificial Sequence: Synthetic peptide
WINTYTGEPTYTDDFKG

SEQ ID NO: 130 - protein, 12 aa, synthetic construct
  REGION 1..12: note = Description of Artificial Sequence: Synthetic peptide
GGFGSSYWYFDV

SEQ ID NO: 131 - protein, 10 aa, synthetic construct
  REGION 1..10: note = Description of Artificial Sequence: Synthetic peptide
SASSSVSYMH

SEQ ID NO: 132 - protein, 7 aa, synthetic construct
  REGION 1..7: note = Description of Artificial Sequence: Synthetic peptide
DTSKLAS

SEQ ID NO: 133 - protein, 9 aa, synthetic construct
  REGION 1..9: note = Description of Artificial Sequence: Synthetic peptide
QQWSGYPLT

SEQ ID NO: 134 - protein, 5 aa, synthetic construct
  REGION 1..5: note = Description of Artificial Sequence: Synthetic peptide
GYTMN

SEQ ID NO: 135 - protein, 17 aa, synthetic construct
  REGION 1..17: note = Description of Artificial Sequence: Synthetic peptide
LITPYNGASSYNQKFRG

SEQ ID NO: 136 - protein, 10 aa, synthetic construct
  REGION 1..10: note = Description of Artificial Sequence: Synthetic peptide
GGYDGRGFDY

SEQ ID NO: 137 - DNA, 1566 bp, synthetic construct
  misc_feature 1..1566: note = Description of Artificial Sequence: Synthetic polynucleotide
  source 1..1566: mol_type = other DNA
ctgggcagcgctcgcccggggagtccagcggtgtcctgtggagctgccgccatggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgaaactcggggaaaccagcccagctaagtccggagtgaaggagcctctctgctttagctaaagacgactgagaagaggtgcaaggaagcgggctccaggagcaagctcaccaggcctctcagaagtcccagcaggatctcacggactgccgggtcggcgcctcctgcgcgagggagcaggttctccgcattcccatgggcaccacctgcctgcctgtcgtgccttggacccagggcccagcttcccaggagagaccaaaggcttctgagcaggatttttatttcattacagtgtgagctgcctggaatacatgtggtaatgaaataaaaaccctgccccgaatcttccgtccctcatcctaactttcagttcacagagaaaagtgacatacccaaagctctctgtcaattacaaggcttctcctggcgtgggagacgtctacagggaagacaccagcgtttgggcttctaaccaccctgtctccagctgctctgcacacatggacagggacctgggaaaggtgggagagatgctgagcccagcgaatcctctccattgaaggattcaggaagaagaaaactcaactcagtgccattttacgaatatatgcgtttatatttatacttccttgtctattatatctatacattatatattatttgtattttgacattgtaccttgtataaacaaaataaaacatctattttcaata

SEQ ID NO: 138 - protein, 12 aa, synthetic construct
  REGION 1..12: note = Description of Artificial Sequence: Synthetic peptide
GSAGSAAGSGEF

SEQ ID NO: 139 - protein, 42 aa, synthetic construct
  REGION 1..42: note = Description of Artificial Sequence: Synthetic polypeptide
  VARIANT 1..15: note = This region may encompass 1-3 "Gly Gly Gly Gly Ser" repeating units
  VARIANT 28..42: note = This region may encompass 1-3 "Gly Gly Gly Gly Ser" repeating units
GGGGSGGGGSGGGGSGSAGSAAGSGEFGGGGSGGGGSGGGGS

SEQ ID NO: 140 - protein, 32 aa, synthetic construct; identical to SEQ ID NO: 16
  REGION 1..32: note = Description of Artificial Sequence: Synthetic polypeptide
  VARIANT 1..15: note = This region may encompass 2-3 "Gly Gly Gly Gly Ser" repeating units
  VARIANT 18..32: note = This region may encompass 2-3 "Gly Gly Gly Gly Ser" repeating units

(19) United States
(12) Patent Application Publication
(10) Pub. No.: US 2024/0067739 A1
(43) Pub. Date: Feb. 29, 2024

(54) NOVEL IL-15 PRODRUGS AND METHODS OF USE THEREOF

(71) Applicant: ASKGENE PHARMA, INC., Camarillo, CA (US)

(72) Inventors: Yuefeng LU, Moorpark, CA (US); Chunxiao YU, Santa Barbara, CA (US); Jian-Feng LU, Oak Park, CA (US)

(73) Assignee: ASKGENE PHARMA, INC., Camarillo, CA (US)

(21) Appl. No.: 18/490,268

(22) Filed: Oct. 19, 2023

Related U.S. Application Data
(62) Division of application No. 16/900,677, filed on Jun. 12, 2020, now Pat. No. 11,845,801.
(60) Provisional application No. 62/860,635, filed on Jun. 12, 2019.
(60) Provisional application No. 62/888,444, filed on Aug. 17, 2019.
(60) Provisional application No. 62/891,190, filed on Aug. 23, 2019.
(60) Provisional application No. 62/959,973, filed on Jan. 11, 2020.
(60) Provisional application No. 63/029,473, filed on May 23, 2020.

(51) Int. Cl. C07K 16/28; C07K 14/81

(52) U.S. Cl. CPC C07K 16/2866; C07K 14/811; C07K 14/8146; C07K 2317/51; C07K 2317/515; C07K 2317/55; C07K 2317/565; C12Y 304/21109; C12Y 304/24024; C12Y 304/24035

(57) ABSTRACT

Provided herein are IL-15 cytokine prodrugs and methods of making and using thereof.

### CROSS-REFERENCE TO RELATED APPLICATIONS

This application is a division of U.S. patent application Ser. No. 16/900,677, filed on Jun. 12, 2020, which claims priority from U.S. Provisional Applications 62/860,635, filed Jun. 12, 2019; 62/888,444, filed Aug. 17, 2019; 62/891,190, filed Aug. 23, 2019; 62/959,973, filed Jan. 11, 2020; and 63/029,473, filed May 23, 2020. The disclosures of the aforementioned priority applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jun. 8, 2020, is named 025471_US004_SL.txt and is 359,580 bytes in size.

### BACKGROUND OF THE INVENTION

Interleukin-15 (IL-15) is a cytokine with structural similarities to IL-2. IL-15 is secreted by mononuclear phagocytes and other immune cells following viral infection. IL-15 induces proliferation of natural killer (NK) and other cells of the immune system and is involved in the killing of virally infected cells and cancer cells. Like IL-2, IL-15 binds to the IL-2 receptor (IL-2R) β/γ complex, the intermediate affinity receptor, with a K_{D} of about 1 nM (Giri et al., EMBO J. (1994) 13:2822-30). IL-15 binds to IL-15 receptor (IL-15R) α with a much higher affinity (K_{D}=˜0.05 nM). IL-15Rα can associate with the IL-2β/γ complex to form an IL-15-specific, functional high-affinity receptor (αβγ) (Minami et al., Annu Rev Immunol. (1993) 11:245-67; Giri et al., J Leukoc Biol. (1995) 5745:763-6; and Lehours et al., Eur Cytokine Netw. (2000) 11:207-15).

The extracellular region of IL-15Rα contains a Sushi domain, which is a common motif in protein-protein interaction. It has been shown that the IL-15Rα N-terminal fragment with the first 65 amino acids is partially active, while the fragment with the first 85 amino acids is fully functional (Wei et al., J Immunol. (2001) 167(1):277-82).

Mutations of IL-15 have been made to study IL-15's interaction with its receptors. D8 and Q108, for example, have been shown to be involved in IL-15's binding to the IL-2Rβ and γ subunits, respectively (Pettit et al., J Biol Chem. (1997) 272: 2312-18). Additional mutations of IL-15 have been disclosed (U.S. Pat. 7,858,081), including those at residues L45, Q48, S51, L52, E64, N65, I68 and L69 of IL-15, which are involved in IL-15 binding to IL-15Rγ or IL-2Rβ. IL-15 muteins with mutation E64K, N65K, N65D, L66D, L66E, I67D, I67E or I68D have been shown to have reduced biological activities in cell-based assays (Zhu et al., J Immnol. (2009) 183(6):3598; and WO2005/085282A1). Mutations targeting IL-15 interaction with IL-15Rα have also been reported. For example, E46, V49, L45, S51, and L52 have been shown to be involved in IL-15Rα binding (Bernard et al., J Biol Chem. (2004) 279:24313-22). E46 appears to be particularly crucial because replacement of its acidic side chain with a basic one (E46K) results in a complete loss of IL-15 binding to IL-15Rα and bioactivity.

Unfortunately, the adverse effects of the current IL-15 drug candidates are significant, limiting the dosing amounts of such drugs. In addition, the activation of T, NK, and other immune cells by these drug candidates are not site specific. Further, there appears to be “PK sinkers” for IL-15 muteins even though their affinities for the IL-15/2 receptors have been significantly reduced. There are also numerous difficulties in the production of IL-15-based protein therapeutics. All of the above underscore the need to develop improved IL-15-based therapeutics.

### SUMMARY OF THE INVENTION

The present disclosure provides a prodrug comprising an IL-15 cytokine moiety (A), a masking moiety (M), a carrier moiety (C), and a Sushi domain (S), wherein the masking moiety binds to the IL-15 cytokine moiety and inhibits a biological activity of the IL-15 cytokine moiety, the masking moiety is fused to the carrier moiety, the Sushi domain is fused to the carrier moiety, and the IL-15 cytokine moiety is fused to the Sushi domain. In some embodiments, the masking moiety is fused to the carrier moiety through a first peptide linker, the Sushi domain is fused to the carrier moiety through a second peptide linker, and the IL-15 cytokine moiety is fused to the Sushi domain through a third peptide linker, and wherein at least one of the three peptide linkers (e.g., one, two, or three) is cleavable. In some embodiments, at least one of the three peptide linkers (e.g., one, two, or three) is noncleavable. In some embodiments, all of the peptide linkers are noncleavable. In particular embodiments, the third peptide linker is at least 15, 20, 25, or 30 amino acids in length (e.g., 15-50 or 15-100 amino acids in length), optionally wherein the third peptide linker comprises SEQ ID NO: 139 or 140.

The present disclosure also provides a prodrug comprising an IL-15 cytokine moiety (A), a masking moiety (M), a carrier moiety (C), and a Sushi domain (S), wherein the masking moiety binds to the IL-15 cytokine moiety and inhibits a biological activity of the IL-15 cytokine moiety, the IL-15 cytokine moiety is fused to the carrier moiety, the Sushi domain is fused to the carrier moiety, and the masking moiety is fused to the Sushi domain. In some embodiments, the IL-15 cytokine moiety is fused to the carrier moiety through a first peptide linker, -the Sushi domain is fused to the carrier moiety through a second peptide linker, and the masking moiety is fused to the Sushi domain through a third peptide linker, and wherein at least one of the three peptide linkers (e.g., one, two, or three) is cleavable. In some embodiments, at least one of the three peptide linkers (e.g., one, two, or three) is noncleavable. In some embodiments, all of the three peptide linkers are noncleavable.

The present disclosure further provides a prodrug comprising an IL-15 cytokine moiety (A), a masking moiety (M), a carrier moiety (C), and a Sushi domain (S), wherein the masking moiety binds to the IL-15 cytokine moiety and inhibits a biological activity of the IL-15 cytokine moiety, the masking moiety is fused to the carrier moiety, the IL-15 moiety is fused to the carrier moiety, and the Sushi domain is fused to the IL-15 moiety. In some embodiments, the masking moiety is fused to the carrier moiety through a first peptide linker, the IL-15 moiety is fused to the carrier moiety through a second peptide linker, and the Sushi domain is fused to the IL-15 moiety through a third peptide linker, and wherein at least one of the three peptide linkers (e.g., one, two, or three) is cleavable. In some embodiments, at least one of the three peptide linkers (e.g., one, two, or three) is noncleavable. In some embodiments, all of the peptide linkers are noncleavable. In particular embodiments, the third peptide linker is at least 15, 20, 25, or 30 amino acids in length (e.g., 15-50 or 15-100 amino acids in length), optionally wherein the third peptide linker comprises SEQ ID NO: 139 or 140.

The present disclosure also provides a prodrug comprising an IL-15 cytokine moiety (A), a masking moiety (M), a carrier moiety (C), and a Sushi domain (S), wherein the masking moiety binds to the IL-15 cytokine moiety and inhibits a biological activity of the IL-15 cytokine moiety, the IL-15 cytokine moiety is fused to the carrier moiety, the masking moiety is fused to the carrier moiety, and the Sushi domain is fused to the masking moiety. In some embodiments, the IL-15 cytokine moiety is fused to the carrier moiety through a first peptide linker, the masking moiety is fused to the carrier moiety through a second peptide linker, and the Sushi domain is fused to the masking moiety through a third peptide linker, and wherein at least one of the three peptide linkers (e.g., one, two, or three) is cleavable. In some embodiments, at least one of the three peptide linkers (e.g., one, two, or three) is noncleavable. In some embodiments, all of the three peptide linkers are noncleavable.

The present disclosure also provides a prodrug comprising an IL-15 cytokine moiety (A), a masking moiety (M), a carrier moiety (C), and a Sushi domain (S), wherein the masking moiety binds to the IL-15 cytokine moiety and inhibits a biological activity of the IL-15 cytokine moiety, the IL-15 cytokine moiety is fused to the carrier moiety, the masking moiety is fused to the IL-15 moiety, and the Sushi domain is fused to the carrier moiety. In some embodiments, the IL-15 cytokine moiety is fused to the carrier moiety through a first peptide linker, the masking moiety is fused to the IL-15 moiety through a second peptide linker, and the Sushi domain is fused to the carrier through a third peptide linker, and wherein at least one of the three peptide linkers (e.g., one, two, or three) is cleavable. In some embodiments, at least one of the three peptide linkers (e.g., one, two, or three) is noncleavable. In some embodiments, all of the three peptide linkers are noncleavable.

The present disclosure also provides a prodrug comprising an IL-15 cytokine moiety (A), a masking moiety (M), a carrier moiety (C), and a Sushi domain (S), wherein the masking moiety binds to the IL-15 cytokine moiety and inhibits a biological activity of the IL-15 cytokine moiety, the masking moiety is fused to the carrier moiety, the IL-15 moiety is fused to the masking moiety, and the Sushi domain is fused to the carrier moiety. In some embodiments, the masking moiety is fused to the carrier moiety through a first peptide linker, the IL-15 moiety is fused to the masking moiety through a second peptide linker, and the Sushi domain is fused to the carrier through a third peptide linker, and wherein at least one of the three peptide linkers (e.g., one, two, or three) is cleavable. In some embodiments, at least one of the three peptide linkers (e.g., one, two, or three) is noncleavable. In some embodiments, all of the three peptide linkers are noncleavable.

In some embodiments, the masking moiety comprises an extracellular domain (ECD) of a receptor of the IL-15 cytokine moiety. For example, the masking moiety comprises an ECD of human IL-2Rμ or a functional analog thereof, and/or an ECD of human IL-2Rγ or a functional analog thereof. In particular embodiments, the ECD of human IL-2Rγ or a functional analog thereof comprises SEQ ID NO: 6, or an amino acid sequence at least 90% identical thereto. In other particular embodiments, the ECD of human IL-2Rμ or a functional analog thereof comprises SEQ ID NO: 3, 4, or 5, or an amino acid sequence at least 90% thereto. In other embodiments, the masking moiety comprises an antibody fragment that binds to the IL-15 cytokine moiety.

The present disclosure further provides a prodrug comprising an IL-15 cytokine moiety (A), a masking moiety (M), a carrier moiety (C), and optionally a Sushi domain (S), wherein the masking moiety comprises an antibody fragment that binds to the IL-15 cytokine moiety and inhibits a biological activity of the IL-15 cytokine moiety, and the masking moiety is fused to the carrier moiety, to the IL-15 cytokine moiety, or to the Sushi domain optionally through a peptide linker.

In some embodiments, the antibody fragment in the prodrug is an ScFv or Fab comprising heavy chain CDR1-3 and light chain CDR1-3 of an anti-IL-15 antibody selected from 146B7, 146H5, 404E4, and 404A8. For example, the antibody fragment comprises heavy chain CDR (HCDR) 1 comprising SEQ ID NO: 100, HCDR2 comprising SEQ ID NO: 101, HCDR3 comprising SEQ ID NO: 102 or 106, light chain CDR (LCDR) 1 comprising SEQ ID NO: 103, LCDR2 comprising SEQ ID NO: 104, and LCDR3 comprising SEQ ID NO: 105. In particular embodiments, the antibody fragment comprises (i) a heavy chain variable domain comprising SEQ ID NO: 107 or an amino acid sequence at least 95% identical thereto, and a light chain variable domain comprising SEQ ID NO: 108 or 123 or an amino acid sequence at least 95% identical thereto; (ii) SEQ ID NO: 109; (iii) SEQ ID NO: 110; or (iv) SEQ ID NO: 124. In certain embodiments, the Cys residue of the heavy chain CDR3 (SEQ ID NO: 102) is mutated to Ser, Thr, Met, Ala, Gly, Asn or Gln.

In some embodiments, the masking moiety does not interfere with or has minimum impact on the binding of the IL-15 cytokine moiety to IL-15Rα.

In some embodiments, the IL-15 cytokine moiety is a human IL-15 polypeptide comprising SEQ ID NO: 2 or a mutein thereof. In particular embodiments, the human IL-15 polypeptide comprises one or more mutations selected from N1A, N1D, N4A, N4D, I6T, S7A, D8A, D8T, D8E, D8N, K10A, K10D, K11A, K11D, E46, V49, L45, S51, L52, D61A, D61N, T62L, T62A, E64A, E64L, E64K, E64Q, N65A, N65L, N65D, L66D, L66E, I67D, I67E, I68S, I68E, L69S, L69E, N72A, N72D, V63E, V63D, L66E, L66D, 167E, I67D, Q108E, N112A, N1D/D61N, N1D/E64Q, N4D/D61N, N4D/E64Q, D8N/D61N, D8N/E64Q, D61N/E64Q, E64Q/Q108E, N1D/N4D/D8N, D61N/E64Q/N65D, N1D/D61N/E64Q, N1D/Q108E, N1D/D61N/E64Q/Q108E, N4D/D61N/E64Q/Q108E, and D30N/E64Q/N65D relative to SEQ ID NO: 2.

In some embodiments, the carrier moiety is a PEG molecule, an albumin, an albumin fragment, an antibody Fc domain, or an antibody or an antigen-binding fragment thereof. In further embodiments, the carrier moiety is an antibody Fc domain or an antibody comprising mutations L234A and L235A (“LALA”) (EU numbering). In some embodiments, the carrier moiety is an antibody Fc domain or an antibody comprising knobs-into-holes mutations, and wherein the IL-15 cytokine moiety and the masking moiety are fused to different polypeptide chains of the antibody Fc domain or to the different heavy chains of the antibody. In certain embodiments, the knobs-into-holes mutations comprise a T366Y “knob” mutation on a polypeptide chain of the Fc domain or a heavy chain of the antibody, and a Y407T “hole” mutation in the other polypeptide of the Fc domain or the other heavy chain of the antibody, or the knobs-into-holes mutations comprise Y349C and/or T366W mutations in the CH3 domain of the “knob chain” and E356C, T366S, L368A, and/or Y407V mutations in the CH3 domain of the “hole chain” (EU numbering). In certain embodiments, the carrier moiety is an IgG₄ Fc domain, and wherein said first polypeptide comprises an amino acid sequence at least 99% identical as one shown in SEQ ID NOs: 80, 81 or 87, and said second polypeptide chain comprises an amino acid sequence at least 99% identical as one selected from SEQ ID NOs: 82-86.

In some embodiments, the carrier moiety is an anti-PD-1 antibody comprising a light chain having an amino acid sequence at least 99% identical to SEQ ID NO: 55 or 56; a first heavy chain having an amino acid sequence at least 99% identical to SEQ ID NO: 54, 60, or 61; and a second heavy chain having an amino acid sequence at least 99% identical to SEQ ID NO: 52, 53, 58, 59, 62, 63, or 69. In further embodiments, the carrier moiety is an anti-PD-1 antibody comprising a light chain having an amino acid sequence at least 99% identical to SEQ ID NO: 55; a first heavy chain having an amino acid sequence at least 99% identical to SEQ ID NO: 66; and a second heavy chain having an amino acid sequence at least 99% identical to SEQ ID NO: 64, 65, 67, or 68.

In some embodiments, the carrier moiety is an anti-PD-L1 antibody comprising a light chain having an amino acid sequence at least 99% identical to SEQ ID NO: 50 or 51; a first heavy chain having an amino acid at least 99% identical to SEQ ID NO: 47, 48 or 49; and a second heavy chain having an amino acid sequence at least 99% identical to SEQ ID NO: 45 or 46.

In some embodiments, the carrier moiety is an antibody or an antigen-binding fragment thereof that specifically binds to one or more antigens selected from PD-1, PD-L1, CTLA-4, LAG-3, TIM-3, and TIGIT.

In some embodiments, the carrier moiety is an antibody Fc domain or an antibody, and the prodrug comprises the following polypeptide pairs (from N-terminus to C-terminus): C1-A and C2-S-M, A-C1 and M-S-C2, C1-S-A and C2-M, C1-A-S and C2-M, S-A-C1 and M-C2, or A-S-C1 and M-C2; and wherein C1 and C2 are the first and second polypeptide chains, respectively, of the Fc domain, or are the first and second heavy chains, respectively, of the antibody; and “-” is a direct peptidyl bond or a peptide linker.

In some embodiments, the Sushi domain comprises SEQ ID NO: 7 or 9, or an amino acid sequence at least 90% identical thereto.

In some embodiments, at least one of the first, second, and third peptide linkers is a noncleavable peptide linker, optionally selected from SEQ ID NOs: 11-16.

In some embodiments, at least one of the first, second, and third peptide linkers is a cleavable peptide linker comprising a substrate sequence of urokinase-type plasminogen activator (uPA), matriptase, matrix metallopeptidase (MMP) 2, or MMP9. For example, the cleavable peptide linker comprises substrate sequences of (i) both uPA and MMP2, (ii) both uPA and MMP9, (iii) uPA, MMP2 and MMP9, or (iv) MMP2 and matriptase. In particular embodiments, the cleavable peptide linker comprises an amino acid sequence selected from SEQ ID NOs: 17-36. The cleavable peptide linker is cleavable by one or more proteases located at a tumor site or its surrounding environment, and the cleavage leads to activation of the prodrug at the tumor site or surrounding environment.

In other aspects, the present disclosure provides a pharmaceutical composition comprising the present prodrug and a pharmaceutically acceptable excipient; a polynucleotide or polynucleotides encoding the present prodrug; an expression vector or vectors comprising the polynucleotide or polynucleotides; and a host cell comprising the vector(s). In some embodiments, the gene(s) encoding uPA, matriptase, MMP-2, and/or MMP-9 are knocked out in the host cell.

Also provided is a method of making the present prodrug, comprising culturing the host cell under conditions that allow expression of the prodrug, wherein the host cell is a mammalian cell, and isolating the prodrug.

In another aspect, the present disclosure provides a method of treating a cancer or an infectious disease or stimulating the immune system in a patient in need thereof, comprising administering to the patient a therapeutically effective amount of the pharmaceutical composition comprising the present prodrug. The patient may have, for example, HIV infection, or a cancer selected from the group consisting of breast cancer, lung cancer, pancreatic cancer, esophageal cancer, medullary thyroid cancer, ovarian cancer, uterine cancer, prostate cancer, testicular cancer, colorectal cancer, and stomach cancer. Also provided are IL-15 prodrugs for use in such treatment, and the use of IL-15 prodrugs for the manufacture of a medicament for such treatment.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTIONS OF THE DRAWINGS

FIGS. 1A-C are schematic illustrations of IL-15 prodrugs with an Fc domain as the carrier moiety. FIG. 1A shows an IL-15Rα Sushi domain polypeptide fused to the C-terminus of one Fc polypeptide, optionally through a noncleavable peptide linker. An IL-15 polypeptide is fused to the C-terminus of the Sushi domain, optionally through a noncleavable linker. A masking moiety is fused to the C-terminus of the other Fc polypeptide through a cleavable linker. FIG. 1B shows an IL-15 polypeptide fused to the C-terminus of one Fc polypeptide, optionally through a noncleavable peptide linker. An IL-15Rα Sushi domain is fused to the C-terminus of the IL-15 polypeptide, optionally through a noncleavable linker. A masking moiety is fused to the C-terminus of the other Fc polypeptide through a cleavable linker. FIG. 1C shows an IL-15 polypeptide fused to the C-terminus of one Fc polypeptide, optionally through a noncleavable peptide linker. An IL-15Rα Sushi domain is fused to the C-terminus of the other Fc polypeptide, optionally through a noncleavable linker. A masking moiety is fused to the C-terminus of the Sushi domain through a cleavable linker. In all three configurations, the Fc domain contains a knobs-into-holes mutation.

FIGS. 2A-C are schematic illustrations IL-15 prodrugs with an Fc domain as the carrier moiety. FIG. 2A shows an IL-15Rα Sushi domain is fused to the N-terminus of one Fc polypeptide, optionally through a noncleavable linker. An IL-15 polypeptide is fused to the N-terminus of the Sushi domain, optionally through a noncleavable peptide linker. A masking moiety is fused to the N-terminus of the other Fc polypeptide through a cleavable linker. FIG. 2B shows an IL-15 polypeptide fused to the N-terminus of one Fc polypeptide, optionally through a noncleavable linker. An IL-15Rα Sushi domain polypeptide is fused to the N-terminus of the IL-15 polypeptide, optionally through a noncleavable peptide linker. A masking moiety is fused to the N-terminus of the other Fc polypeptide through a cleavable linker. FIG. 2C shows an IL-15 polypeptide fused to the N-terminus of one Fc polypeptide, optionally through a noncleavable peptide linker. An IL-15Rα Sushi domain is fused to the N-terminus of the other Fc polypeptide, optionally through a noncleavable linker. A masking moiety is fused to the N-terminus of the Sushi domain through a cleavable linker. In all three configurations, the Fc domain contains a knobs-into-holes mutation.

FIGS. 3A-C are schematic illustrations of IL-15 prodrugs with an antibody (having two antigen-binding sites) as the carrier moiety. FIG. 3A shows an IL-15 polypeptide fused to the C-terminus of one of the heavy chains of the antibody, optionally through a noncleavable peptide linker. An IL-15Rα Sushi domain is fused to the C-terminus of the IL-15 polypeptide, optionally through a noncleavable linker. A masking moiety is fused to the C-terminus of the other heavy chain of the antibody through a cleavable linker. FIG. 3B shows an IL-15Rα Sushi domain polypeptide fused to the C-terminus of one of the heavy chains of the antibody, optionally through a noncleavable peptide linker. An IL-15 polypeptide is fused to the C-terminus of the Sushi domain, optionally through a noncleavable linker. A masking moiety is fused to the C-terminus of the other heavy chain of the antibody through a cleavable linker. FIG. 3C shows an IL-15 polypeptide fused to the C-terminus of one of the heavy chains of the antibody, optionally through a noncleavable peptide linker. An IL-15Rα Sushi domain is fused to the C-terminus of the other heavy chain of the antibody, optionally through a noncleavable linker. A masking moiety is fused to the C-terminus of the Sushi domain through a cleavable linker. In all three FIGs, the antibody contains a knobs-into-holes mutation.

FIGS. 4A and 4B are schematic illustrations of IL-15 prodrugs with an antibody as the carrier moiety. The antibody has a single antigen-binding site. FIG. 4A shows an IL-15 polypeptide fused to the C-terminus of one of the heavy chains of the antibody, optionally through a noncleavable peptide linker. An IL-15Rα Sushi domain is fused to the C-terminus of the IL-15 polypeptide, optionally through a noncleavable linker. A masking moiety is fused to the C-terminus of the other heavy chain of the antibody through a cleavable linker. FIG. 4B shows an IL-15Rα Sushi domain polypeptide fused to the C-terminus of one of the heavy chains of the antibody, optionally through a noncleavable peptide linker. An IL-15 polypeptide is fused to the C-terminus of the Sushi domain, optionally through a noncleavable linker. A masking moiety is fused to the C-terminus of the other heavy chain of the antibody through a cleavable linker. In both configurations, the antibody contains a knobs-into-holes mutation and the masking moiety is on the same polypeptide chain as the heavy chain variable region of the antibody.

FIGS. 5A and 5B are schematic illustrations of IL-15 prodrugs with an antibody as the carrier moiety. The antibody has a single antigen-binding moiety. FIG. 5A shows an IL-15 polypeptide fused to the C-terminus of one of the heavy chains of the antibody, optionally through a noncleavable peptide linker. An IL-15Rα Sushi domain is fused to the C-terminus of the IL-15 polypeptide, optionally through a noncleavable linker. A masking moiety is fused to the C-terminus of the other heavy chain of the antibody through a cleavable linker. FIG. 5B shows an IL-15Rα Sushi domain polypeptide fused to the C-terminus of one of the heavy chains of the antibody, optionally through a noncleavable peptide linker. An IL-15 polypeptide is fused to the C-terminus of the Sushi domain, optionally through a noncleavable linker. A masking moiety is fused to the C-terminus of the other heavy chain of the antibody through a cleavable linker. In both configurations, the antibody contains a knobs-into-holes mutation, and the IL-15 polypeptide and Sushi domain are on the same polypeptide chain as the heavy chain variable region of the antibody.

FIG. 6A shows the sequence information for the Fc-IL-15 prodrugs (JR3.68.1, JR3.68.2 and JR3.68.3) and the control molecules (Fc-IL-15 fusion polypeptides, JR3.68.4 and JR3.68.5).

FIG. 6B illustrates the structures of the molecules of FIG. 6A. All of the molecules have an Fc domain as the carrier moiety. In JR3.68.1, the Sushi domain is fused to the C-terminus of one Fc polypeptide, through a noncleavable linker. The IL-15 polypeptide is fused to the C-terminus of the Sushi domain via a noncleavable linker. A masking moiety is fused to the C-terminus of the other Fc polypeptide via a cleavable linker. In JR3.68.2, an IL-15 polypeptide is fused to the C-terminus of one Fc domain polypeptide via a noncleavable linker. The Sushi domain is fused to the C-terminus of the IL-15 polypeptide through a noncleavable linker. A masking moiety is fused to the C-terminus of the other Fc polypeptide via a cleavable linker. In JR3.68.3, an IL-15 polypeptide is fused to the C-terminus of one Fc polypeptide via a noncleavable linker. The Sushi domain is fused to the C-terminus of the other Fc polypeptide via a noncleavable linker. A masking moiety is fused to the C-terminus of the Sushi domain via a cleavable linker. JR3.68.4 and JR3.68.5 are the activated forms (where the masking moiety was not designed in the constructs) of JR3.68.1 and JR3.68.2, respectively.

FIGS. 7A and 7B are photographs of SDS-PAGE gels analyzing the activatable fusion polypeptides prior to and after activation, as shown in FIG. 6B.

FIGS. 8A-C are graphs show the SEC-HPLC analysis of the Fc-IL-15/Sushi fusion protein samples JR3.68.1, JR3.68.2 and JR3.68.3, respectively, purified by Protein A columns.

FIGS. 9A-C illustrate the cell-based activities of the activatable Fc-IL-15 fusion polypeptides JR3.68.1, JR3.68.2, and JR3.68.3, respectively, before and after activation. In all three figures, IL-15 was used as a positive control.

FIG. 10A is a table shows the sequence information for the antibody-IL-15 fusion polypeptides JR3.74.1 and JR3.74.2 (without mask) and activatable antibody-IL-15 fusion polypeptides JR3.73.2 and JR3.73.4.

FIG. 10B illustrates the structures of the molecules of FIG. 10A.

FIGS. 11A and 11B are graphs shows the SEC-HPLC analysis of JR3.74.1, JR3.74.2, JR3.73.2, and JR3.73.4 samples purified by Protein A columns.

FIG. 11C is a graph showing the results of the CTLL2 proliferation assay on the prodrug samples prior to and after activation with protease treatment.

FIGS. 12A and 12B show the NK92 proliferation assay results of the IL-15 prodrugs masked by an scFv (scFv1 or scFv2) derived from the anti-IL-15 antibody 146B7. FIG. 12A shows the sequence information of the activatable IL-15 fusion proteins. FIG. 12B shows the results of the NK92 proliferation assay. Reference X1: XmAb®24306, which is an IL-15/IL-15-receptor alpha complex fused to a XmAb Fc domain (IL-15/IL-15Rα-Fc). Fc-IL-15*: activatable IL-15 fusion protein with an IL-2Rβ extracellular domain (ECD) as the masking moiety. Fc-IL-15: an Fc-IL-15 fusion protein without the masking moiety. RLU: relative luminescence units.

FIGS. 13A and 13B show the NK92 cell-based activities of the activatable IL-15 fusion proteins prior to and after activation. FIG. 13A shows the NK92 cell-based activities of IL-15 fusion proteins comprising wild type IL-15. FIG. 13B shows the NK92 cell-based activities of IL-15 fusion polypeptides comprising an IL-15 mutein with an N65D mutation. Reference X1: XmAb® 24306, which is an IL-15/IL-15-receptor alpha complex fused to a XmAb Fc domain (IL-15/IL-15Rα-Fc). LUC: signal in luminescence units. Act: activated.

FIG. 14A is a table showing the sequence information for activatable IL-15 fusion proteins.

FIGS. 14B-D show the NK92 proliferation assay results of the activatable IL-15 fusion proteins before and after activation. FIG. 14B shows the results of wild type IL-15 masked by an IL-2Rβ ECD and an IL-2Rγ ECD. FIG. 14C shows the results of IL-15 mutein Q108E masked with an IL-2Rβ ECD and an IL-2Rγ ECD. FIG. 14D shows the results of the activatable Fc-IL-15 fusion protein without a Sushi domain (JR2.145.1) and one with a longer linker between the Sushi domain and the IL-15 polypeptide moiety (JR2.145.2). Reference X1: XmAb®24306, which is an IL-15/IL-15-receptor alpha complex fused to a XmAb Fc domain (IL-15/IL-15Rα-Fc). Reference X2: is a PD-1 antibody-IL-15 mutein fusion protein without a Sushi domain.

### DETAILED DESCRIPTION OF THE INVENTION

As used herein and in the appended claims, the singular forms “a,” “or,” and “the” include plural referents unless the context clearly dictates otherwise.

Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z.”

The term “antigen-binding moiety” refers to a polypeptide or a set of interacting polypeptides that specifically bind to an antigen, and includes, but is not limited to, an antibody (e.g., a monoclonal antibody, polyclonal antibody, a multi-specific antibody, a dual specific or bispecific antibody, an anti-idiotypic antibody, or a bifunctional hybrid antibody) or an antigen-binding fragment thereof (e.g., a Fab, a Fab′, a F(ab′)₂, a Fv, a disulfide linked Fv, a scFv, a single domain antibody (dAb), or a diabody), a single chain antibody, and an Fc-containing polypeptide such as an immunoadhesin. In some embodiments, the antibody may be of any heavy chain isotype (e.g., IgG, IgA, IgM, IgE, or IgD) or subtype (e.g., IgG₁, IgG₂, IgG₃, or IgG₄). In some embodiments, the antibody may be of any light chain isotype (e.g., kappa or lambda). The antibody may be human, non-human (e.g., from mouse, rat, rabbit, goat, or another non-human animal), chimeric (e.g., with a non-human variable region and a human constant region), or humanized (e.g., with non-human CDRs and human framework and constant regions). In some embodiments, the antibody is a derivatized antibody.

The term “cytokine agonist polypeptide” refers to a wildtype cytokine, or an analog thereof. An analog of a wildtype cytokine has the same biological specificity (e.g., binding to the same receptor(s) and activating the same target cells) as the wildtype cytokine, although the activity level of the analog may be different from that of the wildtype cytokine. The analog may be, for example, a mutein (i.e., mutated polypeptide) of the wildtype cytokine, and may comprise at least one, at least two, at least three, at least four, at least five, at least six, at least seven, at least eight, at least nine, or at least ten mutations relative to the wildtype cytokine.

The term “cytokine antagonist” or “cytokine mask” refers to a moiety (e.g., a polypeptide) that binds to a cytokine and thereby inhibiting the cytokine from binding to its receptor on the surface of a target cell and/or exerting its biological functions while being bound by the antagonist or mask. Examples of a cytokine antagonist or mask include, without limitations, a polypeptide derived from an extracellular domain of the cytokine's natural receptor that makes contact with the cytokine.

The term “effective amount” or “therapeutically effective amount” refers to an amount of a compound or composition sufficient to treat a specified disorder, condition, or disease, such as ameliorate, palliate, lessen, and/or delay one or more of its symptoms. In reference to a disease such as cancer, an effective amount may be an amount sufficient to delay cancer development or progression (e.g., decrease tumor growth rate, and/or delay or prevent tumor angiogenesis, metastasis, or infiltration of cancer cells into peripheral organs), reduce the number of epithelioid cells, cause cancer regression (e.g., shrink or eradicate a tumor), and/or prevent or delay cancer occurrence or recurrence. An effective amount can be administered in one or more administrations.

The term “functional analog” refers to a molecule that has the same biological specificity (e.g., binding to the same ligand) and/or activity (e.g., activating or inhibiting a target cell) as a reference molecule.

The term “fused” or “fusion” in reference to two polypeptide sequences refers to the joining of the two polypeptide sequences through a backbone peptide bond. Two polypeptides may be fused directly or through a peptide linker that is one or more amino acids long. A fusion polypeptide may be made by recombinant technology from a coding sequence containing the respective coding sequences for the two fusion partners, with or without a coding sequence for a peptide linker in between. In some embodiments, fusion encompasses chemical conjugation.

The term “pharmaceutically acceptable excipient” when used to refer to an ingredient in a composition means that the excipient is suitable for administration to a treatment subject, including a human subject, without undue deleterious side effects to the subject and without affecting the biological activity of the active pharmaceutical ingredient (API).

The term “subject” refers to a mammal and includes, but is not limited to, a human, a pet (e.g., a canine or a feline), a farm animal (e.g., cattle or horse), a rodent, or a primate.

As used herein, “treatment” or “treating” is an approach for obtaining beneficial or desired clinical results. Beneficial or desired clinical results include, but are not limited to, one or more of the following: alleviating one or more symptoms resulting from a disease, diminishing the extent of a disease, ameliorating a disease state, stabilizing a disease (e.g., preventing or delaying the worsening or progression of the disease), preventing or delaying the spread (e.g., metastasis) of a disease, preventing or delaying the recurrence of a disease, providing partial or total remission of a disease, decreasing the dose of one or more other medications required to treat a disease, increasing the patient's quality of life, and/or prolonging survival. The methods of the present disclosure contemplate any one or more of these aspects of treatment.

It is to be understood that one, some or all of the properties of the various embodiments described herein may be combined to form other embodiments of the present invention. The section headings used herein are for organizational purposes only and are not to be construed as limiting the subject matter described thereunder.

### IL-15 Prodrugs

The present disclosure IL-15 prodrugs that are metabolized in vivo to become active IL-15 therapeutics. The IL-15 prodrugs have fewer side effects, better in vivo PK profiles (e.g., longer half-life) and better target specificity, and are more efficacious as compared to prior IL-15 therapeutics. The IL-15 prodrugs of the present disclosure have configurations that lead to lower levels of aggregation and improved manufacturing efficiency, thereby overcoming common challenges in the manufacturing of fusion molecules and bispecific molecules.

The present prodrugs comprise an IL-15 polypeptide (A) (i.e., a cytokine agonist polypeptide or IL-15 cytokine moiety), an optional IL-15Rα Sushi domain (S), a masking moiety (M) (i.e., a cytokine antagonist) and a carrier moiety (C). The components are operationally linked to each other through peptide linkers, one of which may be cleavable such that upon activation by proteases at a target site, the masking moiety and the IL-15 cytokine moiety detach from each other. In some embodiments, the masking moiety (IL-15 antagonist), which may be, for example, an extracellular domain of a receptor for IL-15 or a binding fragment of an antibody which binds to the cytokine, is linked to the cytokine moiety, to the Sushi domain, or to the carrier moiety through a cleavable linker (e.g., a cleavable peptide linker). In other embodiments, the masking moiety is linked to the other moiety through a noncleavable linker.

The mask inhibits the IL-15 cytokine moiety's biological functions while the mask is binding to it. In some embodiments, a masking moiety of the present prodrugs specifically binds to an epitope located on the IL-2Rβ- and/or γ-chain interacting domain of the IL-15 polypeptide. A masking moiety's inhibitory effect may be removed upon protease digestion of the cleavable linker in the prodrug, allowing the masking moiety and the cytokine moiety to separate. In some embodiments, a masking moiety of the present prodrugs does not block or interfere with the binding of the IL-15 polypeptide (A) to IL-15Rα. The prodrugs may be activated at a target site (e.g., at a tumor site or the surrounding environment, or an infection site) in the patient by cleavage of the linker and the consequent release of the cytokine mask or the IL-15 cytokine moiety from the remainder of the prodrug, exposing the previously masked IL-15 cytokine moiety and allowing the IL-15 cytokine moiety to bind to its receptor on a target cell and exert its biological functions on the target cell. In some embodiments, the carriers for the prodrugs are antigen-binding moieties, such as antibodies, that bind an antigen at the target site.

In some embodiments of the IL-15 prodrugs of the present disclosure, the Sushi domain is fused to the carrier, the masking moiety, and/or the IL-15 cytokine moiety through a peptide linker (noncleavable or cleavable). In some embodiments, the IL-15 cytokine moiety is fused to the carrier moiety, the masking moiety, and/or the Sushi domain through a peptide linker (noncleavable or cleavable). In some embodiments, the masking moiety is fused to the carrier moiety, the cytokine moiety, and/or the Sushi domain through a peptide linker (noncleavable or cleavable).

In some embodiments, the present prodrugs are metabolized to become active IL-15 cytokines, which are pro-inflammatory, at a target site in the body targeted by the carrier. In further embodiments, the carrier in the prodrug is an antibody targeting a tumor antigen such that the prodrug is delivered to a tumor site in a patient and is metabolized locally (e.g., inside or in the vicinity of the tumor microenvironment) through cleavage of the linker linking the cytokine mask to the carrier or the cytokine moiety, making the pro-inflammatory cytokine moiety available to interact with its receptor on a target cell and stimulating the target immune cells locally.

### A. IL-15 Moieties of the Prodrugs

In the present IL-15 prodrugs, the IL-15 cytokine moiety may be a wildtype IL-15 polypeptide such as a wildtype human IL-15 polypeptide (SEQ ID NO: 2), or an IL-15 mutein, such as an IL-15 mutein derived from a human wildtype IL-15, with reduced affinity for IL-2Rβ (CD122) compared to wild type IL-15. The IL-15 mutein may have significantly reduced affinity for CD122 or the dimeric IL-2R, as compared to the wild type IL-15.

In some embodiments, the IL-15 moiety, when masked, has its biological activity reduced by at least 5 times, at least 10 times, at least 20 times, at least 50 times, or at least 100 times; or has its EC₅₀ value increased by at least 5 times, at least 10 times, at least 20 times, at least 50 times or at least 100 times.

In some embodiments, the IL-15 moiety is an IL-15 mutein comprising at least 1, 2, 3, 4, or 5 mutations at positions selected from N1, N4, 16, S7, D8, K10, K11, E46, D61, T62, E64, N65, I68, L69, N72, V63, L66, I67, A70, N71, Q108, N112 of human IL-15. Exemplary IL-15 muteins are those with one or more mutations selected from N1A, N1D, N4A, N4D, I6T, S7A, D8A, DAT, D8E, D8N, K10A, K10D, K11A, K11D, D61A, D61N, T62L, T62A, E64A, E64L, E64K, E64Q, N65A, N65L, N65D, L66D, L66E, I 67D, I67E, I68S, 168E, L69S, L69E, N72A, N72D, V63E, V63D, L66E, L66D, I67E, I67D, Q108E, and N112A. In some embodiments, the IL-15 moiety comprises a mutation or positions selected from E46, V49, L45, S51, and L52. Unless otherwise indicated, all residue numbers in IL-15 and IL-15 muteins described herein are in accordance with the numbering in SEQ ID NO: 2. In other embodiments, the IL-15 moiety comprises an amino acid sequence that is at least 90%, 91%, 92%, 93%, 94%, 95%, 96%, 97%, 98%, or 99% identical to SEQ ID NO: 2.

In particular embodiments, the IL-15 mutein contains mutations selected from N1D/D61N, N1D/E64Q, N4D/D61N, N4D/E64Q, D8N/D61N, D8N/E64Q, D3ON/E64Q/N65D, D61N/E64Q, E64Q/Q108E, N1D/N4D/D8N, D61N/E64Q/N65D, N1D/D61N/E64Q, N1D/D61N/E64Q/Q108E, and N4D/D61N/E64Q/Q108E.

### B. IL-15 Receptor Alpha Sushi Domain

In some embodiments, the present IL-15 prodrug comprises an IL-15Rα Sushi domain. The Sushi domain may be fused to the carrier directly or to the IL-15 cytokine moiety, optionally through a linker (e.g., a noncleavable or cleavable peptide linker). The masking moiety may be fused to the Sushi domain or to the carrier through a cleavable or noncleavable peptide linker. In a particular embodiment, the Sushi domain is fused to the carrier and the cytokine moiety is fused to the Sushi domain through a peptide linker. In the present IL-15 prodrugs, the Sushi domain may be a wild-type Sushi domain, or a Sushi domain comprising an amino acid sequence of SEQ ID NO: 7 or 9. In other embodiments, the Sushi domain comprises an amino acid sequence that is at least 90%, 91%, 92%, 93%, 94%, 95%, 96%, 97%, 98%, or 99% identical to SEQ ID NO: 7 or SEQ ID NO: 9.

In some embodiments, the human IL-15 receptor alpha (IL-15Rα) protein has the amino acid sequence set forth in SEQ ID NO: 8. In some cases, the coding sequence of human IL-15Rα is set forth in SEQ ID NO: 137. An exemplary IL-15Rα protein of the prodrug outlined herein can comprise or consist of the Sushi domain of SEQ ID NO: 8 (e.g., amino acids 31-95 or 31- 105 of SEQ ID NO: 8), or in other words, the amino acid sequence of SEQ ID NO: 9 or SEQ ID NO: 7. In some embodiments, the IL-15Rα protein has the amino acid sequence of SEQ ID NO: 7 and an amino acid insertion selected from the group consisting of D96, P97, A98, D96/P97, D96/C97, D96/P97/A98, D96/P97/C98, and D96/C97/A98, wherein the amino acid position is relative to full-length human IL-15Rα protein or SEQ ID NO: 8. For instance, amino acid(s) such as D, P, A, DP, DC, DPA, DPC, or DCA can be added to the C-terminus of the IL-15Rα protein (e.g., SEQ ID NO: 9). In some embodiments, the IL-15Rα protein has the amino acid sequence of SEQ ID NO: 9 and one or more amino acid substitutions selected from the group consisting of K34C, A37C, G38C, 540C, and L42C, wherein the amino acid position is relative to SEQ ID NO:9. In certain embodiments, the IL-15 analog and the Sushi domain have a set of amino acid substitutions or additions selected from the group consisting of E87C: D96/P97/C98; E87C:D96/C97/A98; V49C: 540C; L52C: 540C; E89C: K34C; Q48C: G38C; E53C: L42C; C42S: A37C; and L45C: A37C, respectively (the mutations in IL-15 are shown before the colon; and the mutations in the Sushi domain are shown after the colon).

### C. Masking Moieties of the Prodrugs

The cytokine antagonist, i.e., the masking moiety, in the present prodrug may comprise a peptide or an antibody or antibody fragment that binds to the cytokine moiety in the prodrug, masking the cytokine moiety and inhibiting its biological functions. In some embodiments, the masking moiety comprises an antigen-binding moiety or a binding fragment of an antibody, which binds to a human IL-15 polypeptide and inhibits a biological activity of the IL-15 polypeptide.

By way of example, IL-15 antagonists may comprise peptides and antibodies that bind IL-15 and interfere with the binding of the IL-15 moiety to its receptors, leading to the reduced biological activities of the IL-15 moiety while masked. In some embodiments, the IL-15 antagonist comprises an IL-2Rβ or IL-2Rγ extracellular domain or its functional analog such as one derived from human IL-2Rβ or IL-2Rγ (e.g., one of SEQ ID NOs: 3-6). In some embodiments, the IL-15 antagonist comprises a peptide identified from the screening of a peptide library. In some embodiments, the IL-15 antagonist comprises an antibody or fragment thereof that blocks the binding of IL-15 or IL-15 muteins to an IL-15 receptor. In other embodiments, the antagonist inhibits biological activity of an IL-15 polypeptide. In some embodiments, the antagonist comprises a scFv, a Fab, or other type of antibody fragment known in the art. In preferred embodiments, the antibody fragment is a scFv specific for IL-15. In other preferred embodiments, the antagonist specifically binds to an epitope located on the β- and/or γ-chain interacting domain of the IL-15 agonist polypeptide. In particular embodiments, the masking moiety does not block or interfere with the binding of the IL-15 polypeptide to IL-15Rα. By way of example, the IL-15-binding antibody may be selected from 146B7, 146H5, 404E4, and 404A8. In some embodiments, a scFv or Fab IL-15 antagonist comprises the CDR1, CDR2 and CDR3 domains of an anti-IL-15 antibody selected from 146B7, 146H5, 404E4, and 404A8; and the CDR1, CDR2 and CDR3 domains from the light chain of an anti-IL-15 antibody selected from 146B7, 146H5, 404E4, and 404A8, all of which are described in described in WO2003/017935A2.

In some embodiments, an IL-15 antagonist comprises heavy chain CDR1, CDR2 and CDR3 domains with amino acid sequences of SEQ ID NO: 100, 101, and 102, respectively; and light chain CDR1, CDR2 and CDR3 domains with amino acid sequences of SEQ ID NO: 103, 104, and 105, respectively. In some embodiments, the heavy chain CDR3 domain of SEQ ID NO: 102 comprises a substitution mutation of its Cys residue. The Cys residue within the CDR3 domain of SEQ ID NO: 102 may be mutated to Ser, Thr, Ala, Asn, or Gln. In another embodiment, the CDR3 domain comprises the amino acid sequence of SEQ ID NO: 106. In some embodiments, the antagonist or masking moiety is a scFv or a Fab comprising a heavy chain variable domain with an amino acid sequence of SEQ ID NO: 107 or at least 95% identical to SEQ ID NO: 107, and a light chain variable domain with an amino acid sequence of SEQ ID NO: 108 or 123 or at least 95% identical to SEQ ID NO: 108 or 123. In some specific moiety, the masking moiety comprises an amino acid sequence SEQ ID NO: 110 or 124.

### D. Carrier Moieties of the Prodrugs

The carrier moieties of the present prodrugs may be an antigen-binding moiety, or a moiety that is not an antigen-binding moiety. The carrier moiety may improve the PK profiles such as serum half-life of the cytokine agonist polypeptide, and may also target the cytokine agonist polypeptide to a target site in the body, such as a tumor site.

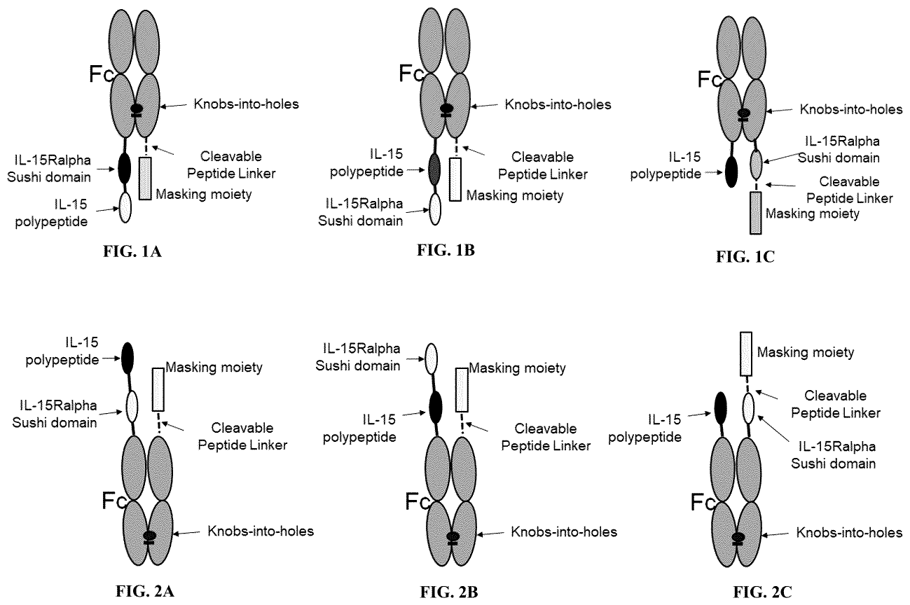

In some embodiments, the carrier moiety (C) is an Fc domain comprising a first and a second polypeptide chain (i.e., two different heavy chains), wherein said polypeptide chains comprise molecular formulas (from N-terminus to C-terminus) selected from one of the following pairs:
a) F1-PL1-A-PL2-S, F2-CL-M (FIG. 1A);
b) F1-PL1-S-PL2-A, F2-CL-M (FIG. 1B); and
c) F1-PL1-S-PL2-A, F2-CL-M (FIG. 1C);
   wherein F1 and F2 are subunits of the carrier moiety (e.g., Fc domain), which form a heterodimer; PL1 and PL2 are peptide linkers; CL is a cleavable peptide linker; S is the Sushi domain; and A is an IL-15 polypeptide.

In some embodiments, the carrier moiety (C) is an Fc domain comprising a first and a second polypeptide chain (i.e., two different heavy chains), wherein said polypeptide chains comprise molecular formulas (from N-terminus to C-terminus) selected from one of the following pairs:
a) A-PL1-S-F1, M-CL-F2 (FIG. 2A);
b) S-PL1-A-F1, M-CL-F2 (FIG. 2B); and
c) A-PL1-F1, M-CL-S-F2 (FIG. 2C);
   wherein F1 and F2 are subunits of the carrier moiety (e.g., Fc domain), which form a heterodimer; PL1 and PL2 are peptide linkers; CL is a cleavable peptide linker; S is the Sushi domain; and A is an IL-15 polypeptide.

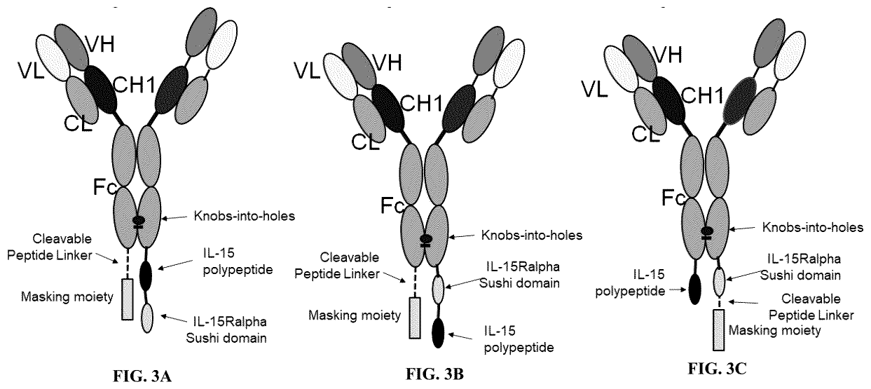

In some embodiment, the carrier moiety (C) is an antibody comprising two light chains of an antibody, a first antibody heavy chain, and a second antibody heavy chain, wherein
a) the first heavy chain comprises the molecular formula (from N-terminal to C-terminal) C1-CL-M; and
b) the second heavy chain comprises the molecular formula (from N-terminal to C-terminal) C2-PL1-S-PL2-A,
wherein the C1 and C2 are the antibody heavy chains; said PL1 and PL2 are peptide linkers; CL is a cleavable peptide linker; S is the Sushi domain; and A is an IL- 15 polypeptide. In other embodiments, the order of the above first and second heavy chains are reversed (FIGS. 3A and 3B).

In some embodiment, the carrier moiety (C) is an antibody comprising two light chains of an antibody, a first antibody heavy chain, and a second antibody heavy chain, wherein
a) the first heavy chain comprises the molecular formula (from N-terminal to C-terminal) C1-A; and
b) the second heavy chain polypeptide chain comprises the molecular formula (from N-terminal to C-terminal) C2-PL1-S-CL-M,
wherein the C1 and C2 are the antibody heavy chains; said PL1 and PL2 are peptide linkers; CL is a cleavable peptide linker; S is the Sushi domain; and A is an IL-15 polypeptide (FIG. 3C).

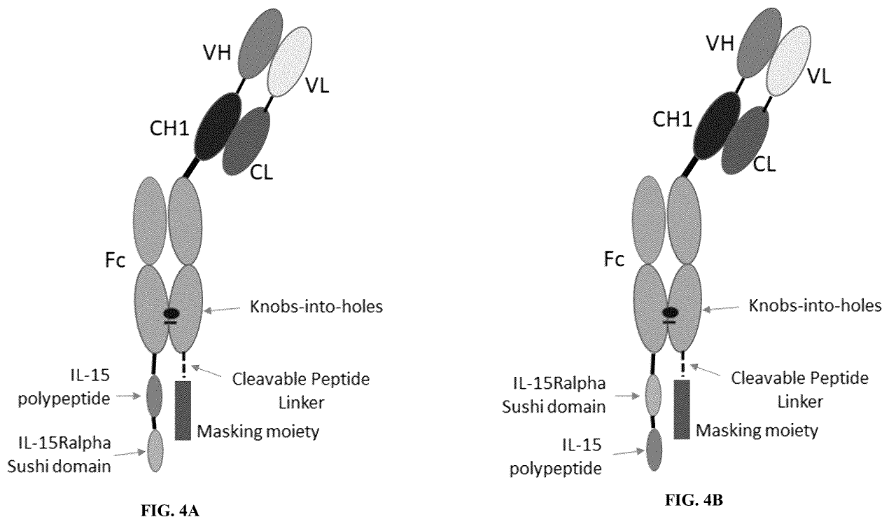

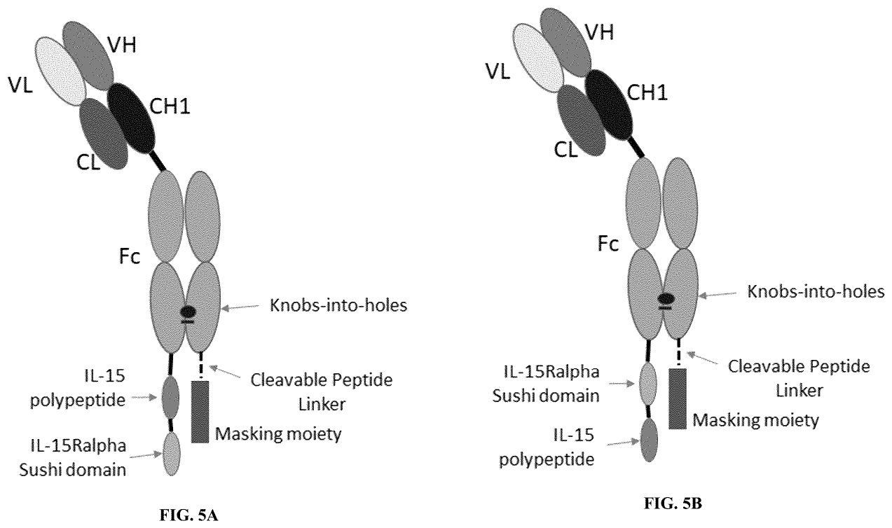

In some embodiments, the prodrugs of the present disclosure comprise three polypeptide chains—one antibody light chain and two heavy chains, —wherein the first polypeptide chain is an antibody light chain variable region, the first heavy chain comprises an antibody's heavy chain variable and constant regions, and the second heavy chain comprises a CH2 and a CH3 domain, wherein the first and second heavy chains comprise molecular formulas (from N-terminal to C-terminal) selected from one of the following pairs:
a) F-PL1-A-PL2-S, HC-CL-M (FIG. 4A);
b) F-PL1-S-PL2-A, HC-CL-M (FIG. 4B);
c) HC-PL1-A-PL2-S, F-CL-M (FIG. 5A); and
d) HC-PL1-S-PL2-A, F-CL-M (FIG. 5B).
wherein F is a subunit of a Fc domain (comprising the CH2 and CH3 domains); HC is the heavy chain of an antibody which forms an antigen binding moiety with said light chain; PL1 and PL2 are peptide linkers; CL is a cleavable peptide linker; S is the Sushi domain; and A is an IL-15 polypeptide.

### 1. Antigen-Binding Carrier Moieties

The carrier moiety may be an antibody or an antigen-binding fragment thereof, or an immunoadhesin. In some embodiments, the antigen-binding moiety is a full-length antibody with two heavy chains and two light chains, a Fab fragment, a Fab′ fragment, a F(ab′)₂ fragment, a Fv fragment, a disulfide linked Fv fragment, a single domain antibody, a nanobody, or a single-chain variable fragment (scFv). In some embodiments, the antigen-binding moiety is a bispecific antigen-binding moiety and can bind to two different antigens or two different epitopes on the same antigen. The antigen-binding moiety may provide additional and potentially synergetic therapeutic efficacy to the cytokine agonist polypeptide.

The cytokine (IL-15) polypeptide and its mask may be fused to the N-terminus or C-terminus of the light chains and/or heavy chains of the antigen-binding moiety. By way of example, the cytokine (e.g., IL-15 polypeptide and its mask may be fused to the antibody heavy chain or an antigen-binding fragment thereof or to the antibody light chain or an antigen-binding fragment thereof. In some embodiments, the cytokine (IL-15) polypeptide is fused to the C-terminus of one or both of the heavy chains of an antibody, and the cytokine's mask is fused to the other terminus of the heavy chain, or to the C-terminus of the cytokine agonist polypeptide, through a cleavable or noncleavable peptide linker. In some embodiments, the cytokine (IL-15) polypeptide is fused to the C-terminus of one of the heavy chains of an antibody, and the cytokine's mask is fused to the C-terminus of the other heavy chain of the antibody through a cleavable peptide linker, wherein the two heavy chains optionally contain mutations that allow the specific pairing of the two different heavy chains.

Strategies of forming heterodimers for Fc-fusion polypeptides or bispecific antibodies are well known (see, e.g., Spies et al., Mol Imm. (2015) 67(2)(A):95-106). For example, the two heavy chain polypeptides in the prodrug may form stable heterodimers through “knobs-into-holes” mutations. “Knobs-into-holes” mutations are made to promote the formation of the heterodimers of the antibody heavy chains and are commonly used to make bispecific antibodies (see, e.g., U.S. Pat. No. 8,642,745). For example, the Fc domain of the antibody may comprise a T366W mutation in the CH3 domain of the “knob chain” and T366S, L368A, and/or Y407V mutations in the CH3 domain of the “hole chain.” An additional interchain disulfide bridge between the CH3 domains can also be used, e.g., by introducing a Y349C mutation into the CH3 domain of the “knobs chain” and an E356C or S354C mutation into the CH3 domain of the “hole chain” (see, e.g., Merchant et al., Nature Biotech (1998)16:677-81). In other embodiments, the antibody moiety may comprise Y349C and/or T366W mutations in one of the two CH3 domains, and E356C, T366S, L368A, and/or Y407V mutations in the other CH3 domain. In certain embodiments, the antibody moiety may comprise Y349C and/or T366W mutations in one of the two CH3 domains, and S354C (or E356C), T366S, L368A, and/or Y407V mutations in the other CH3 domain, with the additional Y349C mutation in one CH3 domain and the additional E356C or S354C mutation in the other CH3 domain, forming an interchain disulfide bridge (numbering always according to EU index of Kabat; Kabat et al., “Sequences of Proteins of Immunological Interest,” 5th ed., Public Health Service, National Institutes of Health, Bethesda, Md. (1991)). Other knobs-into-holes technologies, such as those described in EP1870459A1, can be used alternatively or additionally. Thus, another example of knobs-into-holes mutations for an antibody moiety is having R409D/K370E mutations in the CH3 domain of the “knob chain” and D399K/E357K mutations in the CH3 domain of the “hole chain” (EU numbering).

In some embodiments, the antibody moiety in the prodrug comprises L234A and L235A (“LALA”) mutations in its Fc domain. The LALA mutations eliminate complement binding and fixation as well as Fcγ dependent ADCC (see, e.g., Hezareh et al. J. Virol. (2001) 75(24):12161-8). In further embodiments, the LALA mutations are present in the antibody moiety in addition to the knobs-into-holes mutations.

In some embodiments, the antibody moiety comprises the M252Y/S254T/T256E (“YTE”) mutations in the Fc domain. The YTE mutations allow the simultaneous modulation of serum half-life, tissue distribution and activity of IgG₁ (see Dall'Acqua et al., J Biol Chem. (2006) 281: 23514-24; and Robbie et al., Antimicrob Agents Chemother. (2013) 57(12):6147-53). In further embodiments, the YTE mutations are present in the antibody moiety in addition to the knobs-into-holes mutations. In particular embodiments, the antibody moiety has YTE, LALA and knobs-into-holes mutations or any combination thereof.

The antigen-binding moiety may bind to an antigen on the surface of a cell, such as an immune cell, for example, T cells, NK cells, and macrophages, or bind to a cytokine. For example, the antigen-binding moiety may bind to PD-1, LAG-3, TIM-3, TIGIT, CTLA-4, or TGF-beta and may be an antibody. The antibody may have the ability to activate the immune cell and enhance its anti-cancer activity.

The antigen-binding moiety may bind to an antigen on the surface of a tumor cell. For example, the antigen-binding moiety may bind to FAP alpha, 5T4, Trop-2, PD-L1, HER-2, EGFR, Claudin 18.2, DLL-3, GCP3, or carcinoembryonic antigen (CEA), and may be an antibody. The antibody may or may not have ADCC activity. The antibody may also be further conjugated to a cytotoxic drug.

In some embodiments, the antigen-binding moiety binds to guanyl cyclase C (GCC), carbohydrate antigen 19-9 (CA19-9), glycoprotein A33 (gpA33), mucin 1 (MUC1), insulin-like growth factor 1 receptor (IGF1-R), human epidermal growth factor receptor 2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGFR1), vascular endothelial growth factor receptor 2 (VEGFR2), Nectin-4, Liv-1, glycoprotein NMB (GPNMB), prostate-specific membrane antigen (PSMA), Trop-2, carbonic anhydrase IX (CA9), endothelin B receptor (ETBR), six transmembrane epithelial antigen of the prostate 1 (STEAP1), folate receptor alpha (FRα), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD33, CD40, CD56, CD66e, CD70, CD74, CD79b, CD98, CD123, CD138, CD352, CD47, signal-regulatory protein alpha (SIRPα), Claudin 18.2, Claudin 6, BCMA, or EPCAM. In some embodiments, the antigen-binding moiety binds to an epidermal growth factor (EGF)-like domain of DLL3. In some embodiments, the antigen-binding moiety binds to a Delta/Serrate/Lag2 (DSL)-like domain of DLL3. In some embodiments, the antigen-binding moiety binds to an epitope located after the 374th amino acid of GPC3. In some embodiments, the antigen-binding moiety binds to a heparin sulfate glycan of GPC3. In some embodiments, the antigen-binding moiety binds to Claudin 18.2 and does not bind to Claudin 18.1. In some embodiments, the antigen-binding moiety binds to Claudin 18.1 with at least 10 times weaker binding affinity than to Claudin 18.2.

In some embodiments, the antigen-binding moiety (carrier moiety) includes an antibody or fragment thereof known in the art that binds to PD-1 and disrupts the interaction between the PD-1 and its ligand (PD-L1) to stimulate an anti-tumor immune response. In some embodiments, the antibody or antigen-binding portion thereof binds specifically to PD-1. For example, antibodies that target PD-1 and which can find use in the present invention include, but are not limited to, nivolumab (BMS-936558, Bristol-Myers Squibb), pembrolizumab (lambrolizumab, MK03475 or MK-3475, Merck), humanized anti-PD-1 antibody JS001 (ShangHai JunShi), monoclonal anti-PD-1 antibody TSR-042 (Tesaro, Inc.), pidilizumab (anti-PD-1 mAb CT-011, Medivation), anti-PD-1 monoclonal Antibody BGB-A317 (BeiGene), and/or anti-PD-1 antibody SHR-1210 (ShangHai HengRui), human monoclonal antibody REGN2810 (Regeneron), human monoclonal antibody MDX-1106 (Bristol-Myers Squibb), and/or humanized anti-PD-1 IgG4 antibody PDR001 (Novartis). In some embodiments, the PD-1 antibody is from clone: RMP1-14 (rat IgG)—BioXcell cat# BP0146. Other suitable anti-PD-1 antibodies include those disclosed in U.S. Pat. No. 8,008,449. In some embodiments, the antibody or antigen-binding portion thereof binds specifically to PD-L1 and inhibits its interaction with PD-1, thereby increasing immune activity. Any antibodies known in the art which bind to PD-L1 and disrupt the interaction between the PD-1 and PD-L1, and stimulates an anti-tumor immune response, are suitable for use in combination treatment methods disclosed herein. As an example, antibodies that target PD-Ll include BMS-936559 (Bristol-Myers Squibb) and MPDL3280A (Genetech; currently in human trials). Other suitable antibodies that target PD-L1 are disclosed in U.S. Pat. No. 7,943,743. It will be understood by one of ordinary skill that any antibody which binds to PD-1 or PD-L1, disrupts the PD-1/PD-L1 interaction, and stimulates an anti-tumor immune response, is suitable for use in the combination treatment methods disclosed herein.

In some embodiments, wherein the carrier is an antibody against human PD-L1, which is selected from ASKB1296, avelumab, atezolizumab and durvalumab.

In some embodiments, the carrier is an antibody, which binds to an antigen expressed on a cancer cell. In some embodiments, the carrier antibody has ADCC activity. In some embodiments, the carrier antibody binds to an antigen selected from HER2, HER3, EGFR, CMET, Trop-2, GPC3, Claudin 18.2, Claudin 6, 5T4, BCMA, CD38, CD20, CD30, CD47, and VEGFR2.

In some embodiments, the carrier is a bispecific antibody which binds to two antigens selected from PD-1, PD-L1, CTLA-4, LAG-4, TIM-3, CD47, and TIGIT.

In some embodiments, the carrier antibody binds to human PD-1, wherein the PD-1 antibody comprises the same heavy chain CDR1, CDR2 and CDR3 domains, and light chain CDR1, CDR2, and CDR3 domains as derived from the heavy chain and light chain of nivolumab, pembrolizumab, toripalimab, sintilimab, or tislelizumab.

In some embodiments, the carrier antibody binds to human PD-1, wherein the light chain comprises an amino acid sequence at least 99% identical as one selected from SEQ ID NO: 55 and 56; wherein the first heavy chain polypeptide chain comprises an amino acid sequence at least 99% identical as that of SEQ ID NO: 54, 60, or 61; and wherein the second heavy chain polypeptide chain comprises an amino acid sequence at least 99% identical as one selected from SEQ ID NO: 52, 53, 58, 59, 62, 63 and 69.

In some embodiments, the antibody binds to human PD-1, wherein the light chain comprises an amino acid sequence at least 99% identical as SEQ ID NO: 55; wherein the first heavy chain polypeptide chain comprises an amino acid sequence at least 99% identical as that of SEQ ID NO: 66; and wherein the second heavy chain polypeptide chain comprises an amino acid sequence at least 99% identical as one selected from SEQ ID NO: 64, 65, 67 and 68.

In some embodiments, the carrier antibody binds to PD-1, wherein the light chain comprises an amino acid sequence at least 99% identical as one selected from SEQ ID NOs: 55 and 56; wherein the first heavy chain comprises an amino acid sequence at least 99% identical as one selected from SEQ ID NO: 80, 81, or 87; and wherein the second heavy chain comprises an amino acid sequence at least 99% identical as one selected from SEQ ID NOs: 52, 53, 58, 59, 62, 63 and 69.

In some embodiments, the carrier antibody binds to PD-1, wherein the light chain comprises an amino acid sequence at least 99% identical as one selected from SEQ ID NOs: 55 and 56; wherein the first heavy chain comprises an amino acid sequence at least 99% identical as that of SEQ ID NO: 54, 60, or 61; and wherein second heavy chain comprises an amino acid sequence at least 99% identical as one selected from SEQ ID NOs: 82, 83, 84, 85 and 86.

In some embodiments, the carrier antibody binds to PD-L1, wherein the light chain comprises an amino acid sequence at least 99% identical as that of SEQ ID NO: 50 or 51; wherein the first heavy chain polypeptide chain comprises an amino acid at least 99% identical as that of SEQ ID NO: 47, 48 or 49; and wherein the second heavy chain polypeptide chain comprises an amino acid sequence at least 99% identical as that of SEQ ID NO: 45 or 46.

In some embodiments, the carrier antibody is a bispecific antibody, which binds to two antigens selected from HER2, HER3, EGFR, CMET, Trop-2, GPC3, Claudin 18.2, Claudin 6, 5T4, BCMA, CD38, CD20, CD30, and VEGFR2. In some embodiments, the carrier is a bispecific antibody, which binds to cMet and EGFR; wherein the EGFR binding domain comprises light chain CDR1, CDR2 and CDR3 derived from SEQ ID NO: 88 or 90, and heavy chain CDR1, CDR2, and CDR3 derived from SEQ ID NO: 89 or 91.

In some embodiments, the carrier moiety is an IgG1 Fc domain; and wherein the first polypeptide comprises an amino acid sequence at least 99% identical as one selected from SEQ ID NO: 37, 70-72 and 73, and the second polypeptide chain comprises an amino acid sequence at least 99% identical as one selected from SEQ ID NOs: 38, 39, 75-78, and 79.

In some embodiments, the carrier moiety is an IgG4 Fc domain; and wherein the first polypeptide comprises an amino acid sequence at least 99% identical as one shown in SEQ ID NO: 80, 81 or 87, and the second polypeptide chain comprises an amino acid sequence at least 99% identical as one selected from SEQ ID NOs: 82-85 and 86.

In some embodiments, the antigen-binding moiety includes an antibody or fragment thereof known in the art that binds CTLA-4 and disrupts its interaction with CD80 and CD86. Exemplary antibodies that target CTLA-4 include ipilimumab (MDX-010, MDX-101, Bristol-Myers Squibb), which is FDA approved, and tremelimumab (ticilimumab, CP-675, 206, Pfizer), which is currently undergoing human trials. Other suitable antibodies that target CTLA-4 are disclosed in WO 2012/120125, U.S. Pat. Nos. 6,984,720, 6,682,7368, and U.S. Patent Applications 2002/0039581, 2002/0086014, and 2005/0201994. It will be understood by one of ordinary skill that any antibody which binds to CTLA-4, disrupts its interaction with CD80 and CD86, and stimulates an anti-tumor immune response, is suitable for use in the combination treatment methods disclosed herein.

In some embodiments, the combination therapy includes an antibody known in the art that binds LAG-3 and disrupts its interaction with MEC class II molecules. An exemplary antibody that targets LAG-3 is IMP321 (Immutep), currently undergoing human trials. Other suitable antibodies that target LAG-3 are disclosed in U.S. Patent Application 2011/0150892. It will be understood by one of ordinary skill that any antibody which binds to LAG-3, disrupts its interaction with MEC class II molecules, and stimulates an anti-tumor immune response, is suitable for use in the combination treatment methods disclosed herein.

In some embodiments, the antigen-binding moiety comprises an antibody or fragment thereof known in the art that binds TIM-3 and disrupts its interaction with galectin 9. Suitable antibodies that target TIM-3 are disclosed in U.S. Patent Application 2013/0022623. It will be understood by one of ordinary skill that any antibody which binds to TIM-3, disrupts its interaction with galectin 9, and stimulates an anti-tumor immune response, is suitable for use in the combination treatment methods disclosed herein.

In some embodiments, the antigen-binding moiety comprises an antibody or fragment thereof known in the art that binds 4-1BB/CD137 and disrupts its interaction with CD137L. It will be understood by one of ordinary skill that any antibody which binds to 4-1BB/CD137, disrupts its interaction with CD137L or another ligand, and stimulates an anti-tumor immune response or an immune stimulatory response that results in anti-tumor activity overall, is suitable for use in the combination treatment methods disclosed herein.

In some embodiments, the antigen-binding moiety comprises an antibody or fragment thereof known in the art that binds GITR and disrupts its interaction with its ligand. It will be understood by one of ordinary skill that any antibody which binds to GITR, disrupts its interaction with GITRL or another ligand, and stimulates an anti-tumor immune response or an immune stimulatory response that results in anti-tumor activity overall, is suitable for use in the combination treatment methods disclosed herein.

In some embodiments, the antigen-binding moiety comprises an antibody or fragment thereof known in the art that binds OX40 and disrupts its interaction with its ligand. It will be understood by one of ordinary skill that any antibody which binds to OX40, disrupts its interaction with OX40L or another ligand, and stimulates an anti-tumor immune response or an immune stimulatory response that results in anti-tumor activity overall, is suitable for use in the combination treatment methods disclosed herein.

In some embodiments, the antigen-binding moiety comprises an antibody or fragment thereof known in the art that binds CD40 and disrupts its interaction with its ligand. It will be understood by one of ordinary skill that any antibody which binds to CD40, disrupts its interaction with its ligand, and stimulates an anti-tumor immune response or an immune stimulatory response that results in anti-tumor activity overall, is suitable for use in the combination treatment methods disclosed herein.

In some embodiments, the antigen-binding moiety comprises an antibody or fragment thereof known in the art that binds ICOS and disrupts its interaction with its ligand. It will be understood by one of ordinary skill that any antibody which binds to ICOS, disrupts its interaction with its ligand, and stimulates an anti-tumor immune response or an immune stimulatory response that results in anti-tumor activity overall, is suitable for use in the combination treatment methods disclosed herein.

In some embodiments, the antigen-binding moiety comprises an antibody or fragment thereof known in the art that binds CD28 and disrupts its interaction with its ligand. It will be understood by one of ordinary skill that any antibody which binds to CD28, disrupts its interaction with its ligand, and stimulates an anti-tumor immune response or an immune stimulatory response that results in anti-tumor activity overall, is suitable for use in the combination treatment methods disclosed herein.

Additional exemplary antigen-binding moieties (carrier moieties) include trastuzumab, rituximab, brentuximab, cetuximab, panitumumab, GC33 (or a humanized version thereof), and anti-EGFR antibody mAb806 (or a humanized version thereof). In some embodiments, the antigen-binding moiety has at least 90%, 91%, 92%, 93%, 94%, 95%, 96%, 97%, 98%, or 99% identity to trastuzumab, rituximab, brentuximab, cetuximab, or panitumumab, GC33 (or a humanized version thereof), or anti-EGFR antibody mAb806 (or a humanized version thereof). In some embodiments, the antigen-binding moiety has an antibody heavy chain with at least 90%, 91%, 92%, 93%, 94%, 95%, 96%, 97%, 98%, or 99% identity to the antibody heavy chain of trastuzumab, rituximab, brentuximab, cetuximab, panitumumab, GC33 (or a humanized version thereof), anti-EGFR antibody mAb806 (or a humanized version thereof), or a fragment thereof. In some embodiments, the antigen-binding moiety has an antibody light chain with at least 90%, 91%, 92%, 93%, 94%, 95%, 96%, 97%, 98%, or 99% identity to the antibody light chain of trastuzumab, rituximab, brentuximab, cetuximab, panitumumab, GC33 (or a humanized version thereof), anti-EGFR antibody mAb806 (or a humanized version thereof), or a fragment thereof. The antigen-binding moiety is fused to an IL-15 polypeptide. In some embodiments, the antigen-binding moiety comprises the six complementarity-determining regions (CDRs) of trastuzumab, rituximab, brentuximab, cetuximab, panitumumab, GC33, or anti-EGFR antibody mAb806.

A number of CDR delineations are known in the art and are encompassed herein. A person of skill in the art can readily determine a CDR for a given delineation based on the sequence of the heavy or light chain variable region. The “Kabat” CDRs are based on sequence variability and are the most commonly used (Kabat et al., Sequences of Proteins of Immunological Interest, 5th Ed. Public Health Service, National Institutes of Health, Bethesda, Md. (1991)). “Chothia” CDRs refer to the location of the structural loops (Chothia & Lesk, Canonical structures for the hypervariable regions of immunoglobulins, J. Mol. Biol., vol. 196, pp. 901-917 (1987)). The “AbM” CDRs represent a compromise between the Kabat CDRs and Chothia structural loops are used by Oxford Molecular's AbM antibody modeling software. The “Contact” CDRs are based on an analysis of the available complex crystal structures. The residues from each of these CDRs are noted below in Table 1, in reference to common antibody numbering schemes. Unless otherwise specified herein, amino acid numbers in antibodies refer to the Kabat numbering scheme as described in Kabat et al., supra, including when CDR delineations are made in reference to Kabat, Chothia, AbM, or Contact schemes. Using this numbering system, the actual linear amino acid sequence may contain fewer or additional amino acids corresponding to a shortening of, or insertion into, a framework region (FR) or CDR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

TABLE 1
CDR Delineations According to Various Schemes

| CDR | Kabat | AbM | Chothia | Contact |
|---|---|---|---|---|
| VL-CDR1 | L24-L34 | L24-L34 | L26-L32 | L30-L36 |
| VL-CDR2 | L50-L56 | L50-L56 | L50-L52 | L46-L55 |
| VL-CDR3 | L89-L97 | L89-L97 | L91-L96 | L89-L96 |
| VH-CDR1 | H31-H35B | H26-H35B | H26-H32 | H30-H35B |
| (Kabat nos.) | | | | |
| VH-CDR1 | H31-H35 | H26-H35 | H26-H32 | H30-H35 |
| (Chothia nos.) | | | | |
| VH-CDR2 | H50-H65 | H50-H58 | H53-H55 | H47-H58 |
| VH-CDR3 | H95-H102 | H95-H102 | H95-H101 | H93-H101 |

In some embodiments, the CDRs are “extended CDRs,” and encompass a region that begins or terminates according to a different scheme. For example, an extended CDR can be as follows: L24-L36, L26-L34, or L26-L36 (VL-CDR1); L46-L52, L46-L56, or L50-L55 (VL-CDR2); L91-L97 (VL-CDR3); H47-H55, H47-H65, H50-H55, H53-H58, or H53-H65 (VH-CDR2); and/or H93-H102 (VH-CDR3).

In some embodiments, the antigen-binding moiety binds to EGFR, and comprises a light chain having an amino acid sequence with at least 90%, 91%, 92%, 93%, 94%, 95%, 96%, 97%, 98%, or 99% identity to SEQ ID NO: 88, or a fragment thereof, and a heavy chain having an amino acid sequence with at least 90%, 91%, 92%, 93%, 94%, 95%, 96%, 97%, 98%, or 99% identity to SEQ ID NO: 89, or a fragment thereof. In some embodiments, the antigen-binding domain comprises CDR1, CDR2, and CDR3 from SEQ ID NO: 88, and CDR1, CDR2, and CDR3 from SEQ ID NO: 89.

In some embodiments, the antigen-binding moiety binds to EGFR, and comprises a light chain having an amino acid sequence with at least 90%, 91%, 92%, 93%, 94%, 95%, 96%, 97%, 98%, or 99% identity to SEQ ID NO: 90, or a fragment thereof, and a heavy chain having an amino acid sequence with at least 90%, 91%, 92%, 93%, 94%, 95%, 96%, 97%, 98%, or 99% identity to SEQ ID NO: 91, or a fragment thereof. In some embodiments, the antigen-binding domain comprises CDR1, CDR2, and CDR3 from SEQ ID NO: 90, and CDR1, CDR2, and CDR3 from SEQ ID NO: 91.

In some embodiments, the antigen-binding moiety binds to c-MET, and comprises a light chain having an amino acid sequence with at least 90%, 91%, 92%, 93%, 94%, 95%, 96%, 97%, 98%, or 99% identity to SEQ ID NO: 92, or a fragment thereof, and a heavy chain having an amino acid sequence with at least 90%, 91%, 92%, 93%, 94%, 95%, 96%, 97%, 98%, or 99% identity to SEQ ID NO: 93, or a fragment thereof. In some embodiments, the antigen-binding domain comprises CDR1, CDR2, and CDR3 from SEQ ID NO: 92, and CDR1, CDR2, and CDR3 from SEQ ID NO: 93.

In some embodiments, the antigen-binding moiety binds to GPC3, and comprises a light chain having an amino acid sequence with at least 90%, 91%, 92%, 93%, 94%, 95%, 96%, 97%, 98%, or 99% identity to SEQ ID NO: 94, or a fragment thereof, and a heavy chain having an amino acid sequence with at least 90%, 91%, 92%, 93%, 94%, 95%, 96%, 97%, 98%, or 99% identity to SEQ ID NO: 95, or a fragment thereof. In some embodiments, the antigen-binding domain comprises CDR1, CDR2, and CDR3 from SEQ ID NO: 94, and CDR1, CDR2, and CDR3 from SEQ ID NO: 95.

In some embodiments, the antigen-binding moiety binds to 5T4, and comprises a light chain variable domain having an amino acid sequence with at least 90%, 91%, 92%, 93%, 94%, 95%, 96%, 97%, 98%, or 99% identity to SEQ ID NO: 98 or 99, and a heavy chain variable domain having an amino acid sequence with at least 90%, 91%, 92%, 93%, 94%, 95%, 96%, 97%, 98%, or 99% identity to SEQ ID NO: 96 or 97, or a fragment thereof. In some embodiments, the antigen-binding domain comprises CDR1, CDR2, and CDR3 from SEQ ID NO: 98 or 99, and CDR1, CDR2, and CDR3 from SEQ ID NO: 96 or 97.

In some embodiments, the antigen-binding moiety binds to Trop-2, and comprises a light chain variable region comprising a CDR1 comprising an amino acid sequence of KASQDVSIAVA (SEQ ID NO:125), a CDR2 comprising an amino acid sequence of SASYRYT (SEQ ID NO:126), and a CDR3 comprising an amino acid sequence of QQHYITPLT (SEQ ID NO:127); and a heavy chain variable region comprising a CDR1 comprising an amino acid sequence of NYGMN (SEQ ID NO:128), a CDR2 comprising an amino acid sequence of WINTYTGEPTYTDDFKG (SEQ ID NO: 129), and a CDR3 comprising an amino acid sequence of GGFGSSYWYFDV(SEQ ID NO: 130).

In some embodiments, the antigen-binding moiety binds to mesothelin, and comprises light chain variable region comprising a CDR1 comprising an amino acid sequence of SASSSVSYMH (SEQ ID NO: 131), a CDR2 comprising an amino acid sequence of DTSKLAS (SEQ ID NO: 132), and a CDR3 comprising an amino acid sequence of QQWSGYPLT (SEQ ID NO: 133); and a heavy chain variable region comprising a CDR1 comprising an amino acid sequence of GYTMN (SEQ ID NO: 134), a CDR2 comprising an amino acid sequence of LITPYNGASSYNQKFRG (SEQ ID NO: 135), and a CDR3 comprising an amino acid sequence of GGYDGRGFDY (SEQ ID NO: 136).

In some embodiments, the antigen-binding moiety comprises one, two, or three antigen-binding domains. For example, the antigen-binding moiety may be bispecific and binds to two different antigens selected from the group consisting of HER2, HER3, EGFR, 5T4, FAP alpha, Trop-2, GPC3, VEGFR2, Claudin 18.2, and PD-L1. In some embodiments, the bispecific antigen-binding moiety may bind two different epitopes of the same antigen. For example, the bispecific antibody may bind to two different epitopes of HER2.

### 2. Other Carrier Moieties

Other non-antigen-binding carrier moieties may be used for the present prodrugs. For example, an antibody Fc domain (e.g., a human IgG₁, IgG₂, IgG₃, or IgG₄ Fc), a polymer (e.g., PEG), an albumin (e.g., a human albumin) or a fragment thereof, or a nanoparticle can be used.

By way of example, the IL-15 polypeptide and the Sushi domain and the IL-15 antagonist may be fused to an antibody Fc domain, forming an Fc fusion protein. In some embodiments, the Sushi domain is optionally fused to the C-terminus or N-terminus of one of the heavy chains of the Fc domain, the IL-15 polypeptide is fused to the C-terminus or N-terminus of the Sushi domain through a noncleavable linker, and the masking moiety is fused to the C-terminus or N-terminus of the other heavy domain of the Fc domain through a cleavable peptide or noncleavable linker. In some embodiments, each of the heavy chains of the Fc domain contain mutations that allow their pairing. In some embodiments, mutations may be knobs-into-holes, YTE and/or LALA mutations.

The carrier moiety of the prodrug may comprise an albumin (e.g., human serum albumin) or a fragment thereof. In some embodiments, the albumin or albumin fragment is about 85% or more, about 90% or more, about 91% or more, about 92% or more, about 93% or more, about 94% or more, about 95% or more, about 96% or more, about 97% or more, about 98% or more, about 99% or more, about 99.5% or more, or about 99.8% or more identical to human serum albumin or a fragment thereof.

In some embodiments, the carrier moiety comprises an albumin fragment (e.g., a human serum albumin fragment) that is about 10 or more, 20 or more, 30 or more 40 or more, 50 or more, 60 or more, 70 or more, 80 or more, 90 or more, 100 or more, 120 or more, 140 or more, 160 or more, 180 or more, 200 or more, 250 or more, 300 or more, 350 or more, 400 or more, 450 or more, 500 or more, or 550 or more amino acids in length. In some embodiments, the albumin fragment is between about 10 amino acids and about 584 amino acids in length (such as between about 10 and about 20, about 20 and about 40, about 40 and about 80, about 80 and about 160, about 160 and about 250, about 250 and about 350, about 350 and about 450, or about 450 and about 550 amino acids in length). In some embodiments, the albumin fragment includes the Sudlow I domain or a fragment thereof, or the Sudlow II domain or the fragment thereof.

### D. Linker Components of the Prodrugs

The IL-15 polypeptide and the Sushi domain may be fused to the carrier moiety with or without a peptide linker. The peptide linker may be noncleavable. In some embodiments, the peptide linker is selected from SEQ ID NOs: 11-16. In particular embodiments, the peptide linker comprises the amino acid sequence GGGGSGGGGSGGGGS (SEQ ID NO: 13). In some embodiments, the IL-15 polypeptide (A) is fused to the Sushi domain (S) through a peptide linker. The peptide linker may be at least 25, 30, or 35 amino acids long. In some embodiments, the peptide linker may be 25-45 amino acids. In other embodiments, peptide linker has 25, 26, 27, 28, 29, 30, 31, 32, 33, 34, 35, 36, 37, 38, 39, 40, 41, 42, 43, 44 or 45 amino acids. In some embodiments, the linker comprises an amino acid sequence GSAGSAAGSGEF (SEQ ID NO: 138). In some embodiments, the linker comprises an amino acid sequence (GGGGS)ₙ₁GSAGSAAGSGEF(GGGGS)ₙ₂(SEQ ID NO: 139), wherein n1=1, 2, or 3, and n2=1, 2, or 3. In some embodiments, the linker comprises an amino acid sequence (GGGGS)ₙ₁AA(GGGGS)ₙ₂(SEQ ID NO: 140); wherein n1=2 or 3, and n2=2 or 3.

The masking moiety may be fused to the carrier through a cleavable linker. The cleavable linker may contain one or more (e.g., two or three) cleavable moieties (CM). Each CM may be a substrate for an enzyme or protease selected from legumain, plasmin, TMPRSS-3/4, MMP-2, MMP-9, MT1-MMP, cathepsin, caspase, human neutrophil elastase, beta-secretase, uPA, and PSA. Examples of cleavable linkers include, without limitation, those comprising an amino acid sequence selected from SEQ ID NOs: 17-35, and 36.

In some embodiments, the IL-15 prodrugs of the present disclosure comprise the IL-15 receptor alpha Sushi domain (S), fused to the IL-15 polypeptide through a peptide linker. In certain embodiments, the peptide linker comprises at least 20 amino acids, 25 amino acids, at least 30 amino acids, at least 35 amino acids, or at least 40 amino acids; or 27 amino acids, 32 amino acids, 37 amino acids, 42 amino acids, or 47 amino acids.

### II. Example of IL-15 Prodrugs

In some embodiments, an activatable IL-15 prodrug has a molecular structure illustrated in any one of FIGS. 1A-1C and FIGS. 2A-2C. In a particular embodiment, the IL-15 prodrug has a molecular structure illustrated in any one of FIGS. 1B or FIG. 2B. In some embodiments, the IL-15 prodrug comprises a structure illustrated in any one of FIGS. 3A-3C. In a particular embodiment, the IL-15 prodrug comprises a structure illustrated in FIG. 3B. In some embodiments, the carrier moiety is an antibody that comprises one antigen-binding moiety, as illustrated in FIGS. 4A, 4B, 5A, or 5B. In a preferred embodiment, the IL-15 prodrug comprises a structure selected from FIG. 4B and FIG. 5B.

The IL-15 prodrug may not contain the Sushi domain or any of its functional analogs. In some embodiments, the IL-15 prodrug comprises an IL-15 polypeptide comprising one or more mutations at a position or positions selected from E46, V49, L45, S51, and L52 (numbering according to SEQ ID NO: 2). In some embodiments, the IL-15 polypeptide comprises the mutation E46K (numbering according to SEQ ID NO: 2). In other embodiments, the IL-15 polypeptide comprises the mutations E46K/N65D (numbering according to SEQ ID NO: 2). In yet other embodiments, IL-15 polypeptide comprises the mutations E46K/Q108E (numbering according to SEQ ID NO: 2).

In some embodiments, an IL-15 prodrug of the present disclosure comprises an IgG₁ Fc domain as the carrier moiety. For example, the IL-15 prodrug may be selected from Table 2. In other embodiments, an IL-15 prodrug of the present invention comprises an IgG₄ Fc domain. For example, the IL-15 prodrug may be selected from Table 3. In some embodiments, an IL-15 prodrug of the present invention comprises an antibody that binds to human PD-L1 as the carrier moiety. For example, the IL-15 prodrug may be selected from Table 4. In some embodiments, an IL-15 prodrug of the present invention comprises an antibody that binds to human PD-1 as the carrier moiety. For example, the IL-15 prodrug may be selected from Table 5.

TABLE 2
Examples of activatable IgG₁ Fc-IL-15 fusion polypeptides

| | Fc fused with IL-15 | Fc fused with the |
|---|---|---|
| Name | or its analog | masking moiety |
| IgG1 Fc-IL-15 Fusion A | SEQ ID NO: 38 | SEQ ID NO: 37 |
| IgG1 Fc-IL-15 Fusion B | SEQ ID NO: 39 | SEQ ID NO: 37 |
| IgG1 Fc-IL-15 Fusion C | SEQ ID NO: 39 | SEQ ID NO: 70, 71, |
| IgG1 Fc-IL-15 Fusion D | SEQ ID NO: 75 | 72, 73, or 74 |
| IgG1 Fc-IL-15 Fusion E | SEQ ID NO: 76 | |
| IgG1 Fc-IL-15 Fusion F | SEQ ID NO: 77 | |
| IgG1 Fc-IL-15 Fusion G | SEQ ID NO: 78 | |
| IgG1 Fc-IL-15 Fusion H | SEQ ID NO: 79 | |

TABLE 3
Examples of activatable IgG₄ Fc-IL-15 fusion polypeptides

| | Fc fused with IL-15 | Fc fused with the |
|---|---|---|
| Name | or its analog | masking moiety |
| IgG4 Fc-IL-15 Fusion A | SEQ ID NO: 82 | SEQ ID NO: 80 or 87 |
| IgG4 Fc-IL-15 Fusion B | SEQ ID NO: 83 | SEQ ID NO: 80 or 87 |
| IgG4 Fc-IL-15 Fusion C | SEQ ID NO: 84 | SEQ ID NO: 80 or 87 |
| IgG4 Fc-IL-15 Fusion D | SEQ ID NO: 85 | SEQ ID NO: 80 or 87 |
| IgG4 Fc-IL-15 Fusion E | SEQ ID NO: 86 | SEQ ID NO: 80 or 87 |
| IgG4 Fc-IL-15 Fusion F | SEQ ID NO: 82 | SEQ ID NO: 81 |
| IgG4 Fc-IL-15 Fusion G | SEQ ID NO: 83 | SEQ ID NO: 81 |
| IgG4 Fc-IL-15 Fusion H | SEQ ID NO: 84 | SEQ ID NO: 81 |
| IgG4 Fc-IL-15 Fusion I | SEQ ID NO: 85 | SEQ ID NO: 81 |
| IgG4 Fc-IL-15 Fusion J | SEQ ID NO: 86 | SEQ ID NO: 81 |

TABLE 4
Examples of activatable PD-L1 antibody/IL-15 fusion polypeptides

| | HC Polypeptide | HC Polypeptide | |
|---|---|---|---|
| | Chain fused with | Chain fused with | |
| Name | IL-15 or its analog | the masking moiety | Light Chain |
| PDL1 antibody-IL-15 | SEQ ID NO: 45 | SEQ ID NO: 47 | SEQ ID NO: |
| Fusion A | | | 50 or 51 |
| PDL1 antibody-IL-15 | SEQ ID NO: 46 | SEQ ID NO: 47 | SEQ ID NO: |
| Fusion B | | | 50 or 51 |
| PDL1 antibody-IL-15 | SEQ ID NO: 45 | SEQ ID NO: 48 | SEQ ID NO: |
| Fusion C | | | 50 or 51 |
| PDL1 antibody-IL-15 | SEQ ID NO: 45 | SEQ ID NO: 49 | SEQ ID NO: |
| Fusion D | | | 50 or 51 |

TABLE 5
Examples of activatable PD-1 antibody-IL-15 fusion polypeptides

| | HC fused with IL-15 | HC fused with the | | |
|---|---|---|---|---|
| Name | or its analog | masking moiety | Light Chain | Comments |
| PD1 antibody- | SEQ ID NO: 52 | SEQ ID NO: 54 | SEQ ID NO: | Masked with |
| IL-15 Fusion A | | | 55 or 56 | IL-2Rβ ECD |
| PD1 antibody- | SEQ ID NO: 53 | SEQ ID NO: 54 | SEQ ID NO: | |
| IL-15 Fusion B | | | 55 or 56 | |
| PD1 antibody- | SEQ ID NO: 52 | SEQ ID NO: | SEQ ID NO: | Masked with |
| IL-15 Fusion C | | 60 or 61 | 55 or 56 | scFv1 or scFv2 |
| PD1 antibody- | SEQ ID NO: 58 | SEQ ID NO: | SEQ ID NO: | |
| IL-15 Fusion D | | 60 or 61 | 55 or 56 | |
| PD1 antibody- | SEQ ID NO: 59 | SEQ ID NO: | SEQ ID NO: | |
| IL-15 Fusion E | | 60 or 61 | 55 or 56 | |
| PD1 antibody- | SEQ ID NO: 62 | SEQ ID NO: 61 | SEQ ID NO: 55 | IL-15 with E46K, |
| IL-15 Fusion F | | | | no Sushi |
| PD1 antibody- | SEQ ID NO: 63 | SEQ ID NO: 61 | SEQ ID NO: 55 | IL-15 with E46K/ |
| IL-15 Fusion G | | | | N65D, no Sushi |
| PD1 antibody- | SEQ ID NO: 69 | SEQ ID NO: 61 | SEQ ID NO: 55 | Long linker between |
| IL-15 Fusion H | | | | Sushi and IL-15 mutein |
| PD1 antibody- | SEQ ID NO: 64 | SEQ ID NO: 66 | SEQ ID NO: 55 | Fc domains are |
| IL-15 Fusion I | | | | identical; no Fc |
| PD1 antibody- | SEQ ID NO: 65 | SEQ ID NO: 66 | SEQ ID NO: 55 | mutations to promote |
| IL-15 Fusion J | | | | heterodimerization |
| PD1 antibody- | SEQ ID NO: 67 | SEQ ID NO: 66 | SEQ ID NO: 55 | |
| IL-15 Fusion K | | | | |
| PD1 antibody- | SEQ ID NO: 68 | SEQ ID NO: 66 | SEQ ID NO: 55 | |
| IL-15 Fusion L | | | | |

Specific, nonlimiting examples of IL-15 polypeptides, Sushi domains, cytokine antagonists/masks, carriers, peptide linkers, and prodrugs are shown in the Sequences section below. Further, the prodrugs of the present disclosure may be made by well-known recombinant technology. For examples, one more expression vectors comprising the coding sequences for the polypeptide chains of the prodrugs may be transfected into mammalian host cells (e.g., CHO cells), and cells are cultured under conditions that allow the expression of the coding sequences and the assembly of the expressed polypeptides into the prodrug complex. In order for the prodrug to remain inactive, the host cells that express no or little uPA, MMP-2 and/or MMP-9 may be used. In some embodiments, the host cells may contain null mutations (knockout) of the genes for these proteases.

### III. Pharmaceutical Compositions

Pharmaceutical compositions comprising the prodrugs and muteins (i.e., the active pharmaceutical ingredient or API) of the present disclosure may be prepared by mixing the API having the desired degree of purity with one or more optional pharmaceutically acceptable excipients (see, e.g., Remington's Pharmaceutical Sciences, 16th Edition., Osol, A. Ed. (1980)) in the form of lyophilized formulations or aqueous solutions. Pharmaceutically acceptable excipients (or carriers) are generally nontoxic to recipients at the dosages and concentrations employed, and include, but are not limited to: buffers containing, for example, phosphate, citrate, succinate, histidine, acetate, or another inorganic or organic acid or salt thereof; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including sucrose, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof, such as citrate, phosphate, succinate, tartrate, fumarate, gluconate, oxalate, lactate, and acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter- and intra-molecular interactions. Tonicity agents can be present in any amount between 0.1% to 25% by weight, or more preferably between 1% to 5% by weight, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

The choice of pharmaceutical carrier, excipient or diluent may be selected with regard to the intended route of administration and standard pharmaceutical practice. Pharmaceutical compositions may additionally comprise any suitable binder(s), lubricant(s), suspending agent(s), coating agent(s) or solubilizing agent(s).

There may be different composition/formulation requirements dependent on the different delivery systems. By way of example, pharmaceutical compositions useful in the present invention may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route.

In some embodiments, the pharmaceutical composition of the present disclosure is a lyophilized protein formulation. In other embodiments, the pharmaceutical composition may be an aqueous liquid formulation.

### IV. Methods of Treatment

The IL-15 prodrug can be used to treat a disease, depending on the antigen bound by the antigen-binding domain. In some embodiments, the IL-15 prodrug is used to treat cancer. In some embodiments, the IL-15 prodrug is used to treat an infection, for example when the drug molecule is an antibacterial agent or an antiviral agent.

In some embodiments, a method of treating a disease (such as cancer, a viral infection, or a bacterial infection) in a subject comprises administering to the subject an effective amount of an IL-15 prodrug. In other embodiments, the method of treatment further comprises administering an additional therapeutic agent in combination with (before, after, or concurrently with) the IL-15 prodrug. The additional agent may be an antibody or fragment thereof, small-molecule drug, or other type of therapeutic drug, some of which are disclosed herein.

In some embodiments, the cancer is a solid cancer. In some embodiments, the cancer is a blood cancer or a solid tumor. Exemplary cancers that may be treated include, but are not limited to, leukemia, lymphoma, kidney cancer, bladder cancer, urinary tract cancer, cervical cancer, brain cancer, head and neck cancer, skin cancer, uterine cancer, testicular cancer, esophageal cancer, liver cancer, colorectal cancer, stomach cancer, squamous cell carcinoma, prostate cancer, pancreatic cancer, lung cancer such as non-small cell lung cancer, cholangiocarcinoma, breast cancer, and ovarian cancer.

In some embodiments, the IL-15 prodrug is used to treat a bacterial infection such as sepsis. In some embodiments, the bacteria causing the bacterial infection are drug-resistant bacteria. In some embodiments, the antigen-binding moiety binds to a bacterial antigen.

In some embodiments, the IL-15 prodrug is used to treat a viral infection. In some embodiments, the virus causing the viral infection is hepatitis C (HCV), hepatitis B (HBV), human immunodeficiency virus (HIV), a human papilloma virus (HPV). In some embodiments, the antigen-binding moiety binds to a viral antigen.

Generally, dosages, and routes of administration of the present pharmaceutical compositions are determined according to the size and conditions of the subject, according to standard pharmaceutical practice. In some embodiments, the pharmaceutical composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, intracranially, or intraspinally. In some embodiments, the composition is administered to a subject intravenously.

In some embodiments, the dosage of the pharmaceutical composition is a single dose or a repeated dose. In some embodiments, the doses are given to a subject once per day, twice per day, three times per day, or four or more times per day. In some embodiments, about 1 or more (such as about 2, 3, 4, 5, 6, or 7 or more) doses are given in a week. In some embodiments, the pharmaceutical composition is administered weekly, once every 2 weeks, once every 3 weeks, once every 4 weeks, weekly for two weeks out of 3 weeks, or weekly for 3 weeks out of 4 weeks. In some embodiments, multiple doses are given over the course of days, weeks, months, or years. In some embodiments, a course of treatment is about 1 or more doses (such as about 2, 3, 4, 5, 7, 10, 15, or 20 or more doses).

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It is understood that aspects and variations of the invention described herein include “consisting” and/or “consisting essentially of” aspects and variations. All publications and other references mentioned herein are incorporated by reference in their entirety. Although a number of documents are cited herein, this citation does not constitute an admission that any of these documents forms part of the common general knowledge in the art.

### EXAMPLES

### Transient Transfection of HEK293 Cells

Expression plasmids were co-transfected into 3×10⁶ cell/ml freestyle HEK293 cells at 2.5-3 μg/ml using polyethylenimine (PEI). For Fc-based IL-15 prodrugs, the Fc-IL-15 mutein fusion polypeptide and the Fc-masking moiety fusion polypeptide were in a 1:2 ratio. For antibody-based IL-15 prodrugs, the knob heavy chain (containing IL-15 polypeptide), hole heavy chain (containing the masking moiety), and the light chain DNA were in a 2:1:2 molar ratio. The cell cultures were harvested 6 days after transfection by centrifuging at 9,000 rpm for 45 min followed by 0.22 μM filtration.

### Protein Purification

The Fc- and antibody-based IL-15 fusion polypeptides were, in general, purified by Protein A affinity chromatography followed by ion exchange chromatography, hydrophobic interaction chromatography, and/or size exclusion chromatography. In some cases, the purifications of the proteins of the antibody-based IL-15 prodrugs were carried out by using four steps of chromatography, including: 1) Protein A affinity chromatography; 2) Capto™ Adhere operated in a flow-through mode; 3) Capto™ MMC ImpRes, and 4) Q Sepharose® HP operated in a flow-through mode. Capto™ Adhere was equilibrated by the buffer containing 50 mM acetic acid, 30 mM NaCl (pH 5.5). Capto™ MMC ImpRes was equilibrated using the buffer A (50 mM acetic acid, 30 mM NaCl, pH 5.5) and eluted using a 30 CV linear gradient with buffer B (50 mM acetic acid, 0.5 M Arginine, pH 5.5). Q Sepharose® HP was equilibrated with 40 mM Bis Tris, pH 6.5.

### SEC-HPLC Analysis

SEC-HPLC was carried out using an Agilent 1100 Series HPLC system with a TSKgel® G3000SWXL column (7.8 mmIDX 30cm, 5 μm particle size) from Tosoh Bioscience. A sample of up to 100 μl was loaded. The column was run with a buffer containing 200 mM K₃PO₄, 250 mM KCl, pH 6.5. The flow rate was 0.5 ml/min. The column was run at room temperature. The protein elution was monitored both at 220 nm and 280 nm.

### SDS-PAGE Analysis

10 μl of the culture supernatants or 20 μg of purified protein samples were mixed with Bolt™ LDS Sample Buffer (Novex) with or without reduce reagents. The samples were heated at 70° C. for 3 min and then loaded to a NuPAGE™4-12% BisTris Gel (Invitrogen). The gel was run in NuPAGE™ MOPS SDS Running buffer (Invitrogen) at 200 Volts for 40 min and then stained with Coomassie.

### Proteolytic Treatment

One μg of the protease, human MMP-2 (R&D systems), human MMP-9 (R&D systems), mouse MMP-2 (R&D systems), or mouse MMP-9 (R&D systems) was added to 50 μg of the precursor protein, and incubated at 37° C. overnight.

### CTLL2 Assay

CTLL2 cells were grown in the RPMI 1640 medium supplemented with L-glutamine, 10% fetal bovine serum, 10% non-essential amino acids, 10% sodium pyruvate, and 55 μM beta-mercaptoethanol. CTLL2 cells were non-adherent and maintained at 5×10⁴-1×10⁶ cells/ml in medium with 100 ng/ml of IL-15. Generally, cells were split twice per week. For bioassays, it was best to use cells no less than 48 hours after passage.

Samples were diluted at 2× concentration in 50 μl/well in a 96 well plate. The IL-15 standards were titrated from 20 ng/ml (2× concentration) to 3× serial dilutions for 12 wells. Samples were titer tested as appropriate. CTLL2 cells were washed 5 times to remove IL-15, dispensed 5000 cells/well in 50 μl and cultured overnight or for at least 18 hours with the samples. Subsequently, 100 μl/well Cell Titer Glo reagents (Promega) were added and luminescence was measured.

### NK92 Proliferation Assay

NK92 cell proliferation assays were also carried out, according to the protocols below.

The NK92 cell line is a factor dependent cell line that requires IL-2 for growth and survival. Prior to assay, the cells are washed to remove IL-2 and cultured overnight without growth factor. Cells are harvested and washed again to remove residual growth factor. Cells (20,000/well) are then added to 96 well plates containing serial dilution of test articles and controls. Plates are incubated overnight, and Cell Titer Glo (Promega) is added and luminescence measured. This provides a measure of ATP levels as an indicator of cell viability.

The assays were carried out using several IL-15 prodrugs masked with IL-2Rμ extra-cellular domain (ECD), IL-2Rβ ECD and IL-2Rγ ECD, and scFv molecules derived from the IL-15 antibody 146B7.

### pSTAT5 Analysis

NK92/pSTAT5 stable cell line were starved in RPMI 1640 medium supplemented with 0.1% FBS overnight. 5×10⁵ of cells were seeded in each well of a 96-well plate prior to incubation at 37° C. and 5% CO₂ overnight. IL-15 fusion polypeptides were added to the cells and incubated for 5-6 hours in the incubator. Subsequently, 100 μl of Pierce™ Firefly Luc One-Step Glow Assay solution was added and the bioluminescent were read using a luminometer.

### Enzyme-Linked Immunosorbent Assay (ELISA)

10 μg/ml of IL-15 fusion proteins in PBS were seeded to the 96-well plate at 100 μl/well and coated at 4 degree for overnight. The wells were washed by PBS three times and blocked with 100 μl 2% milk/PBS for 1 hr. The wells were then washed three times by PBS and 100 μl protein samples with 3-fold serial dilution were added for 1 hr incubation at room temperature (RT). After three times of PBS washing, 100 μl of HRP conjugated anti-IgG antibody was added and incubated at RT for 1 hr. Subsequently, the wells were washed again 3 times using PBS, followed by the addition of detection reagents and measurement of optical density (OD) at 450 nM.

### Example 1: Expression and Testing of IL-15 Prodrugs

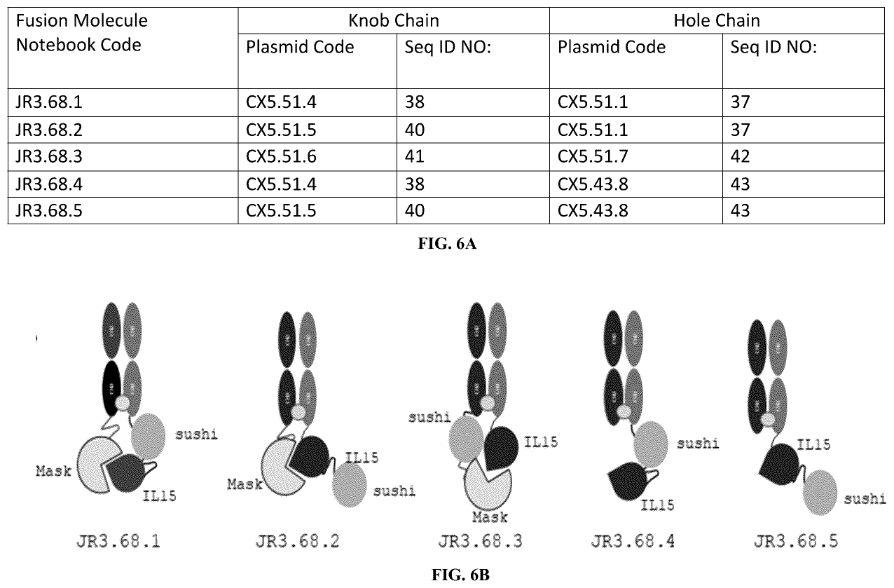

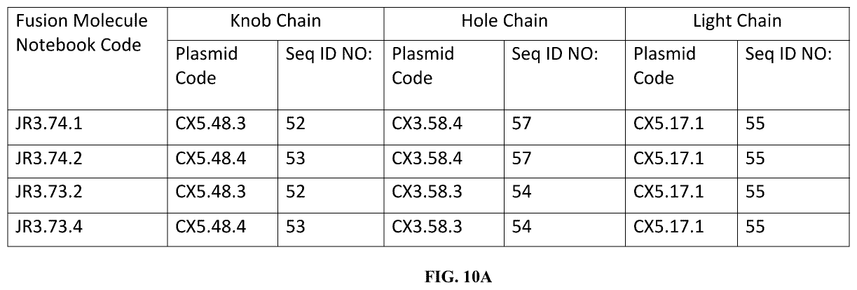

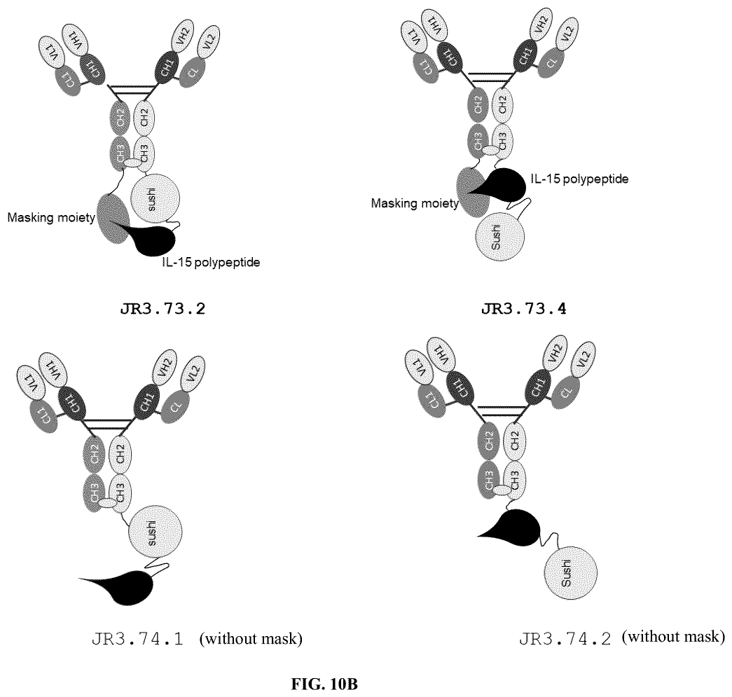

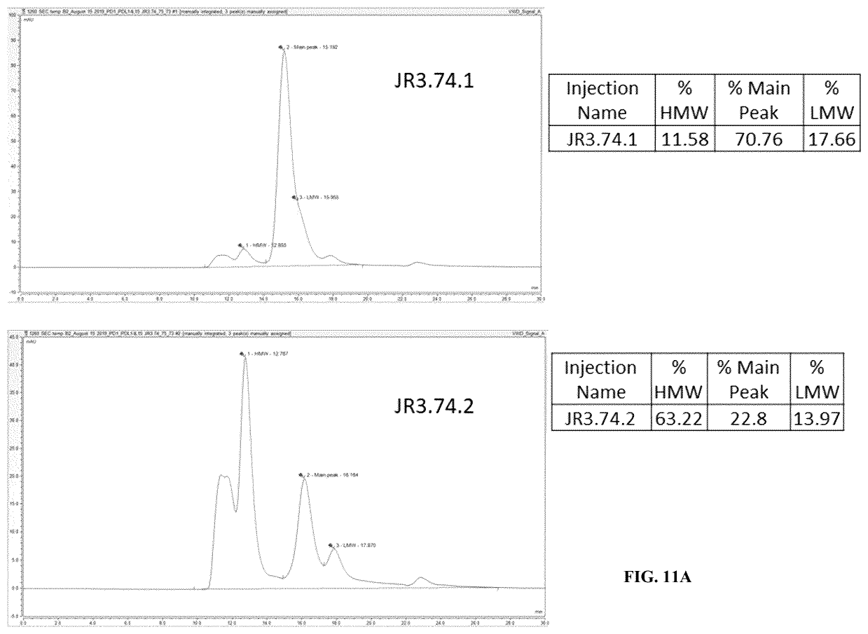

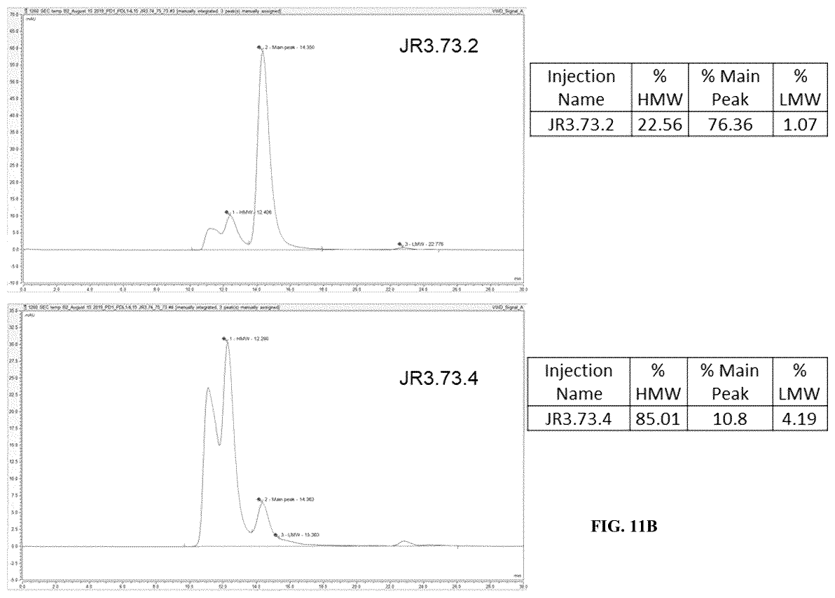

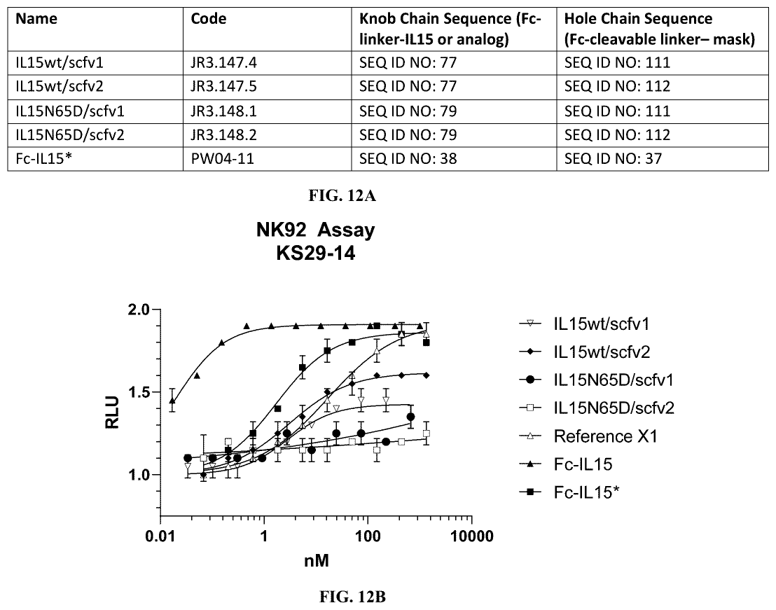

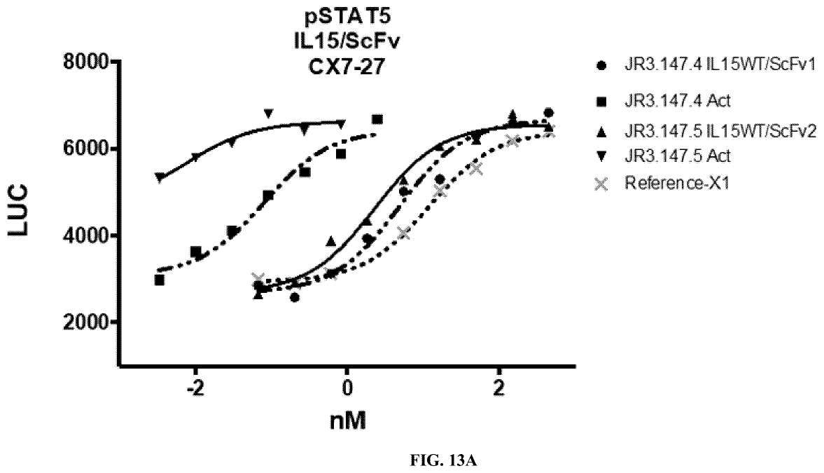

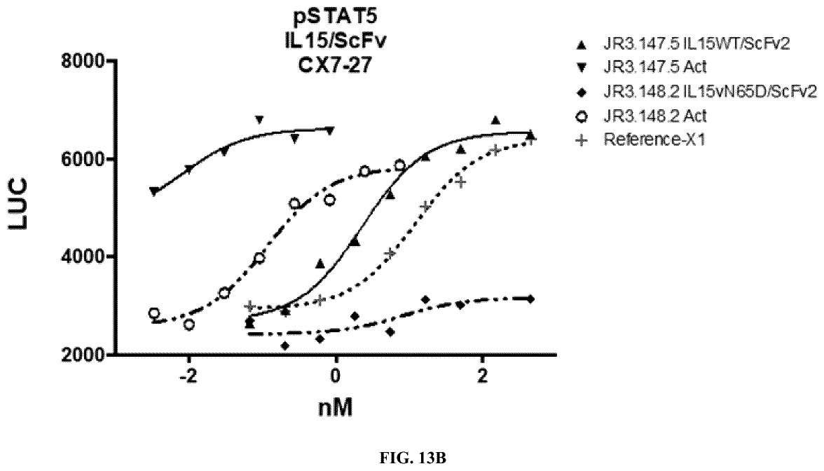

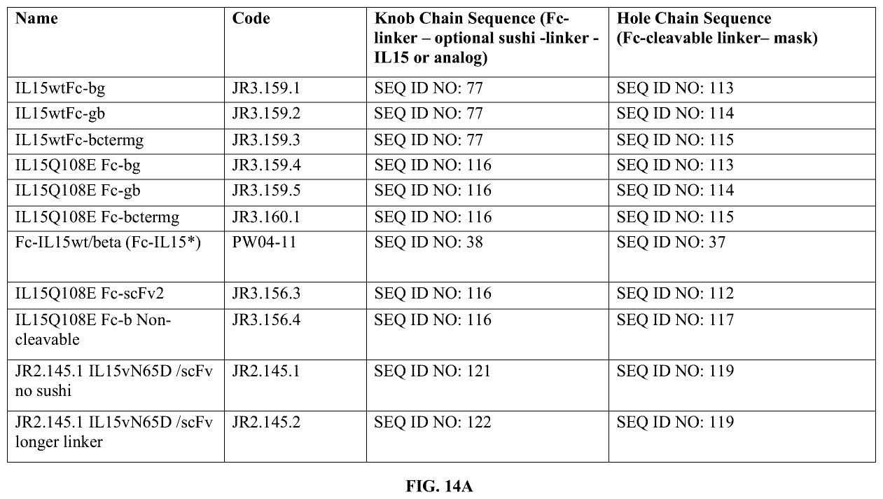

A number of the prodrugs were constructed and recombinantly expressed in HEK293 cells (see FIG. 6A and FIG. 10A). In the IL-15 prodrugs, IL-15 polypeptides were expressed as part of a fusion polypeptide and tested for their biological activities. Some of the sequences of the IL-15 fusion polypeptides expressed are listed in FIG. 6A, FIG. 10A, and FIG. 12A.

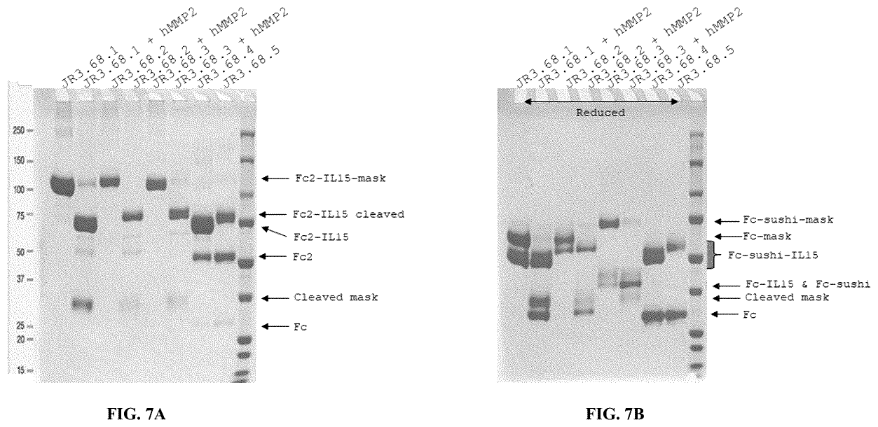

The expressed IL-15 fusion polypeptides were tested by SDS-PAGE prior to and after activation (FIG. 7A; non-reduced; and FIG. 7B; reduced). The data shows that the masking moieties of JR3.68.1, JR3.68.2, and JR3.68.3 samples were successfully cleaved by the protease treatment.

### Example 2: Purification of Activatable IL-15 Prodrug Components

Activatable IL-15 prodrugs JR3.68.1, JR3.68.2 and JR3.68.3 were purified via Protein A column and analyzed using SEC-HPLC. JR3.68.1 (FIG. 1A) has a Sushi domain fused via a peptide linker to the C-terminus of one of the heavy chains of the Fc domain, the IL-15 polypeptide is fused to the C-terminus of the Sushi domain through a peptide linker, and the masking moiety (IL2Rβ ECD) is fused to the C-terminus of the other heavy chain of the Fc domain. JR3.68.2 is illustrated on FIG. 1B, and JR3.68.3 is illustrated on FIG. 1C.

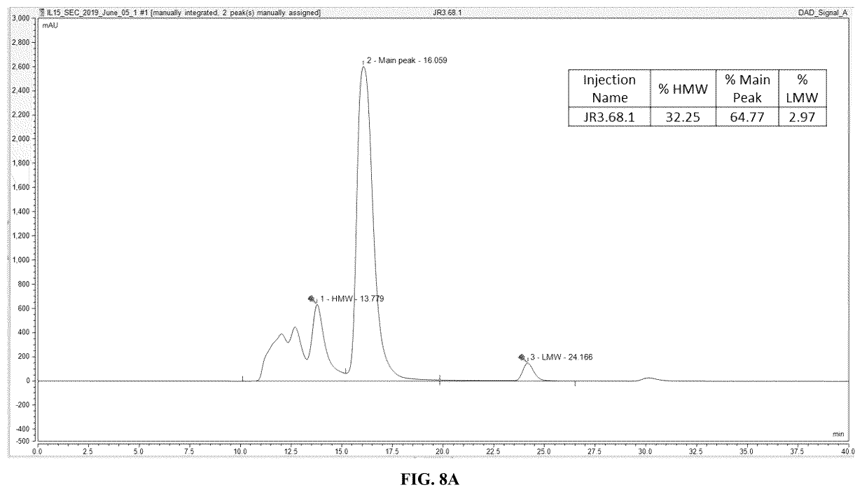

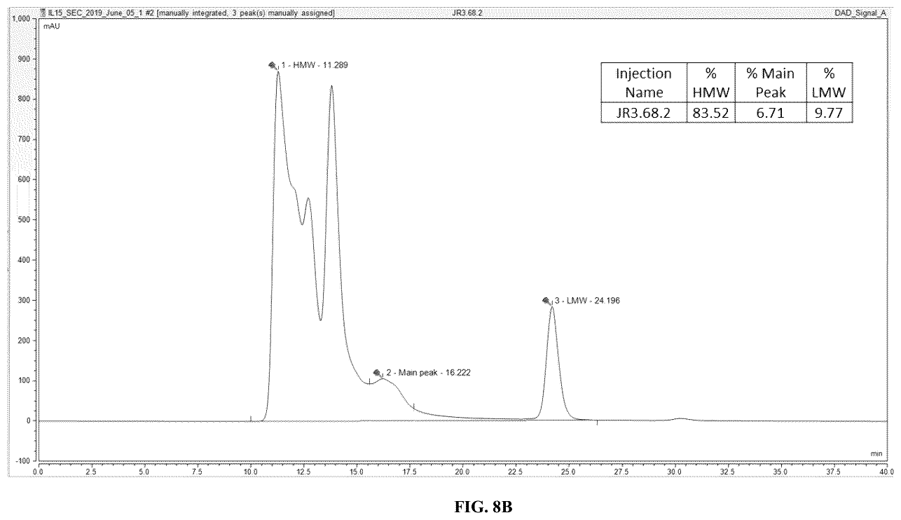

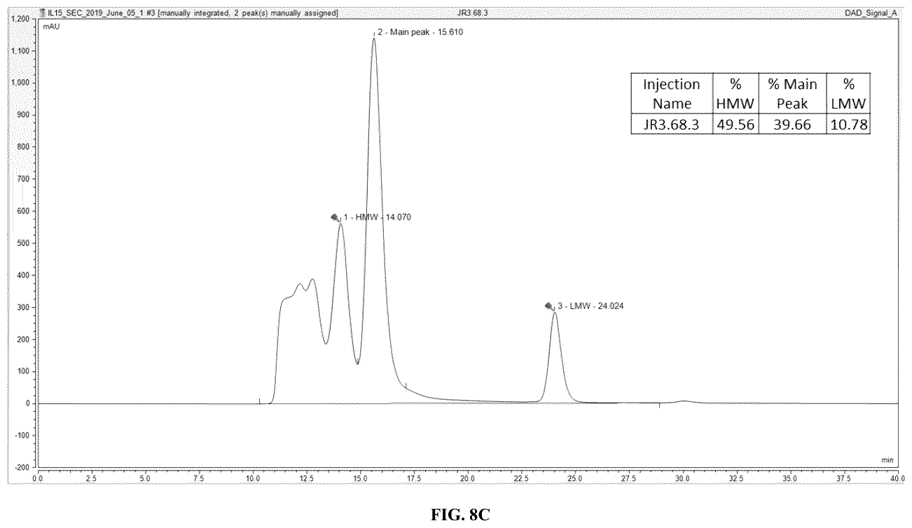

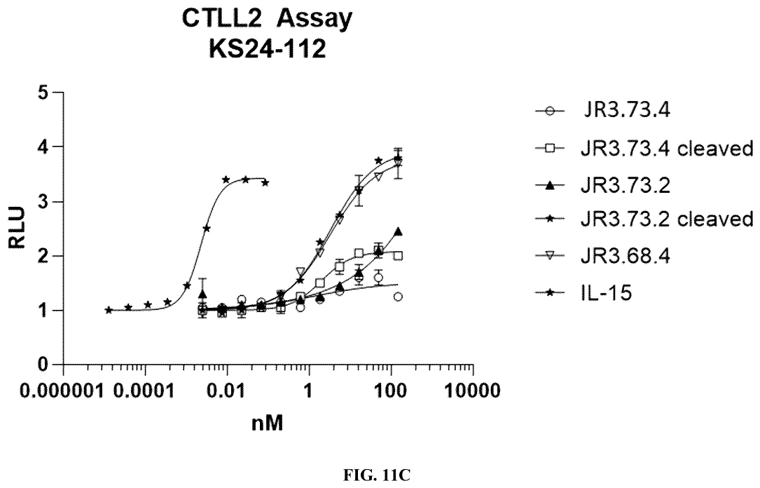

It was surprising that the format, arrangement, relative location or configuration of the several components of the prodrug molecule had significant effects on the levels of drug aggregates, when purified by Protein A affinity column. It was clear that the format of Fc-Sushi-IL-15 (comprising two polypeptide chains SEQ ID NO: 37 and SEQ ID NO: 38) (JR3.68.1) had a significantly higher purity (as evidenced by the higher main peak; FIG. 8A) and lower level of aggregation when compared to the format of Fc-IL-15-Sushi (JR3.68.2, having two polypeptide chains of SEQ ID NO: 37 and SEQ ID NO: 40) (FIG. 8B). Meanwhile, the format where the Sushi domain and the cytokine were on the different heavy chains of the Fc domain had a SEC-HPLC main peak purity better than JR3.68.2 (JR3.68.3, FIG. 8C) but lower than that of JR3.68.1 (FIG. 8A). The trend was essentially the same when the carrier was an antibody (e.g., nivolumab, an antibody against human PD-1; FIG. 11B, JR3.73.2 vs. JR3.73.4).

We also unexpectedly observed that by adding a masking moiety, the purities of the fusion polypeptides were significantly enhanced. We observed that the JR3.73.2 IL-15 prodrug with an antibody as a carrier moiety appeared to have a higher monomer purity by SEC-HPLC than the activated version JR3.74.1) (FIG. 11A and FIG. 11B). We also observed that the monomer peak of JR3.74.1 had a significant shoulder (FIG. 11A), which may indicate potential challenge of further purification.

### Example 3: Cell-based Activities of IL-15 Prodrugs

### CTLL2 Assay

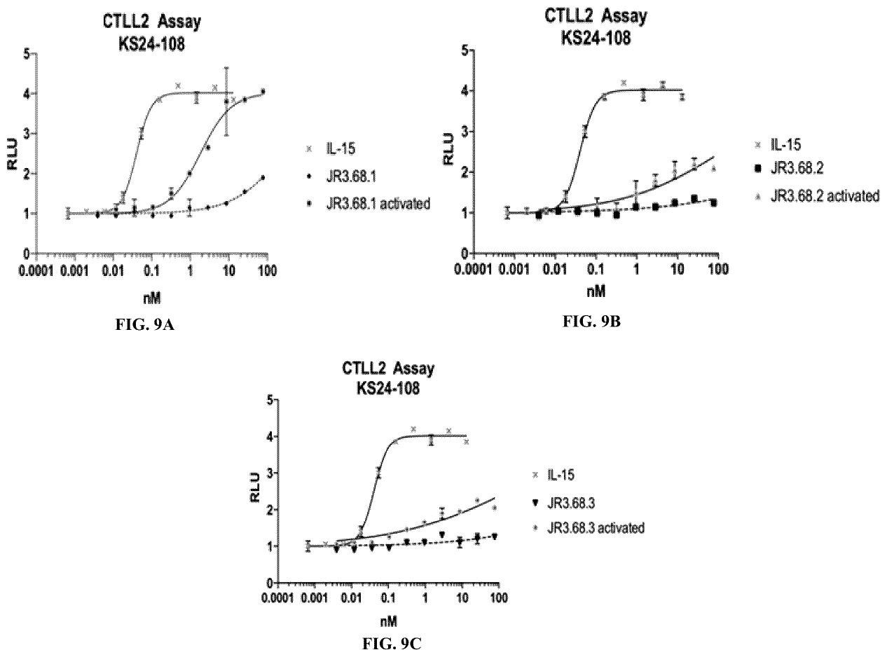

The CTLL2 cell-based activities of the IL-15 prodrugs JR3.68.1, JR3.68.2, and JR3.68.3 were determined before and after activation, as shown in FIGS. 9A-9C. The results show that JR3.68.1 had significant activation after protease treatment. The cell-based activities of the IL-15 prodrugs with an antibody as a carrier moiety are shown in FIG. 11C. The results show that the IL-15 prodrug JR3.73.2 was activatable.

### NK92 Assay

NK92 cell proliferation assays were also carried out for several IL-15 prodrugs masked with scFv molecules (derived from the IL-15 antibody 146B7), IL-2Rβ ECD, or IL-2Rβ ECD and IL-2Rγ ECD. The NK92 proliferation assay results of the IL-15 prodrugs that are masked with scFv1 or scFv2 of IL-15 antibody 146B7 show that both scFv2 and scFv1 significantly masked the activity of the IL-15 WT and IL-15 mutein with N65D mutation (FIG. 12B).

The NK92 cell-based activities of the activatable IL-15 fusion polypeptides prior to and after activation was determined using the pSTAT5 method. FIG. 13A shows that both scFv2 and scFv1 masked the wild type IL-15 to the similar extent, and the fusion polypeptides were activatable upon protease treatment. FIG. 13B shows that scFv2 significantly masked the activity of the IL-15 mutein N65D. The results also demonstrate that scFv1 efficiently masked the IL-15 mutein. It was unexpected that both IL-15 prodrugs were activatable in vitro upon protease treatment but without further purification to remove the cleaved scFv molecules. It was also surprising that scFv2 had significantly stronger masking effect than that of scFv1 for IL-15 mutein N65D.

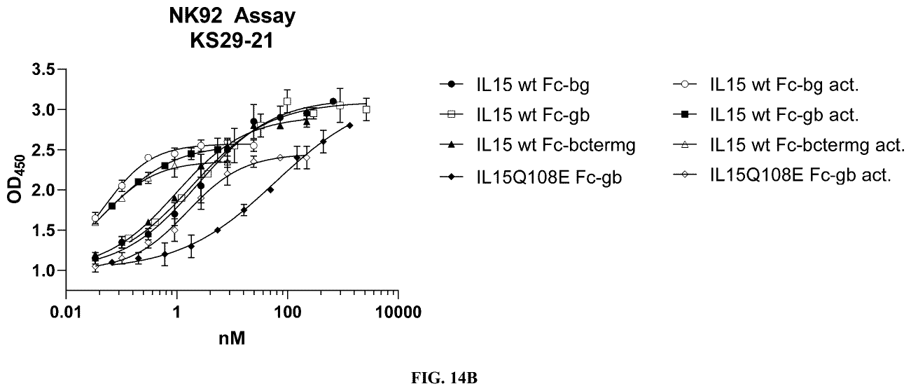

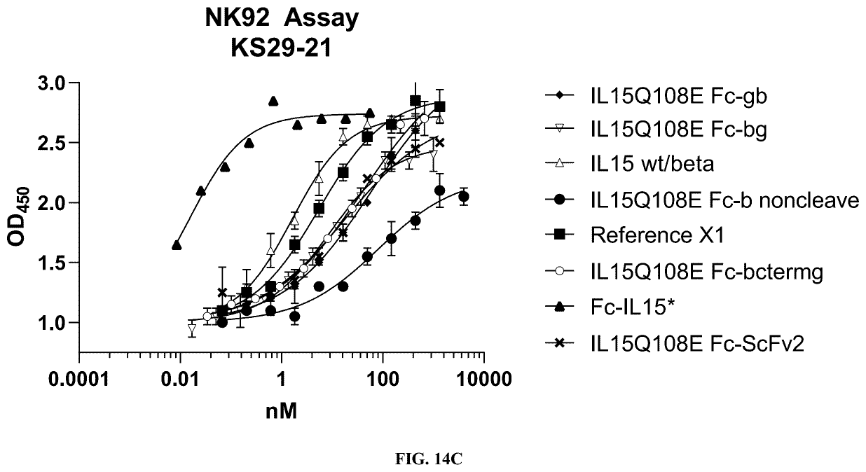

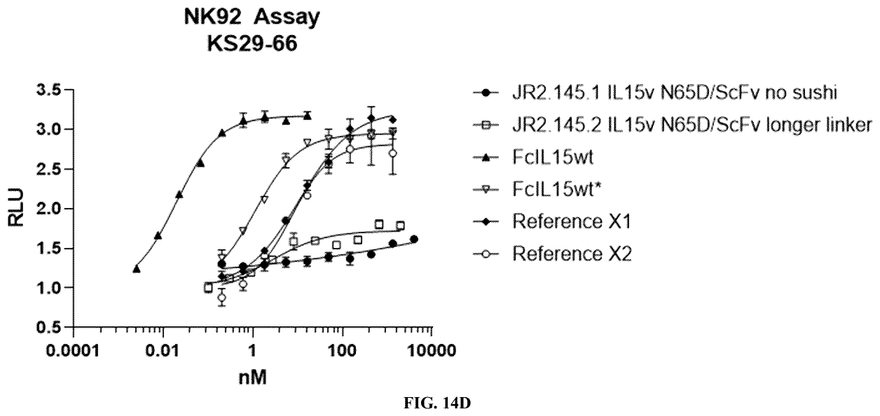

The NK92 cell-based activities of additional activatable IL-15 fusion polypeptides masked with IL-2Rβ ECD or IL-2Rβ ECD and IL-2Rγ ECD were determined. In these fusion polypeptides, wild type IL-15 was masked with IL-2Rβ ECD and IL-2RγECD. The results show that IL-2Rβ ECD in combination with IL-2RγECD formed an effective mask for the wild type IL-15 and that the IL-15 prodrugs were activatable upon protease treatment (FIG. 14A). The activity of IL-15 mutein Q108E (which was activatable upon protease treatment) was also masked with IL-2Rβ ECD and IL-2RγECD (FIG. 14C).

We also determined the NK92 cell-based assay results of the activatable Fc-IL-15 fusion polypeptide without a Sushi domain (JR2.145.1) and one with a longer linker between the Sushi domain and the IL-15 polypeptide moiety (JR2.145.2). The data showed significant masking of the IL-15 mutein N65D in both cases. The results indicate that the scFv2 mask was effective in masking IL-15 polypeptide in the absence of the Sushi domain. The masking domain also worked well when the linker between the Sushi domain and the IL-15 polypeptide was longer (32 amino acids).

The above non-limiting examples are provided for illustrative purposes only in order to facilitate a more complete understanding of the disclosed subject matter. These examples should not be construed to limit any of the embodiments described in the present specification, including those pertaining to the antibodies, pharmaceutical compositions, or methods and uses for treating cancer, a neurodegenerative or an infectious disease.

### SEQUENCES

In the sequences below, boxed residues indicate mutations. Underlines in cleavable linkers indicate protease substrate sequences.

Human IL-2
SEQ ID NO: 1
APTSSSTKKT QLQLEHLLLD LQMILNGINN YKNPKLTRML TFKFYMPKKA TELKHLQCLE
EELKPLEEVL NLAQSKNFHL RPRDLISNIN VIVLELKGSE TTFMCEYADE TATIVEFLNR
WITFCQSIIS TLT
Human IL-15
SEQ ID NO: 2
NWVNVISDLK KIEDLIQSMH IDATLYTESD VHPSCKVTAM KCFLLELQVI SLESGDASIH
DTVENLIILA NNSLSSNGNV TESGCKECEE LEEKNIKEFL QSFVHIVQMF INT
Human IL-2 Receptor Beta Subunit Extracellular Domain
(uniprot/P14784)
SEQ ID NO: 3
AVNGTSQFTC FYNSRANISC VWSQDGALQD TSCQVHAWPD RRRWNQTCEL LPVSQASWAC
NLILGAPDSQ KLTTVDIVTL RVLCREGVRW RVMAIQDFKP FENLRLMAPI SLQVVHVETH
RCNISWEISQ ASHYFERHLE FEARTLSPGH TWEEAPLLTL KQKQEWICLE TLTPDTQYEF
QVRVKPLQGE FTTWSPWSQP LAFRTKPAAL GKDT
Human IL-2 Receptor Beta Subunit Extracellular Domain
Mutant D68E (uniprot/P14784)
SEQ ID NO: 4
AVNGTSQFTC FYNSRANISC VWSQDGALQD TSCQVHAWPD RRRWNQTCEL LPVSQASWAC
NLILGAPESQ KLTTVDIVTL RVLCREGVRW RVMAIQDFKP FENLRLMAPI SLQVVHVETH
RCNISWEISQ ASHYFERHLE FEARTLSPGH TWEEAPLLTL KQKQEWICLE TLTPDTQYEF
QVRVKPLQGE FTTWSPWSQP LAFRTKPAAL GKDT
Human IL-2 Receptor Beta Subunit Extracellular Domain
Mutant E136Q/H138R (uniprot/P14784)
SEQ ID NO: 5
AVNGTSQFTC FYNSRANISC VWSQDGALQD TSCQVHAWPD RRRWNQTCEL LPVSQASWAC
NLILGAPDSQ KLTTVDIVTL RVLCREGVRW RVMAIQDFKP FENLRLMAPI SLQVVHVETH
RCNISWEISQ ASHYFQRRLE FEARTLSPGH TWEEAPLLTL KQKQEWICLE TLTPDTQYEF
QVRVKPLQGE FTTWSPWSQP LAFRTKPAAL GKDT
Human IL-2 Receptor Gamma Subunit Extracellular Domain
(uniprot/P31785)
SEQ ID NO: 6
LNTTILTPNG NEDTTADFFL TTMPTDSLSV STLPLPEVQC FVFNVEYMNC TWNSSSEPQP
TNLTLHYWYK NSDNDKVQKC SHYLFSEEIT SGCQLQKKEI HLYQTFVVQL QDPREPRRQA
TQMLKLQNLV IPWAPENLTL HKLSESQLEL NWNNRFLNHC LEHLVQYRTD WDHSWTEQSV
DYRHKFSLPS VDGQKRYTFR VRSRFNPLCG SAQHWSEWSH PIHWGSNTSK ENPFLFALEA
IL-15 receptor alpha subunit Sushi domain
SEQ ID NO: 7
ITCPPPMSVE HADIWVKSYS LYSRERYICN SGFKRKAGTS SLTECVLNKA TNVAHWTTPS
LKCIRDPALV HQRPA
Amino acid sequence of IL-15 receptor alpha
SEQ ID NO: 8
MAPRRARGCR TLGLPALLLL LLLRPPATRG ITCPPPMSVE HADIWVKSYS LYSRERYICN
SGFKRKAGTS SLTECVLNKA TNVAHWTTPS LKCIRDPALV HQRPAPPSTV TTAGVTPQPE
SLSPSGKEPA ASSPSSNNTA ATTAAIVPGS QLMPSKSPST GTTEISSHES SHGTPSQTTA
KNWELTASAS HQPPGVYPQG HSDITVAIST STVLLCGLSA VSLLACYLKS RQTPPLASVE
MEAMEALPVT WGTSSRDEDL ENCSHHL
Amino acid sequence of IL-15 receptor alpha Sushi domain
SEQ ID NO: 9
ITCPPPMSVE HADIWVKSYS LYSRERYICN SGFKRKAGTS SLTECVLNKA TNVAHWTTPS
LKCIR
Human CCL19 amino acid sequence
SEQ ID NO: 10
TNDAEDCC LSVTQKPIPG YIVRNFHYLL IKDGCRVPAV VFTTLRGRQL CAPPDQPWVE
RIIQRLQRTS AKMKRRSS
Peptide Linker (noncleavable)
SEQ ID NOs:11-16
(SEQ ID NO: 11)
GGGGS
(SEQ ID NO: 12)
GGGGSGGGGS
(SEQ ID NO: 13)
GGGGSGGGGS GGGGS
(SEQ ID NO: 14)
GGGGSGGGGS XGGGGSGGGG S,
X = A or N
(SEQ ID NO: 15)
GGGGSGGGGS XGGGGYGGGG S,
X = S, A or N, and Y = A or N
(SEQ ID NO: 16)
GGGGSGGGGS GGGGSAAGGG GSGGGGSGGG GS
MMP-2/MMP-9 cleavable peptide linkers
SEQ ID NOs: 17-23
(SEQ ID NO: 17)
GPLGVR
(SEQ ID NO: 18)
PLGMWSR
(SEQ ID NO: 19)
PLGLWAR
(SEQ ID NO: 20)
PQGIAGQR
(SEQ ID NO: 21)
PLGLAG
(SEQ ID NO: 22)
LALGPR
(SEQ ID NO: 23)
GGPLGMLSQS
Urokinase plasminogen activator (uPA) cleavable peptide linkers
SEQ ID NOs: 24-32
(SEQ ID NO: 24)
GGGGRRGGS
(SEQ ID NO: 25)
TGRGPSWV
(SEQ ID NO: 26)
SARGPSRW
(SEQ ID NO: 27)
TARGPSFK
(SEQ ID NO: 28)
TARGPSW
(SEQ ID NO: 29)
GGWHTGRN
(SEQ ID NO: 30)
HTGRSGAL
(SEQ ID NO: 31)
PLTGRSGG
(SEQ ID NO: 32)
LTGRSGA
matriptase cleavable peptide linker
SEQ ID NO: 33
RQARVVNG
matriptase-MMP2/9 dual cleavable peptide linker
SEQ ID NO: 34
VHMPLGFLGP RQARVVNG
cleavable peptide linker
SEQ ID NO: 35
GGSLSGRSDN HGGGGS
cleavable linker
SEQ ID NO: 36
GGGGSGGGGS GGGGSISSGL LSSGGSGGSL SGRSDNHGGG GS
Amino acid sequence of IgG1 Fc fused with IL-2Rβ; Fc
with hole mutations and LALA mutations (CX5.51.1)
SEQ ID NO: 37

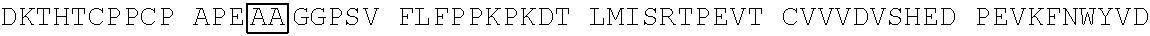

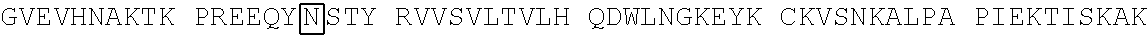

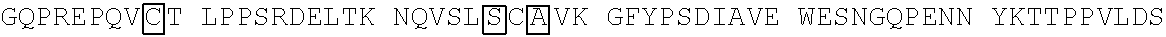

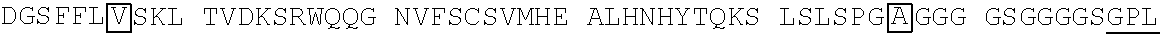

GVRGGGGSGG GGSAVNGTSQ FICEYNSRAN ISCVWSQDGA LQDTSCQVHA WPDRRRWNQT
CELLPVSQAS WACNLILGAP DSQKLTTVDI VTLRVLCREG VRWRVMAIQD FKPFENLRLM
APISLQVVHV ETHRCNISWE ISQASHYFER HLEFEARTLS PGHTWEEAPL LTLKQKQEWI
CLETLTPDTQ YEFQVRVKPL QGEFTTWSPW SQPLAFRTKP AALGKDT
Amino acid sequence of IgG1 Fc fused with IL-15Rα
Sushi then IL-15 polypeptide; Fc with knob mutations and LALA
mutations (CX5.51.4)
SEQ ID NO: 38

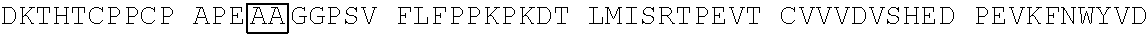

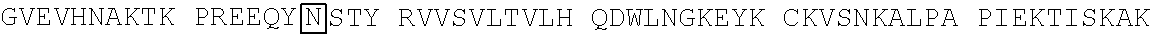

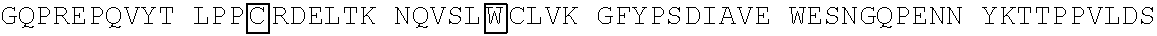

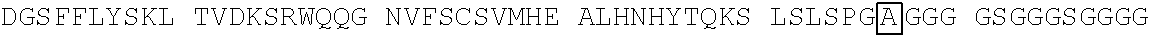

SITCPPPMSV EHADIWVKSY SLYSRERYIC NSGFKRKAGT SSLTECVLNK ATNVAHWTTP
SLKCIRDPAL VHQRPAPPSG GGGSGGGGSG GGGSNWVNVI SDLKKIEDLI QSMHIDATLY
TESDVHPSCK VTAMKCFLLE LQVISLESGD ASIHDTVEX₁LIILANNSLS SNGNVTESGC
KECEELEEKN IKEFLQSFVH IVX₂MFINTS;
wherein X₁ is an amino acid selected from N and D, and X₂is an amino
acid selected from Q and E.
Amino acid sequence of Fc fused with IL-15Rα Sushi
then IL-15 polypeptide; Fc with knob mutations; with long linker
between A and S
SEQ ID NO: 39

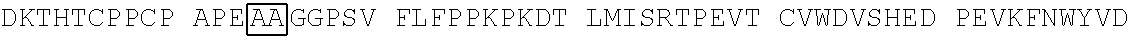

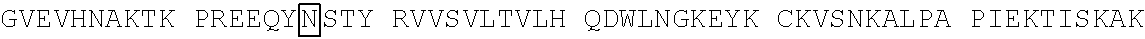

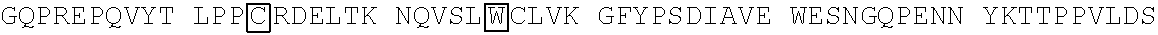

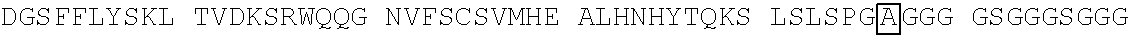

SITCPPPMSV EHADIWVKSY SLYSRERYIC NSGFKRKAGT SSLTECVLNK ATNVAHWTTP
SLKCIRDPAL VHQRPAPPSG GGGSGGGGSG GGGSAAGGGG SGGGGSGGGG SNWVNVISDL
KKIEDLIQSM HIDATLYTES DVHPSCKVTA MKCFLLELQV ISLESGDASI HDTVEX₁LII
LANNSLSSNG NVTESGCKEC EELEEKNIKE FLQSFVHIVX ₂MFINTS; wherein X₁ is an
amino acid selected from N and D, and X₂ is an amino acid selected from
Q and E.
Fc-IL-15-Sushi knob CX5.51.5
SEQ ID NO: 40

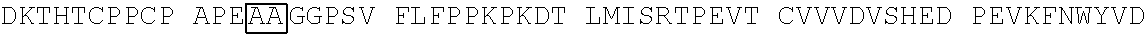

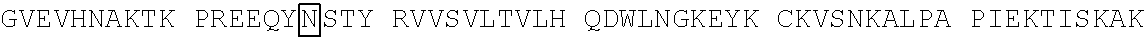

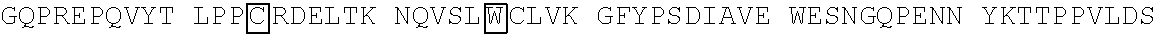

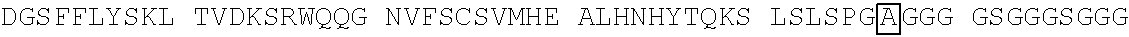

SNWVNVISDL KKIEDLIQSM HIDATLYTES DVHPSCKVTA MKCFLLELQV ISLESGDASI
HDTVENLIIL ANNSLSSNGN VTESGCKECE ELEEKNIKEF LQSFVHIVQM FINTSGGSGG
GGSGGGGSGG GGSITCPPPM SVEHADIWVK SYSLYSRERY ICNSGFKRKA GTSSLTECVL
NKATNVAHWT TPSLKCIRDP ALVHQRPAPP S**
Fc-IL-15 knob CX5.51.6
SEQ ID NO: 41

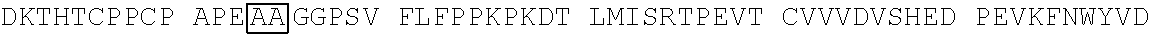

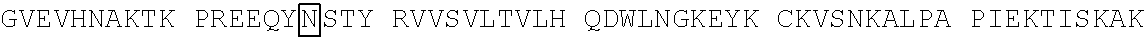

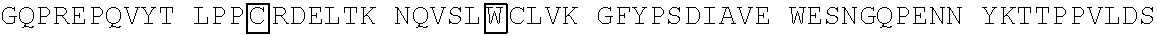

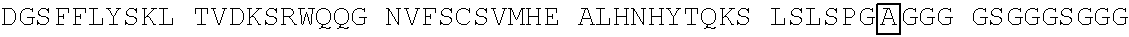

SNWVNVISDL KKIEDLIQSM HIDATLYTES DVHPSCKVTA MKCFLLELQV ISLESGDASI
HDTVENLIIL ANNSLSSNGN VTESGCKECE ELEEKNIKEF LQSFVHIVQM FINTS**
Fc-Sushi-beta hole (CX5.51.7)
SEQ ID NO: 42

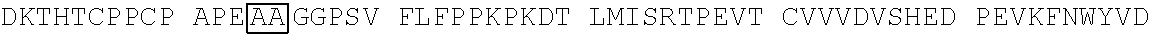

GVEVHNAKTK PREEQYNSTY RVVSVLTVLH QDWLNGKEYK CKVSNKALPA PIEKTISKAK

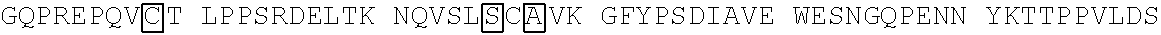

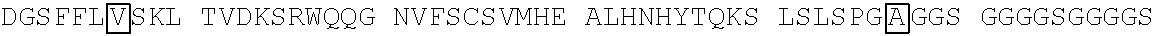

GGGGSITCPP PMSVEHADIW VKSYSLYSRE RYICNSGFKR KAGTSSLTEC VLNKATNVAH
WTTPSLKCIR DPALVHQRPA PPSGGGGSGG GGSGPLGVRG GGGSGGGGSA VNGTSQFTCF
YNSRANISCV WSQDGALQDT SCQVHAWPDR RRWNQTCELL PVSQASWACN LILGAPDSQK
LTTVDIVTLR VLCREGVRWR VMAIQDFKPF ENLRLMAPIS LQVVHVETHR CNISWEISQA
SHYFERHLEF EARTLSPGHT WEEAPLLTLK QKQEWICLET LTPDTQYEFQ VRVKPLQGEF
TTWSPWSQPL AFRTKPAALG KDT**
IgG1 Fc-Hole (CX5.43.8)
SEQ ID NO: 43
DKTHTCPPCP APELLGGPSV FLFPPKPKDT LMISRTPEVT CVVVDVSHED PEVKFNWYVD
GVEVHNAKTK PREEQYNSTY RVVSVLTVLH QDWLNGKEYK CKVSNKALPA PIEKTISKAK

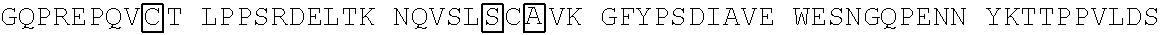

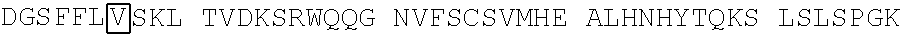

Fc-IL-15 knob, IL-15 mutein E46K/N65D
SEQ ID NO: 44

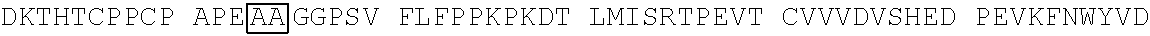

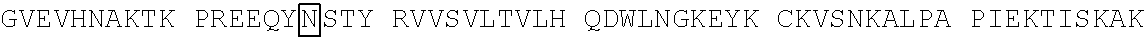

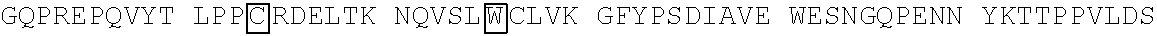

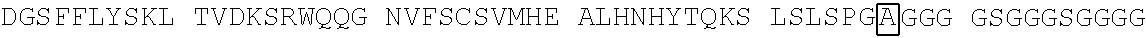

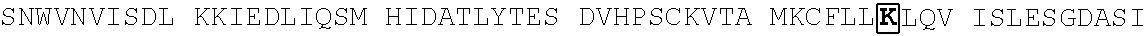

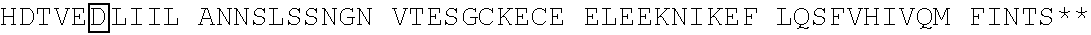

PD-Ll antibody 1296 heavy chain fused with Sushi and
then with IL-15 polypeptide, Fc with Knob mutations (CX5.48.1)
SEQ ID NO: 45

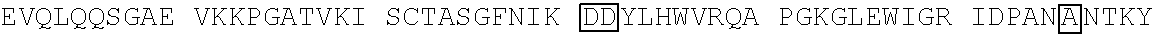

APKFQDRVTI TADTSTNTAY LELSSLRSED TAVYYCAARF GYFYGSSFYA VAYWGGILV
TVSSASTKGP SVFPLAPSSK STSGGTAALG CLVKDYFPEP VTVSWNSGAL TSGVHTFPAV
LQSSGLYSLS SVVTVPSSSL GTQTYICNVN HKPSNTKVDK KVEPKSCDKT HTCPPCPAPE
LLGGPSVFLF PPKPKDTLMI SRTPEVTCVV VDVSHEDPEV KFNWYVDGVE VHNAKTKPRE

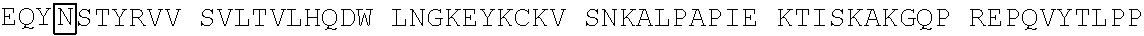

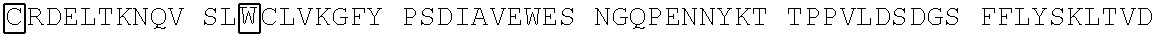

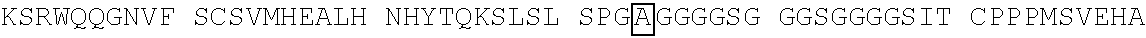

DIWVKSYSLY SRERYICNSG FKRKAGTSSL TECVLNKATN VAHWTTPSLK CIRDPALVHQ
RPAPPSGGGG SGGGGSGGGG SAAGGGGSGG GGSGGGGSNW VNVISDLKKI EDLIQSMHID
ATLYTESDVH PSCKVTAMKC FLLELQVISL ESGDASIHDT VEX₁LIILAN NSLSSNGNVT
ESGCKECEEL EEKNIKEFLQ SFVHIVX₂MF INTS; wherein X₁ is an amino acid
selected from N and D, and X₂ is an amino acid selected from Q and E.
PD-Ll antibody 1296 heavy chain-IL-15 then with the
Sushi domain, Fc with Knob mutations (CX5.48.2)
SEQ ID NO: 46

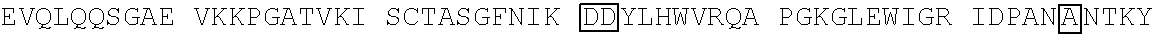

APKFQDRVTI TADTSTNTAY LELSSLRSED TAVYYCAARF GYFYGSSFYA VAYWGQGTLV
TVSSASTKGP SVFPLAPSSK STSGGTAALG CLVKDYFPEP VTVSWNSGAL TSGVHTFPAV
LQSSGLYSLS SVVTVPSSSL GTQTYICNVN HKPSNTKVDK KVEPKSCDKT HTCPPCPAPE
LLGGPSVFLF PPKPKDTLMI SRTPEVTCVV VDVSHEDPEV KFNWYVDGVE VHNAKTKPRE
EQYNSTYRVV SVLTVLHQDW LNGKEYKCKV SNKALPAPIE KTISKAKGQP REPQVYTLPP

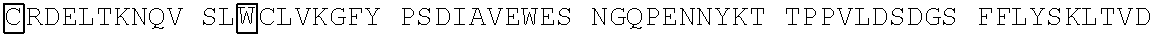

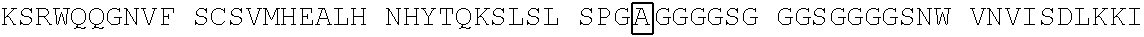

EDLIQSMHID ATLYTESDVH PSCKVTAMKC FLLELQVISL ESGDASIHDT VENLIILANN
SLSSNGNVTE SGCKECEELE EKNIKEFLQS FVHIVQMFIN TSGGSGGGGS GGGGSAAGGG
GSGGGGSGGG GSITCPPPMS VEHADIWVKS YSLYSRERYI CNSGFKRKAG TSSLTECVLN
KAINVAHWIT PSLKCIRDPA LVHQRPAPPS
PD-Ll antibody 1296 heavy chain fused with IL-2Rβ ECD,
Fc with Hole Mutations
SEQ ID NO: 47

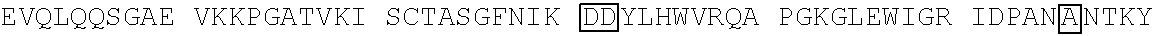

APKFQDRVTI TADTSTNTAY LELSSLRSED TAVYYCAARF GYFYGSSFYA VAYWGQGTLV
TVSSASTKGP SVFPLAPSSK STSGGTAALG CLVKDYFPEP VTVSWNSGAL TSGVHTFPAV
LQSSGLYSLS SVVTVPSSSL GTQTYICNVN HKPSNTKVDK KVEPKSCDKT HTCPPCPAPE
LLGGPSVFLF PPKPKDTLMI SRTPEVTCVV VDVSHEDPEV KFNWYVDGVE VHNAKTKPRE

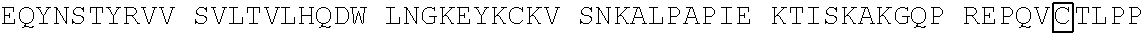

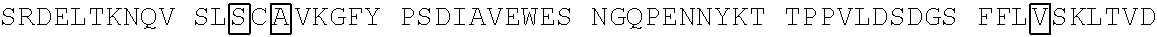

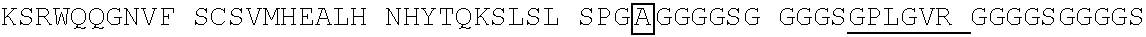

AVNGTSQFTC FYNSRANISC VWSQDGALQD TSCQVHAWPD RRRWNQTCEL LPVSQASWAC
NLILGAPDSQ KLTTVDIVTL RVLCREGVRW RVMAIQDFKP FENLRLMAPI SLQVVHVETH
RCNISWEISQ ASHYFERHLE FEARTLSPGH TWEEAPLLTL KQKQEWICLE TLTPDTQYEF
QVRVKPLQGE FTTWSPWSQP LAFRTKPAAL GKDT
PD-Ll antibody 1296 heavy chain fused with scFv1 (VH-VL)
which binds to IL-15, Fc with Hole Mutations
SEQ ID NO: 48

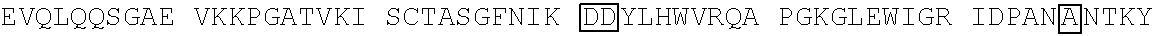

APKFQDRVTI TADTSTNTAY LELSSLRSED TAVYYCAARF GYFYGSSFYA VAYWGQGTLV
TVSSASTKGP SVFPLAPSSK STSGGTAALG CLVKDYFPEP VTVSWNSGAL TSGVHTFPAV
LQSSGLYSLS SVVTVPSSSL GTQTYICNVN HKPSNTKVDK KVEPKSCDKT HTCPPCPAPE
LLGGPSVFLF PPKPKDTLMI SRTPEVTCVV VDVSHEDPEV KFNWYVDGVE VHNAKTKPRE

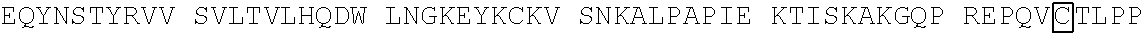

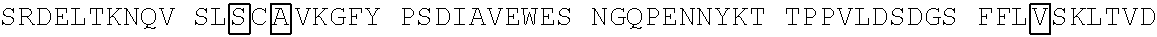

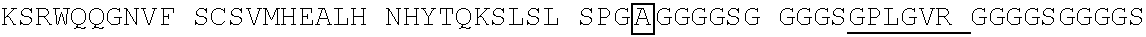

EVQLVQSGAE VKKPGESLKI SCKVSGYFFT TYWIGWVRQM PGKGLEYMGI IYPGDSDTRY
SPSFQGQVTI SADKSISTAY LQWSSLKASD TAMYYCARGG NWNCFDYWGQ GTLVTVSSGG
GGSGGGGSGG GGSEIVLTQS PGTLSLSPGR EATLSCRASQ SVSSSYLAWY QQKPGQAPRL
LIYGASRRAT GIPDRFSGSG SGTDFTLTIS RLEPEDFAVY YCQRYGSSHT FGQGTKLEIS R
PD-Ll antibody 1296 heavy chain fused with scFv2 (VL-VH)
which binds to IL-15, Fc with Hole Mutations
SEQ ID NO: 49

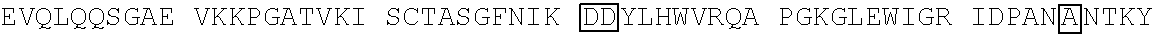

APKFQDRVTI TADTSTNTAY LELSSLRSED TAVYYCAARF GYFYGSSFYA VAYWGGILV
TVSSASTKGP SVFPLAPSSK STSGGTAALG CLVKDYFPEP VTVSWNSGAL TSGVHTFPAV
LQSSGLYSLS SVVTVPSSSL GTQTYICNVN HKPSNTKVDK KVEPKSCDKT HTCPPCPAPE
LLGGPSVFLF PPKPKDTLMI SRTPEVTCVV VDVSHEDPEV KFNWYVDGVE VHNAKTKPRE

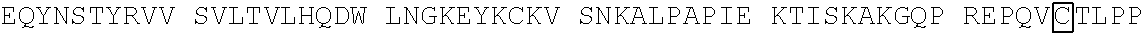

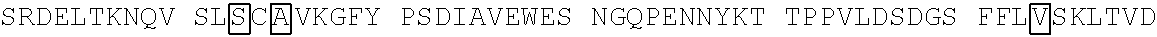

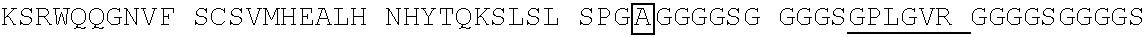

EIVLTQSPGT LSLSPGREAT LSCRASQSVS SSYLAWYQQK PGQAPRLLIY GASRRATGIP
DRFSGSGSGT DFTLTISRLE PEDFAVYYCQ RYGSSHTFGQ GTKLEISRGG GGSGGGGSGG
GGSEVQLVQS GAEVKKPGES LKISCKVSGY FFTTYWIGWV RQMPGKGLEY MGIIYPGDSD
TRYSPSFQGQ VTISADKSIS TAYLQWSSLK ASDTAMYYCA RGGNWNCFDY WGQGTLVTVS S
PD-Ll antibody 1296 LC
SEQ ID NO: 50
DIQMTQspSS LSASvGDRVT ItCRASQDIS NYLNWYQQKP DGTVKLLIYY TSRLHSGVPS
RFSGSGSGTD YtLTISsLqp EDIATYFCQQ GKTLPPTFGG GTKLEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
PD-Ll antibody 1296 LC fused with basal IL-2v
SEQ ID NO: 51
DIQMTQspSS LSASvGDRVT ItCRASQDIS NYLNWYQQKP DGTVKLLIYY TSRLHSGVPS
RFSGSGSGTD YtLTISsLqp EDIATYFCQQ GKTLPPTFGG GTKLEIKRTV AAPSVFIFPP
SDEQLKSGTA SVVCLLNNFY PREAKVQWKV DNALQSGNSQ ESVTEQDSKD STYSLSSTLT

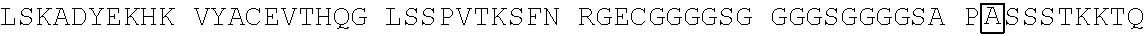

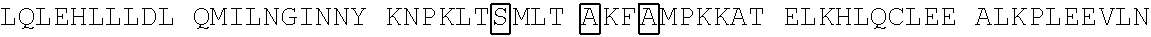

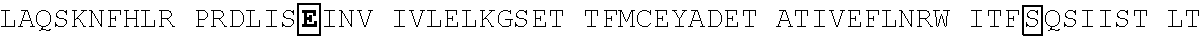

PD-1 antibody heavy chain fused with Sushi domain and
then with IL-15 polypeptide, Fc with Knob mutations (CX5.48.3)
SEQ ID NO: 52
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

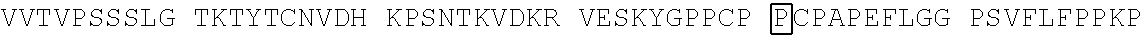

KDTLMISRTP EVTCVVVDVS QEDPEVQFNW YVDGVEVHNA KTKPREEQFN STYRVVSVLT

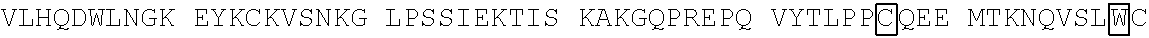

LVKGFYPSDI AVEWESNGQP ENNYKTTPPV LDSDGSFFLY SRLTVDKSRW QEGNVFSCSV

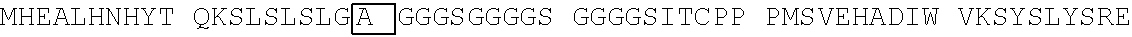

RYICNSGFKR KAGTSSLTEC VLNKATNVAH WTTPSLKCIR DPALVHQRPA PPSGGSGGGG
SGGGGSGGGG SNWVNVISDL KKIEDLIQSM HIDATLYTES DVHPSCKVTA MKCFLLELQV
ISLESGDASI HDTVENLIIL ANNSLSSNGN VTESGCKECE ELEEKNIKEF LQSFVHIVQM
FINIS
PD-1 antibody heavy chain-IL-15 then with the Sushi
domain, Fc with Knob mutations (CX5.48.4)
SEQ ID NO: 53
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

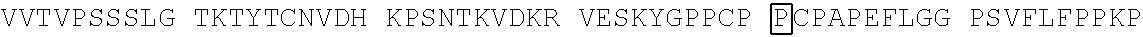

KDTLMISRTP EVTCVVVDVS QEDPEVQFNW YVDGVEVHNA KTKPREEQFN STYRVVSVLT

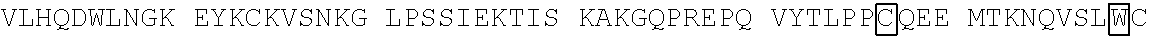

LVKGFYPSDI AVEWESNGQP ENNYKTTPPV LDSDGSFFLY SRLTVDKSRW QEGNVFSCSV

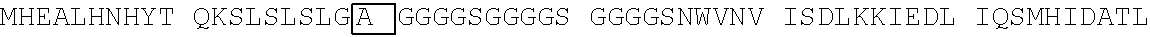

YTESDVHPSC KVTAMKCFLL ELQVISLESG DASIHDTVEN LIILANNSLS SNGNVTESGC
KECEELEEKN IKEFLQSFVH IVQMFINTSG GSGGGGSGGG GSGGGGSITC PPPMSVEHAD
IWVKSYSLYS RERYICNSGF KRKAGTSSLT ECVLNKATNV AHWTTPSLKC IRDPALVHQR
PAPPS
PD-1 antibody HC-beta hole (CX3.58.3)
SEQ ID NO: 54
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

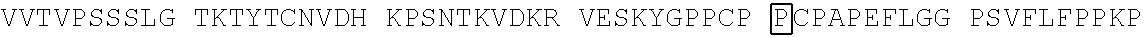

KDTLMISRTP EVTCVVVDVS QEDPEVQFNW YVDGVEVHNA KTKPREEQFN STYRVVSVLT

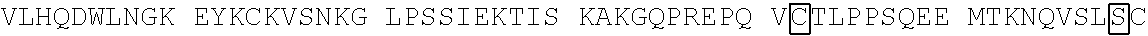

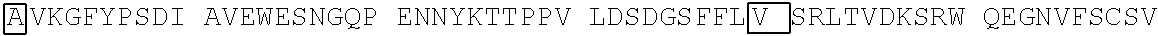

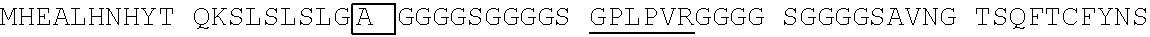

RANISCVWSQ DGALQDTSCQ VHAWPDRRRW NQTCELLPVS QASWACNLIL GAPDSQKLTT
VDIVTLRVLC REGVRWRVMA IQDFKPFENL RLMAPISLQV VHVETHRCNI SWEISQASHY
FERHLEFEAR TLSPGHTWEE APLLTLKQKQ EWICLETLTP DTQYEFQVRV KPLQGEFTTW
SPWSQPLAFR TKPAALGKDT
PD-1 antibody LC (CX5.17.1)
SEQ ID NO: 55
EIVLTQSPAT LSLSPGERAT LSCRASQSVS SYLAWYQQKP GQAPRLLIYD ASNRATGIPA
RFSGSGSGTD FTLTISSLEP EDFAVYYCQQ SSNWPRTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
PD-1 antibody LC fused with basal IL-2v
SEQ ID NO: 56
EIVLTQSPAT LSLSPGERAT LSCRASQSVS SYLAWYQQKP GQAPRLLIYD ASNRATGIPA
RFSGSGSGTD FTLTISSLEP EDFAVYYCQQ SSNWPRTFGQ GTKVEIKRTV AAPSVFIFPP
SDEQLKSGTA SVVCLLNNFY PREAKVQWKV DNALQSGNSQ ESVTEQDSKD STYSLSSTLT

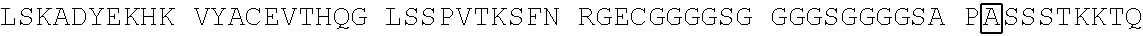

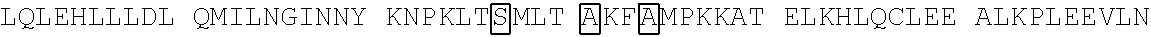

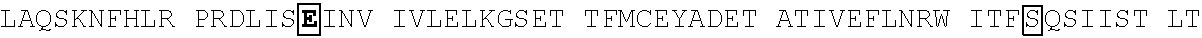

PD-1 HC hole (CX3.58.4)
SEQ ID NO: 57
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

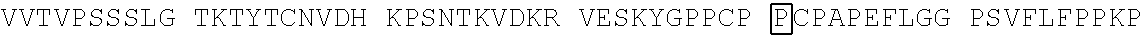

KDTLMISRTP EVTCVVVDVS QEDPEVQFNW YVDGVEVHNA KTKPREEQFN STYRVVSVLT

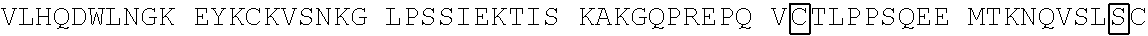

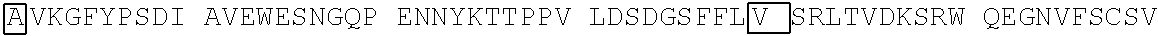

MHEALHNHYT QKSLSLSLGK
PD-1 antibody heavy chain fused with Sushi domain and
then with IL-15 polypeptide N65D, Fc with Knob mutations
SEQ ID NO: 58
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

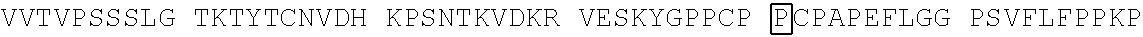

KDTLMISRTP EVTCVVVDVS QEDPEVQFNW YVDGVEVHNA KTKPREEQFN STYRVVSVLT

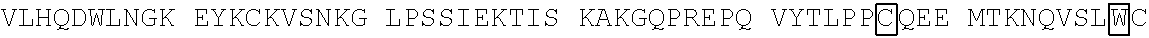

LVKGFYPSDI AVEWESNGQP ENNYKTTPPV LDSDGSFFLY SRLTVDKSRW QEGNVFSCSV

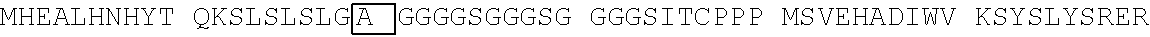

YICNSGFKRK AGTSSLTECV LNKATNVAHW TTPSLKCIRD PALVHQRPAP PSGGGGSGGG
GSGGGGSNWV NVISDLKKIE DLIQSMHIDA TLYTESDVHP SCKVTAMKCF LLELQVISLE

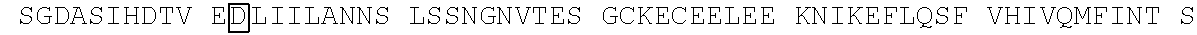

PD-1 antibody heavy chain fused with Sushi domain and
then with IL-15 polypeptide Q108E, Fc with Knob mutations
SEQ ID NO: 59
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

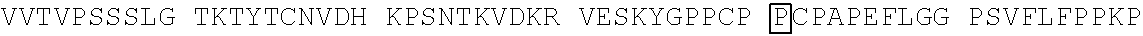

KDTLMISRTP EVTCVVVDVS QEDPEVQFNW YVDGVEVHNA KTKPREEQFN STYRVVSVLT

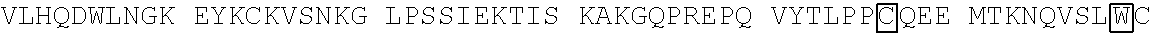

LVKGFYPSDI AVEWESNGQP ENNYKTTPPV LDSDGSFFLY SRLTVDKSRW QEGNVFSCSV

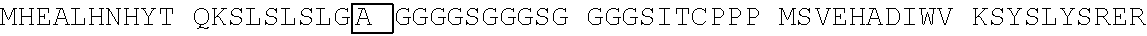

YICNSGFKRK AGTSSLTECV LNKATNVAHW TTPSLKCIRD PALVHQRPAP PSGGGGSGGG
GSGGGGSNWV NVISDLKKIE DLIQSMHIDA TLYTESDVHP SCKVTAMKCF LLELQVISLE

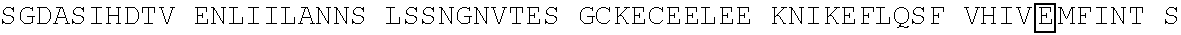

PD-1 antibody HC-scFV1 (VH-VL) hole
SEQ ID NO: 60
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

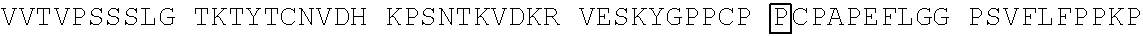

KDTLMISRTP EVTCVVVDVS QEDPEVQFNW YVDGVEVHNA KTKPREEQFN STYRVVSVLT

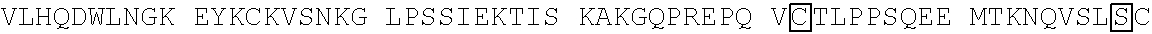

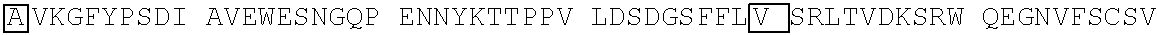

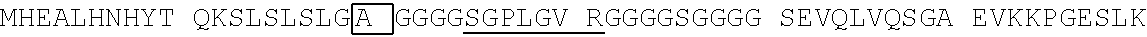

ISCKVSGYFF TTYWIGWVRQ MPGKGLEYMG IIYPGDSDTR YSPSFQGQVT ISADKSISTA
YLQWSSLKAS DTAMYYCARG GNWNCFDYWG QGTLVTVSSG GGGSGGGGSG GGGSEIVLTQ
SPGTLSLSPG REATLSCRAS QSVSSSYLAW YQQKPGQAPR LLIYGASRRA TGIPDRFSGS
GSGTDFTLTI SRLEPEDFAV YYCQRYGSSH TFGQGTKLEI SR
PD-1 antibody HC-scfv2 (VL-VH) hole
SEQ ID NO: 61
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

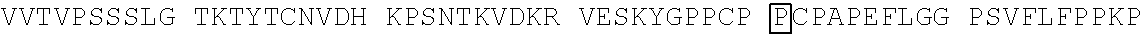

KDTLMISRTP EVTCVVVDVS QEDPEVQFNW YVDGVEVHNA KTKPREEQFN STYRVVSVLT

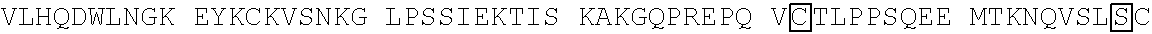

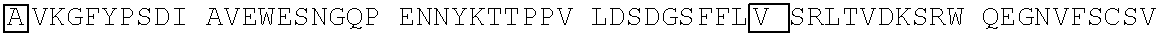

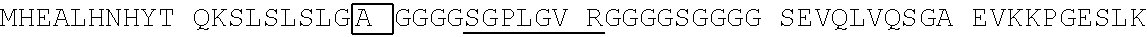

TLSCRASQSV SSSYLAWYQQ KPGQAPRLLI YGASRRATGI PDRFSGSGSG TDFTLTISRL
EPEDFAVYYC QRYGSSHTFG QGTKLEISRG GGGSGGGGSG GGGSEVQLVQ SGAEVKKPGE
SLKISCKVSG YFFTTYWIGW VRQMPGKGLE YMGIIYPGDS DTRYSPSFQG QVTISADKSI
STAYLQWSSL KASDTAMYYC ARGGNWNCFD YWGQGTLVTV SS
PD-1 antibody heavy chain fused with IL-15 polypeptide
E46K, Fc with Knob mutations; no Sushi
SEQ ID NO: 62
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

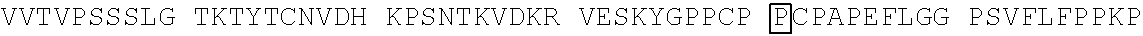

KDTLMISRTP EVTCVVVDVS QEDPEVQFNW YVDGVEVHNA KTKPREEQFN STYRVVSVLT

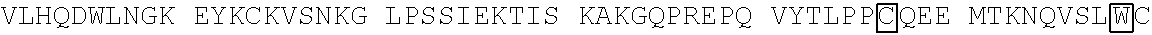

LVKGFYPSDI AVEWESNGQP ENNYKTTPPV LDSDGSFFLY SRLTVDKSRW QEGNVFSCSV

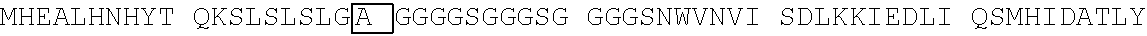

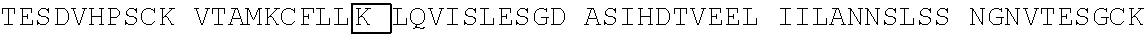

ECEELEEKNI KEFLQSFVHI VQMFINTS
PD-1 antibody heavy chain fused with IL-15 polypeptide
E46K/N65D, Fc with Knob mutations; no Sushi
SEQ ID NO: 63
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

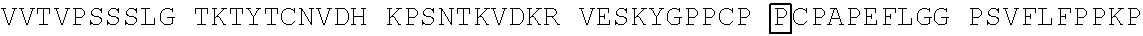

KDTLMISRTP EVTCVVVDVS QEDPEVQFNW YVDGVEVHNA KTKPREEQFN STYRVVSVLT

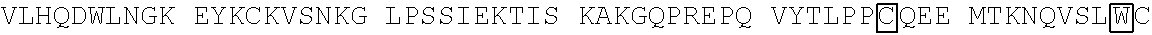

LVKGFYPSDI AVEWESNGQP ENNYKTTPPV LDSDGSFFLY SRLTVDKSRW QEGNVFSCSV

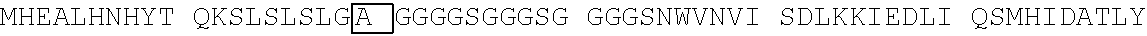

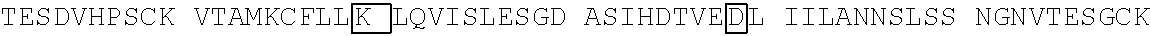

ECEELEEKNI KEFLQSFVHI VQMFINTS
CX7_71_1 PD1-IL-15vE46K, no Sushi, no KIH
SEQ ID NO: 64
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

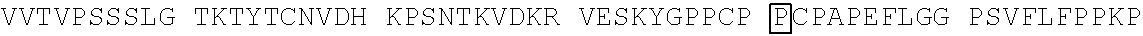

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

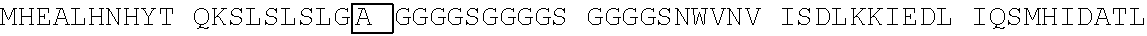

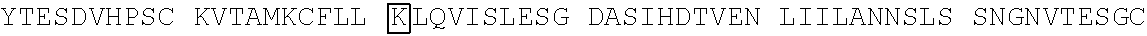

KECEELEEKN IKEFLQSFVH IVQMFINTS
PD1-IL-15vE46K/N65D, no Sushi, no KIH
SEQ ID NO: 65
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

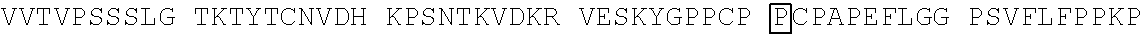

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

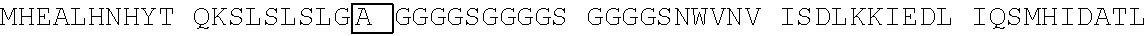

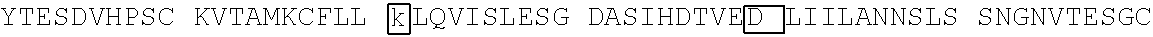

KECEELEEKN IKEFLQSFVH IVQMFINTS
CX7_53_2 PD1-ScFv2 no KIH
SEQ ID NO: 66
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

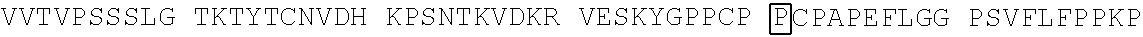

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

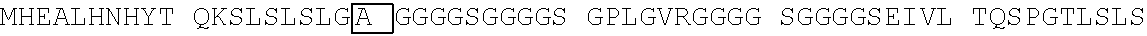

PGERATLSCR ASQSVSSSYL AWYQQKPGQA PRLLIYGASR RATGIPDRFS GSGSGTDFTL
TISRLEPEDF AVYYCQRYGS SHTFGQGTKL EISGGGGSGG GGSGGGGSEV QLVQSGAEVK
KPGESLKISC KVSGYFFTTY WIGWVRQMPG KGLEYMGIIY PGDSDTRYSP SFQGQVTISA
DKSISTAYLQ WSSLKASDTA MYYCARGGNW NCFDYWGQGT LVTVSS**
CX7_53_1 PD1-Sushi-IL-15vN65D no KIH
SEQ ID NO: 67
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

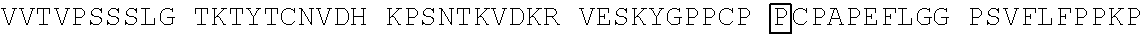

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

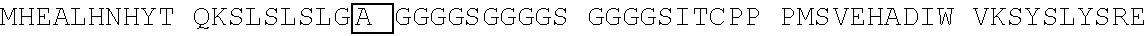

RYICNSGFKR KAGTSSLTEC VLNKATNVAH WTTPSLKCIR GGSGGGGSGG GSGGGGSNWV
NVISDLKKIE DLIQSMHIDA TLYTESDVHP SCKVTAMKCF LLELQVISLE SGDASIHDTV

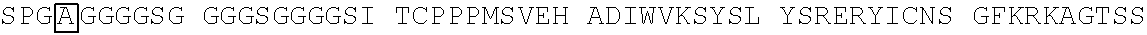

CX7_53_1 PD1-Sushi-IL-15vN65D no KIH, long linker
SEQ ID NO: 68
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

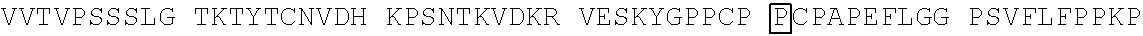

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

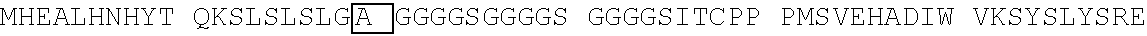

RYICNSGFKR KAGTSSLTEC VLNKATNVAH WTTPSLKCIR GGSGGGGSGG GSGGGGSAAG
GGGSGGGGSG GGGSNWVNVI SDLKKIEDLI QSMHIDATLY TESDVHPSCK VTAMKCFLLE

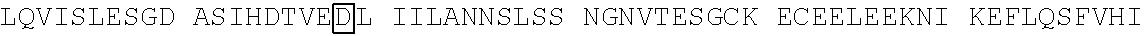

VQMFINTS
PD-1 antibody heavy chain fused with Sushi domain and
then with IL-15 polypeptide N65D, Fc with Knob mutations, long linker
SEQ ID NO: 69
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

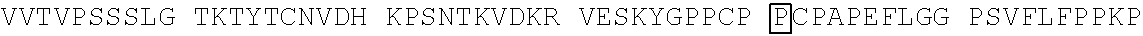

KDTLMISRTP EVTCVVVDVS QEDPEVQFNW YVDGVEVHNA KTKPREEQFN STYRVVSVLT

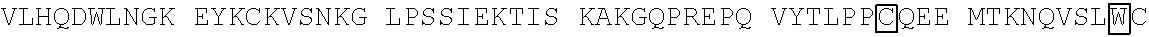

LVKGFYPSDI AVEWESNGQP ENNYKTTPPV LDSDGSFFLY SRLTVDKSRW QEGNVFSCSV

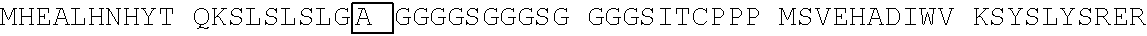

YICNSGFKRK AGTSSLTECV LNKATNVAHW TTPSLKCIRD PALVHQRPAP PSGGGGSGGG
GSGGGGSAAG GGGSGGGGSG GGGSNWVNVI SDLKKIEDLI QSMHIDATLY TESDVHPSCK
VTAMKCFLLE LQVISLESGD ASIHDTVEDL IILANNSLSS NGNVTESGCK ECEELEEKNI
KEFLQSFVHI VQMFINTS
Amino acid sequence of IgG1 Fc fused with scFv1 against
IL-15; Fc with hole mutations
SEQ ID NO: 70

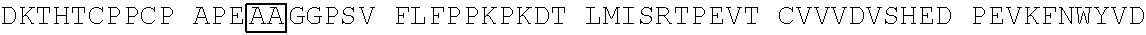

GVEVHNAKTK PREEQYNSTY RVVSVLTVLH QDWLNGKEYK CKVSNKALPA PIEKTISKAK

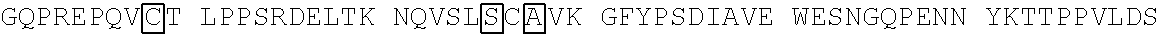

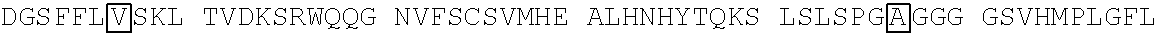

GPRQARVVNG GGGGSGGGGS EVQLVQSGAE VKKPGESLKI SCKVSGYFFT TYWIGWVRQM
PGKGLEYMGI IYPGDSDTRY SPSFQGQVTI SADKSISTAY LQWSSLKASD TAMYYCARGG
NWNCFDYWGQ GTLVTVSSGG GGSGGGGSGG GGSEIVLTQS PGTLSLSPGR EATLSCRASQ
SVSSSYLAWY QQKPGQAPRL LIYGASRRAT GIPDRFSGSG SGTDFTLTIS RLEPEDFAVY
YCQRYGSSHT FGQGTKLEIS R
Amino acid sequence of IgG1 Fc fused with scFv against
IL-15, ver2; Fc with hole mutations
SEQ ID NO: 71

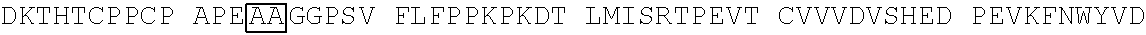

GVEVHNAKTK PREEQYNSTY RVVSVLTVLH QDWLNGKEYK CKVSNKALPA PIEKTISKAK

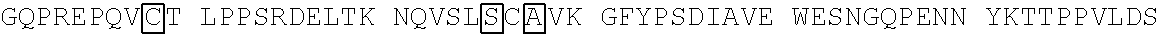

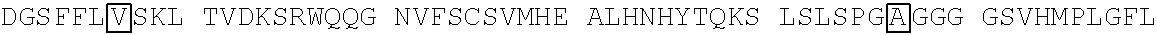

GPRQARVVNG GGGGSGGGGS EIVLTQSPGT LSLSPGREAT LSCRASQSVS SSYLAWYQQK
PGQAPRLLIY GASRRATGIP DRFSGSGSGT DFTLTISRLE PEDFAVYYCQ RYGSSHTFGQ
GTKLEISRGG GGSGGGGSGG GGSEVQLVQS GAEVKKPGES LKISCKVSGY FFTTYWIGWV
RQMPGKGLEY MGIIYPGDSD TRYSPSFQGQ VTISADKSIS TAYLQWSSLK ASDTAMYYCA
RGGNWNCFDY WGQGTLVTVS S
Amino acid sequence of Fc fused with scFv against IL-15,
ver3; Fc with hole mutations
SEQ ID NO: 72

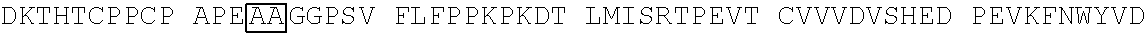

GVEVHNAKTK PREEQYNSTY RVVSVLTVLH QDWLNGKEYK CKVSNKALPA PIEKTISKAK

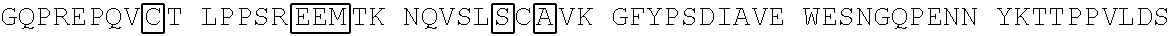

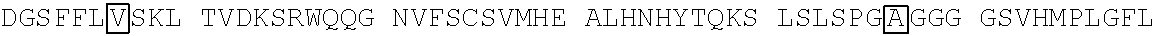

GPRQARVVNG GGGGSGGGGS EVQLVQSGAE VKKPGESLKI SCKVSGYFFT TYWIGWVRQM
PGKGLEYMGI IYPGDSDTRY SPSFQGQVTI SADKSISTAY LQWSSLKASD TAMYYCARGG
NWNCFDYWGQ GTLVTVSSGG GGSGGGGSGG GGSEIVLTQS PGTLSLSPGR EATLSCRASQ
SVSSSYLAWY QQKPGQAPRL LIYGASRRAT GIPDRFSGSG SGTDFTLTIS RLEPEDFAVY
YCQRYGSSHT FGQGTKLEIS R
Amino acid sequence of Fc fused with scFv against IL-15,
ver4; Fc with hole mutations
SEQ ID NO: 73

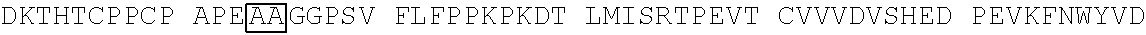

GVEVHNAKTK PREEQYNSTY RVVSVLTVLH QDWLNGKEYK CKVSNKALPA PIEKTISKAK

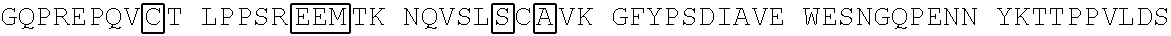

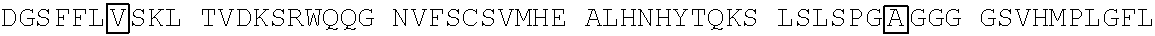

GPRQARVVNG GGGGSGGGGS EIVLTQSPGT LSLSPGREAT LSCRASQSVS SSYLAWYQQK
PGQAPRLLIY GASRRATGIP DRFSGSGSGT DFTLTISRLE PEDFAVYYCQ RYGSSHTFGQ
GTKLEISRGG GGSGGGGSGG GGSEVQLVQS GAEVKKPGES LKISCKVSGY FFTTYWIGWV
RQMPGKGLEY MGIIYPGDSD TRYSPSFQGQ VTISADKSIS TAYLQWSSLK ASDTAMYYCA
RGGNWNCFDY WGQGTLVTVS S
IgG1 Fc-knob-Sushi-IL-15 IgG1_allotype EEM, LALA
mutation H435RY436F
SEQ ID NO: 74

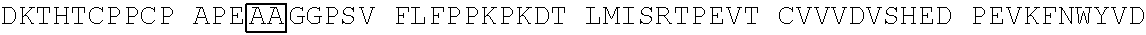

GVEVHNAKTK PREEQYNSTY RVVSVLTVLH QDWLNGKEYK CKVSNKALPA PIEKTISKAK

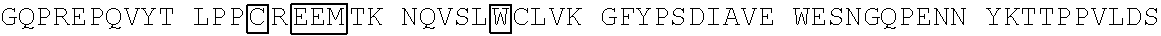

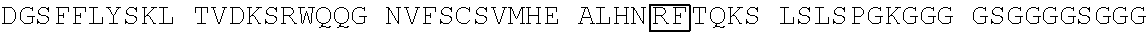

GSITCPPPMS VEHADIWVKS YSLYSRERYI CNSGFKRKAG TSSLTECVLN KAINVAHWIT
PSLKCIRGGS GGGGSGGGSG GGGSNWVNVI SDLKKIEDLI QSMHIDATLY TESDVHPSCK
VTAMKCFLLE LQVISLESGD ASIHDTVENL IILANNSLSS NGNVTESGCK ECEELEEKNI
KEFLQSFVHI VQMFINTS**
IgG1 Fc-knob-IL-15 (E46K/N65D) IgG1_allotype EEM, LALA mutation
SEQ ID NO: 75

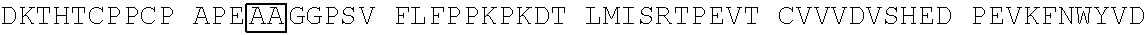

GVEVHNAKTK PREEQYNSTY RVVSVLTVLH QDWLNGKEYK CKVSNKALPA PIEKTISKAK

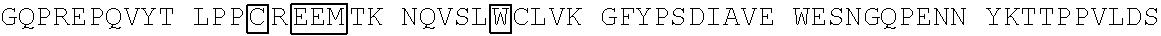

DGSFFLYSKL TVDKSRWQQG NVFSCSVMHE ALHNHYTQKS LSLSPGKGGG GSGGGGSGGG
GSITCPPPMS VEHADIWVKS YSLYSRERYI CNSGFKRKAG TSSLTECVLN KAINVAHWIT
PSLKCIRGGS GGGGSGGGSG GGGSNWVNVI SDLKKIEDLI QSMHIDATLY TESDVHPSCK

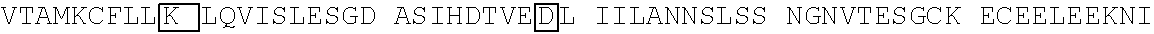

KEFLQSFVHI VQMFINTS**
IgG1 Fc-knob-Sushi-IL-15 (N65D) IgG1_allotype EEM, LALA
mutation H435R/Y436F
SEQ ID NO: 76

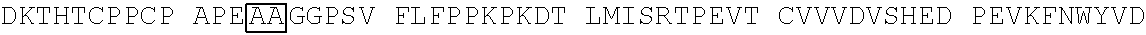

GVEVHNAKTK PREEQYNSTY RVVSVLTVLH QDWLNGKEYK CKVSNKALPA PIEKTISKAK

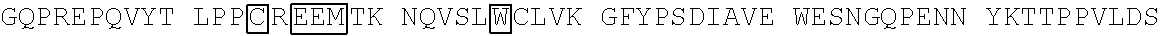

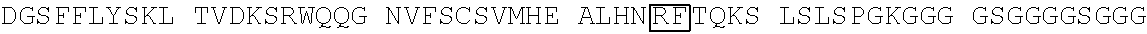

GSITCPPPMS VEHADIWVKS YSLYSRERYI CNSGFKRKAG TSSLTECVLN KAINVAHWIT
PSLKCIRGGS GGGGSGGGSG GGGSNWVNVI SDLKKIEDLI QSMHIDATLY TESDVHPSCK
VTAMKCFLLE LQVISLESGD ASIHDTVEDL IILANNSLSS NGNVTESGCK ECEELEEKNI
KEFLQSFVHI VQMFINTS**
IgG1 Fc-knob-Sushi-IL-15 IgG1_allotype EEM, LALA
mutation H435RY436F
5′XbaI,3′PmeI
ASKGD111_CX5_75_3
SEQ ID NO: 77

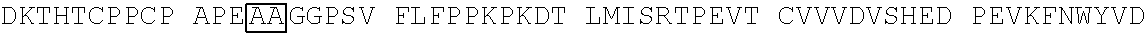

GVEVHNAKTK PREEQYNSTY RVVSVLTVLH QDWLNGKEYK CKVSNKALPA PIEKTISKAK

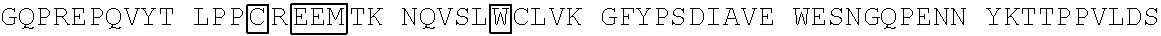

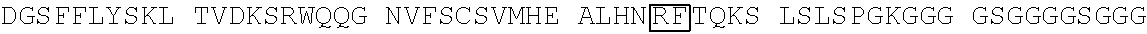

GSITCPPPMS VEHADIWVKS YSLYSRERYI CNSGFKRKAG TSSLTECVLN KAINVAHWIT
PSLKCIRGGS GGGGSGGGSG GGGSNWVNVI SDLKKIEDLI QSMHIDATLY TESDVHPSCK
VTAMKCFLLE LQVISLESGD ASIHDTVENL IILANNSLSS NGNVTESGCK ECEELEEKNI
KEFLQSFVHI VQMFINTS**
IgG1 Fc-knob-Sushi-IL-15 (D3ON,E64Q,N65D) IgG1_allotype
EEM, LALA mutation H435R/Y436F
5′XbaI,3′PmeI
ASKGD111_CX5_74_1
SEQ ID NO: 78

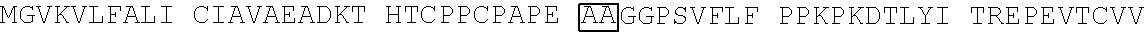

VDVSHEDPEV KFNWYVDGVE VHNAKTKPRE EQYNSTYRVV SVLTVLHQDW LNGKEYKCKV

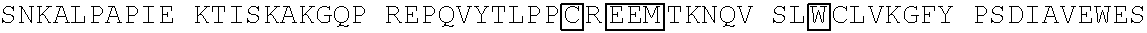

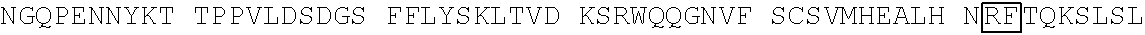

SPGKGGGGSG GGGSGGGGSI TCPPPMSVEH ADIWVKSYSL YSRERYICNS GFKRKAGISS
LTECVLNKAT NVAHWTTPSL KCIRGGSGGG GSGGGSGGGG SNWVNVISDL KKIEDLIQSM

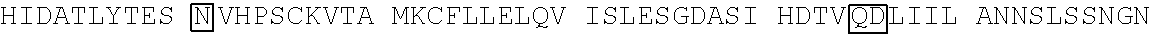

VTESGCKECE ELEEKNIKEF LQSFVHIVQM FINIS**
IgG1 Fc-knob-Sushi-IL-15 (N65D) IgG1_allotype EEM, LALA
mutation H435R/Y436F
5′XbaI,3′PmeI
ASKGD111_CX5_74_2
SEQ ID NO: 79

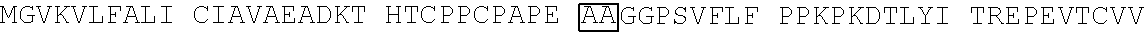

VDVSHEDPEV KFNWYVDGVE VHNAKTKPRE EQYNSTYRVV SVLTVLHQDW LNGKEYKCKV

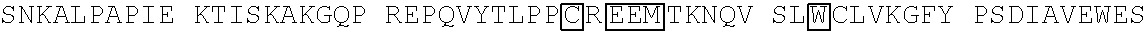

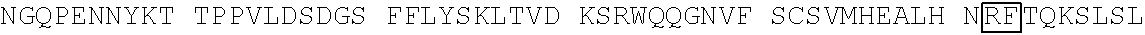

SPGKGGGGSG GGGSGGGGSI TCPPPMSVEH ADIWVKSYSL YSRERYICNS GFKRKAGISS
LTECVLNKAT NVAHWTTPSL KCIRGGSGGG GSGGGSGGGG SNWVNVISDL KKIEDLIQSM

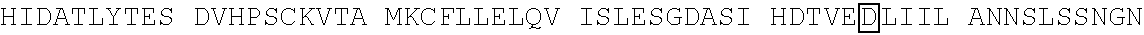

VTESGCKECE ELEEKNIKEF LQSFVHIVQM FINIS**
IgG4 FC-scFV1 (VH-VL) hole
SEQ ID NO: 80

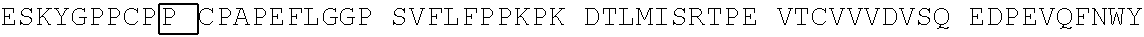

VDGVEVHNAK TKPREEQFNS TYRVVSVLTV LHQDWLNGKE YKCKVSNKGL PSSIEKTISK

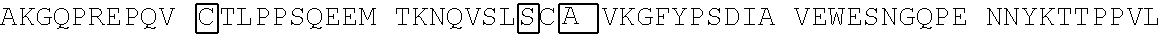

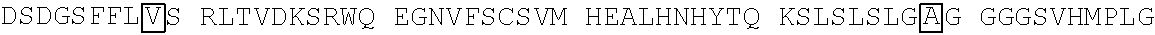

FLGPRQARVV NGGGGGSGGG GSEVQLVQSG AEVKKPGESL KISCKVSGYF FTTYWIGWVR
QMPGKGLEYM GIIYPGDSDT RYSPSFQGQV TISADKSIST AYLQWSSLKA SDTAMYYCAR
GGNWNCFDYW GQGTLVTVSS GGGGSGGGGS GGGGSEIVLT QSPGTLSLSP GREATLSCRA
SQSVSSSYLA WYQQKPGQAP RLLIYGASRR ATGIPDRFSG SGSGTDFTLT ISRLEPEDFA
VYYCQRYGSS HTFGQGTKLE ISR
IgG4 FC-scfv2 (VL-VH) hole
SEQ ID NO: 81

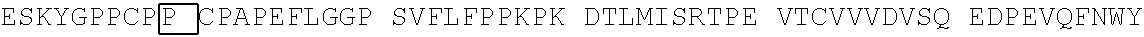

VDGVEVHNAK TKPREEQFNS TYRVVSVLTV LHQDWLNGKE YKCKVSNKGL PSSIEKTISK

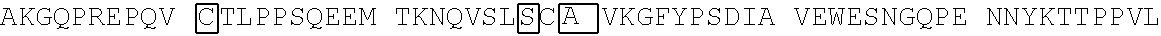

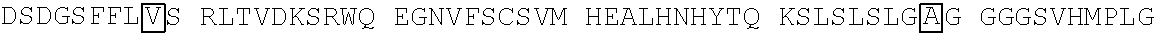

FLGPRQARVV NGGGGGSGGG GSEIVLTQSP GTLSLSPGRE ATLSCRASQS VSSSYLAWYQ
QKPGQAPRLL IYGASRRATG IPDRFSGSGS GTDFTLTISR LEPEDFAVYY CQRYGSSHTF
GQGTKLEISR GGGGSGGGGS GGGGSEVQLV QSGAEVKKPG ESLKISCKVS GYFFTTYWIG
WVRQMPGKGL EYMGIIYPGD SDTRYSPSFQ GQVTISADKS ISTAYLQWSS LKASDTAMYY
CARGGNWNCF DYWGQGTLVT VSS
IgG4 Fc-knob-Sushi-IL-15 (N65D)
SEQ ID NO: 82

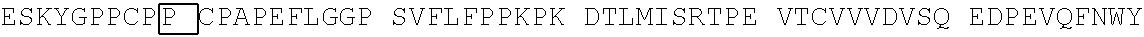

VDGVEVHNAK TKPREEQFNS TYRVVSVLTV LHQDWLNGKE YKCKVSNKGL PSSIEKTISK

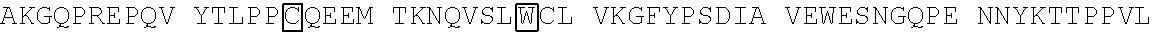

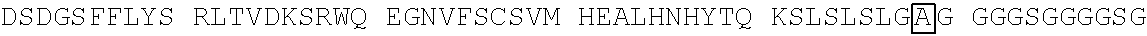

GGGSITCPPP MSVEHADIWV KSYSLYSRER YICNSGFKRK AGTSSLTECV LNKATNVAHW
TTPSLKCIRG GSGGGGSGGG SGGGGSNWVN VISDLKKIED LIQSMHIDAT LYTESDVHPS

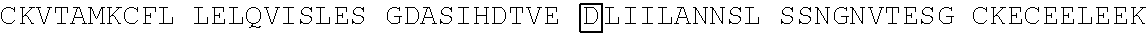

NIKEFLQSFV HIVQMFINTS
IgG4 Fc-knob-Sushi-IL-15 (N65D), long linker
SEQ ID NO: 83

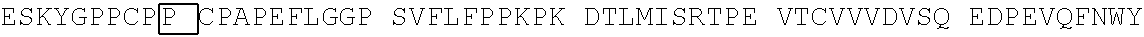

VDGVEVHNAK TKPREEQFNS TYRVVSVLTV LHQDWLNGKE YKCKVSNKGL PSSIEKTISK

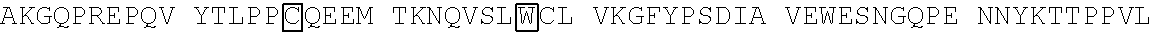

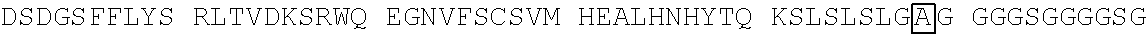

GGGSITCPPP MSVEHADIWV KSYSLYSRER YICNSGFKRK AGTSSLTECV LNKATNVAHW
TTPSLKCIRG GSGGGGSGGG SGGGGSAAGG SGGGGSGGGS GGGGSNWVNV ISDLKKIEDL

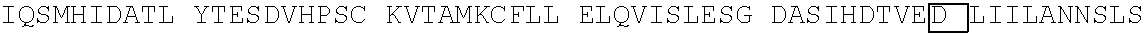

SNGNVTESGC KECEELEEKN IKEFLQSFVH IVQMFINTS
IgG4 Fc-knob-Sushi-IL-15 (Q108E)
SEQ ID NO: 84

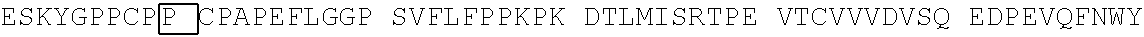

VDGVEVHNAK TKPREEQFNS TYRVVSVLTV LHQDWLNGKE YKCKVSNKGL PSSIEKTISK

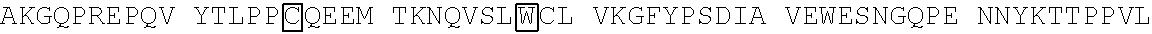

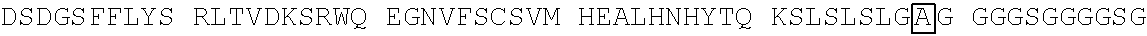

GGGSITCPPP MSVEHADIWV KSYSLYSRER YICNSGFKRK AGTSSLTECV LNKATNVAHW
TTPSLKCIRG GSGGGGSGGG SGGGGSNWVN VISDLKKIED LIQSMHIDAT LYTESDVHPS
CKVTAMKCFL LELQVISLES GDASIHDTVE NLIILANNSL SSNGNVTESG CKECEELEEK

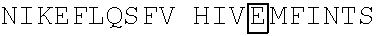

IgG4 Fc-knob-IL-15 E46K, no Sushi
SEQ ID NO: 85

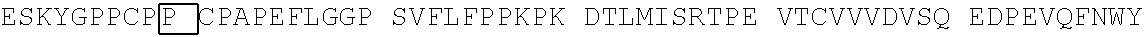

VDGVEVHNAK TKPREEQFNS TYRVVSVLTV LHQDWLNGKE YKCKVSNKGL PSSIEKTISK

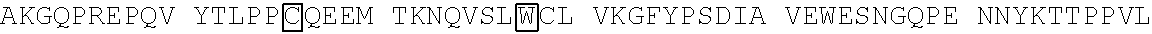

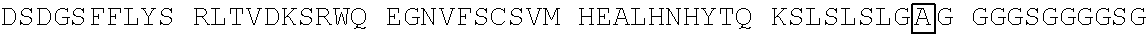

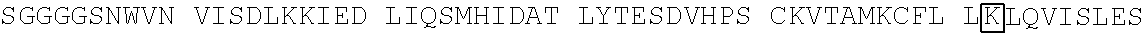

GDASIHDTVE NLIILANNSL SSNGNVTESG CKECEELEEK NIKEFLQSFV HIVQMFINTS
IgG4 Fc-knob-IL-15 E46K/N65D, no Sushi
SEQ ID NO: 86

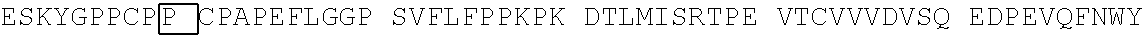

VDGVEVHNAK TKPREEQFNS TYRVVSVLTV LHQDWLNGKE YKCKVSNKGL PSSIEKTISK

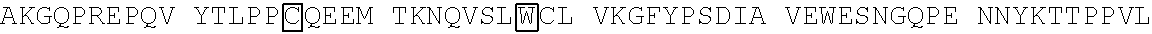

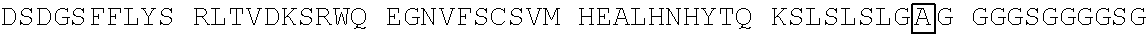

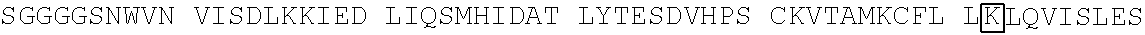

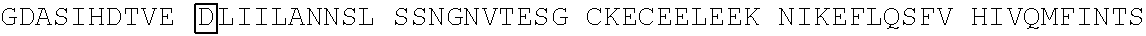

IgG4 FC-IL2R_beta ECD hole
SEQ ID NO: 87

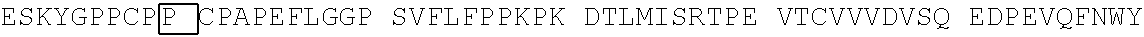

VDGVEVHNAK TKPREEQFNS TYRVVSVLTV LHQDWLNGKE YKCKVSNKGL PSSIEKTISK

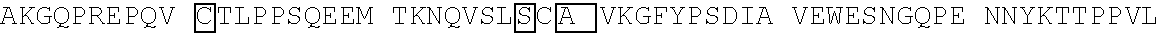

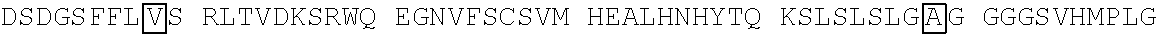

FLGPRQARVV NGGGGGSGGG GSGGGGSAVN GTSQFTCFYN SRANISCVWS QDGALQDTSC
QVHAWPDRRR WNQTCELLPV SQASWACNLI LGAPDSQKLT TVDIVTLRVL CREGVRWRVM
AIQDFKPFEN LRLMAPISLQ VVHVETHRCN ISWEISQASH YFERHLEFEA RTLSPGHTWE
EAPLLTLKQK QEWICLETLT PDTQYEFQVR VKPLQGEFTT WSPWSQPLAF RTKPAALGKD T
cetuximab light chain
SEQ ID NO: 88
DILLTQSPVI LSVSPGERVS FSCRASQSIG TNIHWYQQRT NGSPRLLIKY ASESISGIPS
RFSGSGSGTD FTLSINSVES EDIADYYCQQ NNNWPTTFGA GTKLELKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
cetuximab heavy chain
SEQ ID NO: 89
QVQLKQSGPG LVQPSQSLSI TCTVSGFSLT NYGVHWVRQS PGKGLEWLGV IWSGGNTDYN
TPFTSRLSIN KDNSKSQVFF KMNSLQSNDT AIYYCARALT YYDYEFAYWG QGTLVTVSAA
STKGPSVFPL APSSKSTSGG TAALGCLVKD YFPEPVTVSW NSGALTSGVH TFPAVLQSSG
LYSLSSVVTV PSSSLGTQTY ICNVNHKPSN TKVDKKVEPK SCDKTHTCPP CPAPELLGGP
SVFLFPPKPK DTLMISRTPE VTCVVVDVSH EDPEVKFNWY VDGVEVHNAK TKPREEQYNS
TYRVVSVLTV LHQDWLNGKE YKCKVSNKAL PAPIEKTISK AKGQPREPQV YTLPPSRDEL
TKNQVSLTCL VKGFYPSDIA VEWESNGQPE NNYKTTPPVL DSDGSFFLYS KLTVDKSRWQ
QGNVFSCSVM HEALHNHYTQ KSLSLSPGK
panitumumab light chain
SEQ ID NO: 90
DIQMTQSPSS LSASVGDRVT ITCQASQDIS NYLNWYQQKP GKAPKLLIYD ASNLETGVPS
RFSGSGSGTD FTFTISSLQP EDIATYFCQH FDHLPLAFGG GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
panitumumab heavy chain
SEQ ID NO: 91
GHIYYSGNTN YNPSLKSRLT ISIDTSKTQF SLKLSSVTAA DTAIYYCVRD RVTGAFDIWG
QGTMVTVSSA STKGPSVFPL APCSRSTSES TAALGCLVKD YFPEPVTVSW NSGALTSGVH
TFPAVLQSSG LYSLSSVVTV PSSNFGTQTY TCNVDHKPSN TKVDERKCCV ECPAGPSVFL
FPPKPKDTLM ISRTPEVTCV VVDVSHEDPE VQFNWYVDGV EVHNAKTKPR EEQFNSTFRV
VSVLTVVHQD WLNGKEYKCK VSNKGLPAPI EKTISKTKGQ PREPQVYTLP PSREEMTKNQ
VSLTCLVKGF YPSDIAVEWE SNGQPENNYK TTPPMLDSDG SFFLYSKLTV DKSRWQQGNV
FSCSVMHEAL HNHYTQKSLS LSPGK
anti-cMET antibody light chain
SEQ ID NO: 92
DIVMTQAAPS VPVTPGESVS ISCRSSKSLL HSNGNTYLYW FLQRPGQSPQ VLIYRMSNLA
SGVPDRFSGS GSGTAFTLRI RRVEAEDVGV YYCMQNLEYP FTFGGGTKLE IKRTVAAPSV
FIFPPSDEQL KSGTASVVCL LNNFYPREAK VQWKVDNALQ SGNSQESVTE QDSKDSTYSL
SSILTLSKAD YEKHKVYACE VTHQGLSSPV TKSFNRGEC
anti-cMET antibody heavy chain
SEQ ID NO: 93
QVQLQQSGPE LVKSGASVKM SCKASGNTLK DDHVHWVKQR PGQGLEWIGW IYPGGGRTRY
NEKFKGKTTL TADKPSSTVN MLLSSLTSED SAIYFCTNLV FDVWGAGTTV TVSSASTKGP
SVFPLAPSSK STSGGTAALG CLVKDYFPEP VTVSWNSGAL TSGVHTFPAV LQSSGLYSLS
SVVTVPSSSL GTQTYICNVN HKPSNTKVDK KVEPKSCDKT HTCPPCPAPE LLGGPSVFLF
PPKPKDTLMI SRTPEVTCVV VDVSHEDPEV KFNWYVDGVE VHNAKTKPRE EQYNSTYRVV
SVLTVLHQDW LNGKEYKCKV SNKALPAPIE KTISKAKGQP REPQVYTLPP SREEMTKNQV
SLTCLVKGFY PSDIAVEWES NGQPENNYKT TPPVLDSDGS FFLYSKLTVD KSRWQQGNVF
SCSVMHEALH NHYTQKSLSL SPGK
anti-GPC3 antibody light chain
SEQ ID NO: 94
DVVMTQSPLS LPVTPGEPAS ISCRSSQSLV HSNANTYLHW YLQKPGQSPQ LLIYKVSNRF
SGVPDRFSGS GSGTDFTLKI SRVEAEDVGV YYCSQNTHVP PTFGQGTKLE IKRTVAAPSV
FIFPPSDEQL KSGTASVVCL LNNFYPREAK VQWKVDNALQ SGNSQESVTE QDSKDSTYSL
SSILTLSKAD YEKHKVYACE VTHQGLSSPV TKSFNRGEC
anti-GPC3 antibody heavy chain
SEQ ID NO: 95
QVQLVQSGAE VKKPGASVKV SCKASGYTFT DYEMHWVRQA PGQGLEWMGA LDPKTGDTAY
SQKFKGRVTL TADKSTSTAY MELSSLTSED TAVYYCTRFY SYTYWGQGTL VTVSSASTKG
PSVFPLAPSS KSTSGGTAAL GCLVKDYFPE PVTVSWNSGA LTSGVHTFPA VLQSSGLYSL
SSVVTVPSSS LGTQTYICNV NHKPSNTKVD KKVEPKSCDK THTCPPCPAP ELLGGPSVFL
FPPKPKDTLM ISRTPEVTCV VVDVSHEDPE VKFNWYVDGV EVHNAKTKPR EEQYNSTYRV
VSVLTVLHQD WLNGKEYKCK VSNKALPAPI EKTISKAKGQ PREPQVYTLP PSREEMTKNQ
VSLTCLVKGF YPSDIAVEWE SNGQPENNYK TTPPVLDSDG SFFLYSKLTV DKSRWQQGNV
FSCSVMHEAL HNHYTQKSLS LSPGK
Humanized H8 anti-5T4 version 1 VH (protein sequence)
SEQ ID NO: 96
QVQLVQSGAE VKKPGASVKV SCKASGYSFT GYYMHWVKQS PGQGLEWIGR INPNNGVTLY
NQKFKDRVTM TRDTSISTAY MELSRLRSDD TAVYYCARST MITNYVMDYW GQGTLWTVSS
Humanized H8 anti-5T4 VH version 2 (protein sequence)
SEQ ID NO: 97
QVQLVQSGAE VKKPGASVKV SCKASGYSFT GYYMHWVRQA PGQGLEWMGR INPNNGVTLY
NQKFKDRVTM TRDTSISTAY MELSRLRSDD TAVYYCARST MITNYVMDYW GQGTLVTVSS
Humanized H8 anti-5T4 version 1 VL (protein sequence)
SEQ ID NO: 98
DIVMTQSPDS LAVSLGERAT INCKASQSVS NDVAWYQQKP GQSPKLLISY TSSRYAGVPD
RFSGSGSGTD FTLTISSLQA EDVAVYFCQQ DYNSPPTFGG GTKLEIK
Humanized H8 anti-5T4 VL version 2 (protein sequence)
SEQ ID NO: 99
DIVMTQSPDS LAVSLGERAT INCKASQSVS NDVAWYQQKP GQPPKLLIYY TSSRYAGVPD
RFSGSGSGTD FTLTISSLQA EDVAVYYCQQ DYNSPPTFGG GTKLEIK
Anti-IL-15 antibody 146B7 HC CDR1 (protein sequence)
SEQ ID NO: 100
TYWIG
Anti-IL-15 antibody 146B7 HC CDR2 (protein sequence)
SEQ ID NO: 101
IIYPGDSDTR YSPSFQG
Anti-IL-15 antibody 146B7 HC CDR3 (protein sequence)
SEQ ID NO: 102
GNWNCFDY
Anti-IL-15 antibody 146B7 LC CDR1 (protein sequence)
SEQ ID NO: 103
RASQSVSSSY LA
Anti-IL-15 antibody 146B7 LC CDR2 (protein sequence)
SEQ ID NO: 104
GASRRAT
Anti-IL-15 antibody 146B7 LC CDR3 (protein sequence)
SEQ ID NO: 105
QRYGSSHT
Anti-IL-15 antibody 146B7 HC CDR3 ver2 (protein sequence)
SEQ ID NO: 106
GNWNSFDY
Anti-IL-15 antibody 146B7 HC variable domain (protein sequence)
SEQ ID NO: 107
EVQLVQSGAE VKKPGESLKI SCKVSGYFFT TYWIGWVRQM PGKGLEYMGI IYPGDSDTRY
SPSFQGQVTI SADKSISTAY LQWSSLKASD TAMYYCARGG NWNCFDYWGQ GTLVTVSS
Anti-IL-15 antibody 146B7 LC variable domain (protein sequence)
SEQ ID NO: 108
EIVLTQSPGT LSLSPGREAT LSCRASQSVS SSYLAWYQQK PGQAPRLLIY GASRRATGIP
DRFSGSGSGT DFTLTISRLE PEDFAVYYCQ RYGSSHTFGQ GTKLEISRTV AAPSVFIFP
anti-IL-15 scFv1
SEQ ID NO: 109
EVQLVQSGAE VKKPGESLKI SCKVSGYFFT TYWIGWVRQM PGKGLEYMGI IYPGDSDTRY
SPSFQGQVTI SADKSISTAY LQWSSLKASD TAMYYCARGG NWNCFDYWGQ GTLVTVSSGG
GGSGGGGSGG GGSEIVLTQS PGTLSLSPGR EATLSCRASQ SVSSSYLAWY QQKPGQAPRL
LIYGASRRAT GIPDRFSGSG SGTDFTLTIS RLEPEDFAVY YCQRYGSSHT FGQGTKLEIS R
anti-IL-15 scFv2
SEQ ID NO: 110
EIVLTQSPGT LSLSPGREAT LSCRASQSVS SSYLAWYQQK PGQAPRLLIY GASRRATGIP
DRFSGSGSGT DFTLTISRLE PEDFAVYYCQ RYGSSHTFGQ GTKLEISRGG GGSGGGGSGG
GGSEVQLVQS GAEVKKPGES LKISCKVSGY FFTTYWIGWV RQMPGKGLEY MGIIYPGDSD
TRYSPSFQGQ VTISADKSIS TAYLQWSSLK ASDTAMYYCA RGGNWNCFDY WGQGTLVTVS S
IgG1 Fc-hole-Hv-Lv
ASKGD111_CX5_101_1
SEQ ID NO: 111

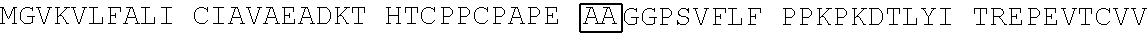

VDVSHEDPEV KFNWYVDGVE VHNAKTKPRE EQYNSTYRVV SVLTVLHQDW LNGKEYKCKV

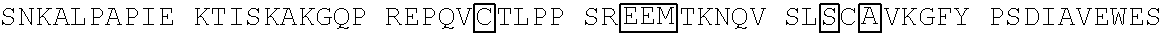

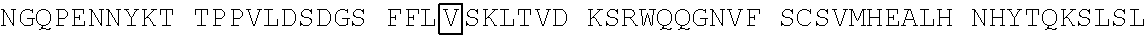

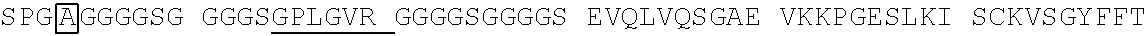

TYWIGWVRQM PGKGLEYMGI IYPGDSDTRY SPSFQGQVTI SADKSISTAY LQWSSLKASD
TAMYYCARGG NWNCFDYWGQ GTLVTVSSGG GGSGGGGSGG GGSGIVLTQS PGTLSLSPGE
RATLSCRASQ SVSSSYLAWY QQKPGQAPRL LIYGASRRAT GIPDRFSGSG SGTDFTLTIS
RLEPEDFAVY YCQRYGSSHT FGQGTKLEIS**
IgG1 Fc-hole-Lv-Hv
ASKGD111_CX5_101_2
SEQ ID NO: 112

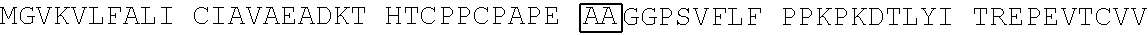

VDVSHEDPEV KFNWYVDGVE VHNAKTKPRE EQYNSTYRVV SVLTVLHQDW LNGKEYKCKV

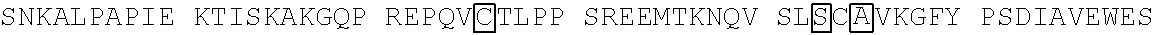

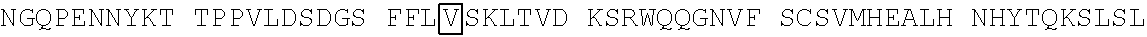

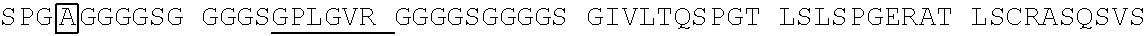

SSYLAWYQQK PGQAPRLLIY GASRRATGIP DRFSGSGSGT DFTLTISRLE PEDFAVYYCQ
RYGSSHTFGQ GTKLEISGGG GSGGGGSGGG GSEVQLVQSG AEVKKPGESL KISCKVSGYF
FTTYWIGWVR QMPGKGLEYM GIIYPGDSDT RYSPSFQGQV TISADKSIST AYLQWSSLKA
SDTAMYYCAR GGNWNCFDYW GQGTLVTVSS **
Fc-hole Fc-hole IgG1, LALA mutation-IL2Rbeta-gamma
ASKGD111_CX5_105_1
SEQ ID NO: 113

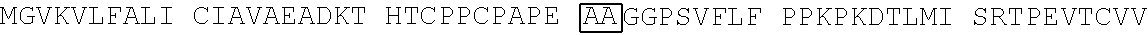

VDVSHEDPEV KFNWYVDGVE VHNAKTKPRE EQYNSTYRVV SVLTVLHQDW LNGKEYKCKV

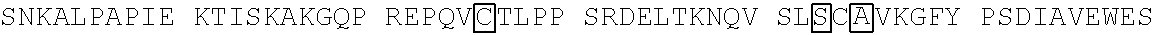

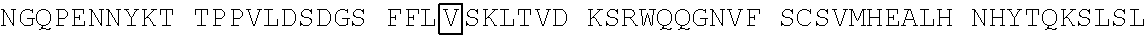

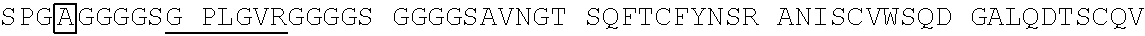

HAWPDRRRWN QTCELLPVSQ ASWACNLILG APDSQKLTTV DIVTLRVLCR EGVRWRVMAI
QDFKPFENLR LMAPISLQVV HVETHRCNIS WEISQASHYF ERHLEFEART LSPGHTWEEA
PLLTLKQKQE WICLETLTPD TQYEFQVRVK PLQGEFTTWS PWSQPLAFRT KPAALGKDTG
GGGSGGGGSG GGGSGGGGSG GGGSGGGGSG GGGSGGGGSP LPEVQCFVFN VEYMNCTWNS
SSEPQPTNLT LHYWYKNSDN DKVQKCSHYL FSEEITSGCQ LQKKEIHLYQ TFVVQLQDPR
EPRRQATQML KLQNLVIPWA PENLTLHKLS ESQLELNWNN RFLNHCLEHL VQYRTDWDHS
WTEQSVDYRH KFSLPSVDGQ KRYTFRVRSR FNPLCGSAQH WSEWSHPIHW**
Fc-hole IgG1, LALA mutation-IL2R-gamma-beta
ASKGD111_CX5_105_2
SEQ ID NO: 114

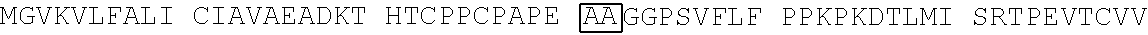

VDVSHEDPEV KFNWYVDGVE VHNAKTKPRE EQYNSTYRVV SVLTVLHQDW LNGKEYKCKV

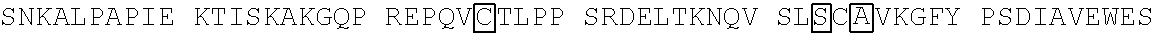

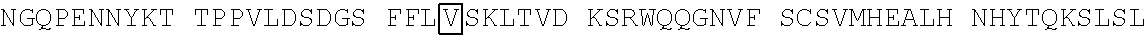

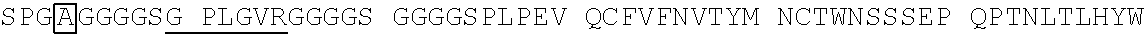

YKNSDNDKVQ KCSHYLFSEE ITSGCQLQKK EIHLYQTFVV QLQDPREPRR QATQMLKLQN
LVIPWAPENL TLHKLSESQL ELNWNNRFLN HCLEHLVQYR TDWDHSWTEQ SVDYRHKFSL
PSVDGQKRYT FRVRSRFNPL CGSAQHWSEW SHPIHWGGGG SGGGGSGGGG SGGGGSGGGG
SGGGGSGGGG SGGGGSAVNG TSQFTCFYNS RANISCVWSQ DGALQDTSCQ VHAWPDRRRW
NQTCELLPVS QASWACNLIL GAPDSQKLTT VDIVTLRVLC REGVRWRVMA IQDFKPFENL
RLMAPISLQV VHVETHRCNI SWEISQASHY FERHLEFEAR TLSPGHTWEE APLLTLKQKQ
EWICLETLTP DTQYEFQVRV KPLQGEFTTW SPWSQPLAFR TKPAALGKDT**
Fc-hole Fc-hole IgG1, LALA mutation-IL2R-beta-Ctergamma
ASKGD111_CX5_105_3
SEQ ID NO: 115

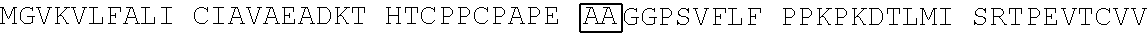

VDVSHEDPEV KFNWYVDGVE VHNAKTKPRE EQYNSTYRVV SVLTVLHQDW LNGKEYKCKV

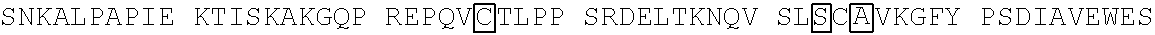

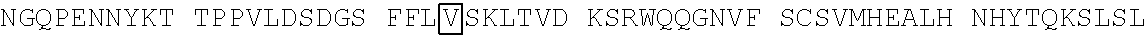

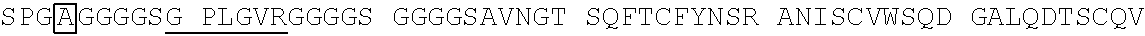

HAWPDRRRWN QTCELLPVSQ ASWACNLILG APDSQKLTTV DIVTLRVLCR EGVRWRVMAI
QDFKPFENLR LMAPISLQVV HVETHRCNIS WEISQASHYF ERHLEFEART LSPGHTWEEA
PLLTLKQKQE WICLETLTPD TQYEFQVRVK PLQGEFTTWS PWSQPLAFRT KPAALGKDTG
GGGSGGGGSG GGGSGGGGSG GGGSGGGGSG GGGSGGGGSA PENLTLHKLS ESQLELNWNN
RFLNHCLEHL VQYRTDWDHS WTEQSVDYRH KFSLPSVDGQ KRYTFRVRSR FNPLCGSAQH
WSEWSHPIHW **
Fc-knob-Sushi-IL-15 (Q108E) IgG1_allotype EEM, LALA
mutation H435R/Y436F
5′XbaI,3′PmeI
ASKGD111_CX5_74_3
SEQ ID NO: 116

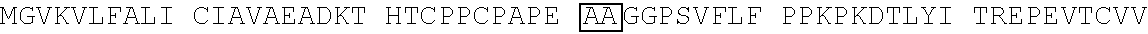

VDVSHEDPEV KFNWYVDGVE VHNAKTKPRE EQYNSTYRVV SVLTVLHQDW LNGKEYKCKV

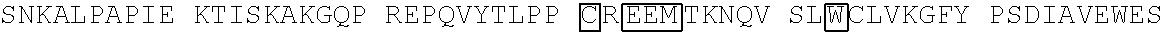

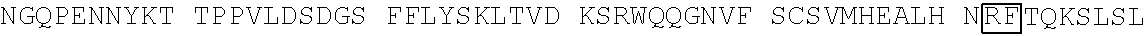

SPGKGGGGSG GGGSGGGGSI TCPPPMSVEH ADIWVKSYSL YSRERYICNS GFKRKAGISS
LTECVLNKAT NVAHWTTPSL KCIRGGSGGG GSGGGSGGGG SNWVNVISDL KKIEDLIQSM
HIDATLYTES DVHPSCKVTA MKCFLLELQV ISLESGDASI HDTVENLIIL ANNSLSSNGN

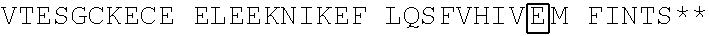

Fc-hole IgG1, LALA mutation-IL2Rbeta D68E, not
cleavable
ASKGD111_CX5_76_2
SEQ ID NO: 117

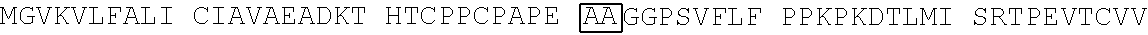

VDVSHEDPEV KFNWYVDGVE VHNAKTKPRE EQYNSTYRVV SVLTVLHQDW LNGKEYKCKV

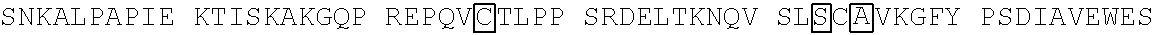

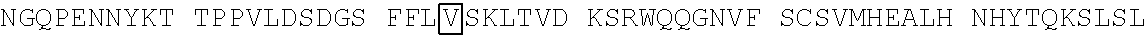

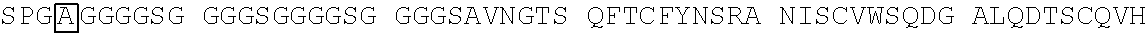

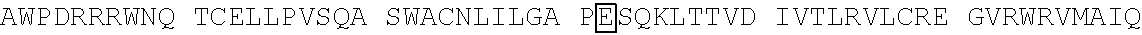

DFKPFENLRL MAPISLQVVH VETHRCNISW EISQASHYFE RHLEFEARTL SPGHTWEEAP
LLTLKQKQEW ICLETLTPDT QYEFQVRVKP LQGEFTTWSP WSQPLAFRTK PAALGKDT
Fc-Sushi-IL-15vN65D IgG1_allotype EEM, LALA mutation, YTE.
ASKD215_CX7.40.1
SEQ ID NO: 118

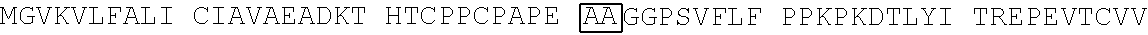

VDVSHEDPEV KFNWYVDGVE VHNAKTKPRE EQYNSTYRVV SVLTVLHQDW LNGKEYKCKV

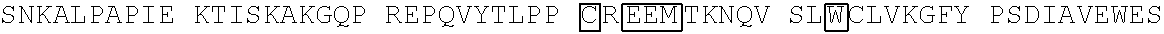

NGQPENNYKT TPPVLDSDGS FFLYSKLTVD KSRWQQGNVF SCSVMHEALH NHYTQKSLSL

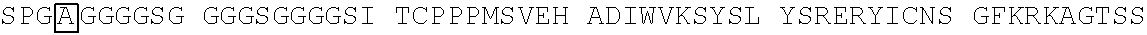

LTECVLNKAT NVAHWTTPSL KCIRGGSGGG GSGGGSGGGG SNWVNVISDL KKIEDLIQSM

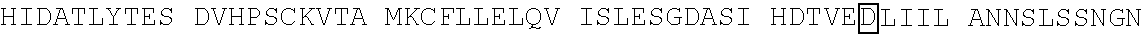

VTESGCKECE ELEEKNIKEF LQSFVHIVQM FINTS
IgG1 Fc-hole-MMP/matriptase-VL-VH
ASKD215_CX7_40_2
SEQ ID NO: 119

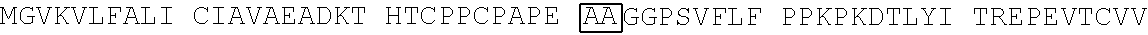

VDVSHEDPEV KFNWYVDGVE VHNAKTKPRE EQYNSTYRVV SVLTVLHQDW LNGKEYKCKV

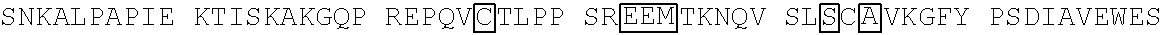

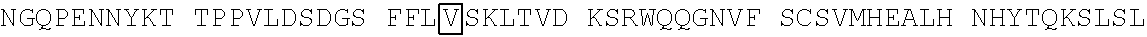

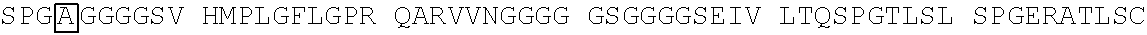

RASQSVSSSY LAWYQQKPGQ APRLLIYGAS RRATGIPDRF SGSGSGTDFT LTISRLEPED
FAVYYCQRYG SSHTFGQGTK LEISGGGGSG GGGSGGGGSE VQLVQSGAEV KKPGESLKIS
CKVSGYFFTT YWIGWVRQMP GKGLEYMGII YPGDSDTRYS PSFQGQVTIS ADKSISTAYL
QWSSLKASDT AMYYCARGGN WNCFDYWGQG TLVTVSS
IgG1 Fc-hole-MMP/matriptase-VL-VH with the 2nd cleavage
between VL and VH
ASKD215_CX7_40_3
SEQ ID NO: 120

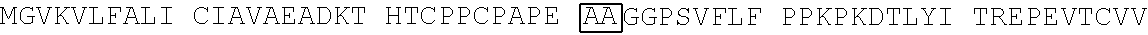

VDVSHEDPEV KFNWYVDGVE VHNAKTKPRE EQYNSTYRVV SVLTVLHQDW LNGKEYKCKV

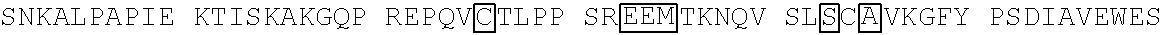

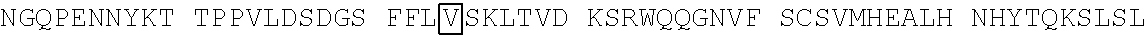

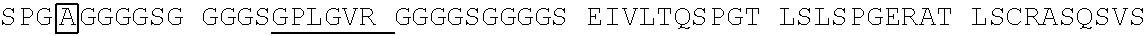

SSYLAWYQQK PGQAPRLLIY GASRRATGIP DRFSGSGSGT DFTLTISRLE PEDFAVYYCQ
RYGSSHTFGQ GTKLEISGGG GSGGGGSRQA RVVNGGGGGS EVQLVQSGAE VKKPGESLKI
SCKVSGYFFT TYWIGWVRQM PGKGLEYMGI IYPGDSDTRY SPSFQGQVTI SADKSISTAY
LQWSSLKASD TAMYYCARGG NWNCFDYWGQ GTLVTVSS
Fc-IL-15vN65D, Knob chain, without Sushi
ASKD215_CX7.56.2,
SEQ ID NO: 121

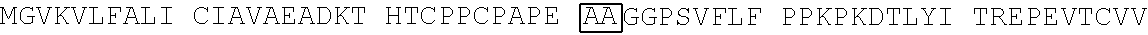

VDVSHEDPEV KFNWYVDGVE VHNAKTKPRE EQYNSTYRVV SVLTVLHQDW LNGKEYKCKV

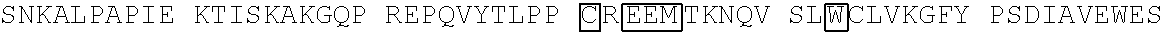

NGQPENNYKT TPPVLDSDGS FFLYSKLTVD KSRWQQGNVF SCSVMHEALH NHYTQKSLSL

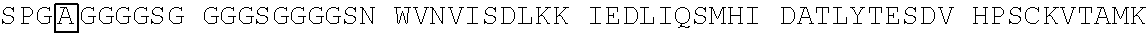

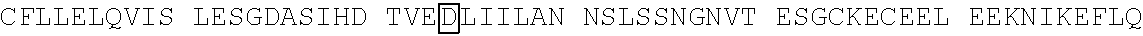

SFVHIVQMFI NTS
Fc knob chain with longer linker between Sushi and IL-15v
ASKD215_CX7_56_3
SEQ ID NO: 122

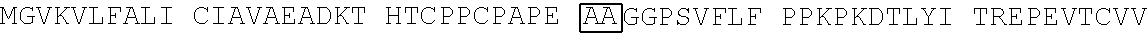

VDVSHEDPEV KFNWYVDGVE VHNAKTKPRE EQYNSTYRVV SVLTVLHQDW LNGKEYKCKV

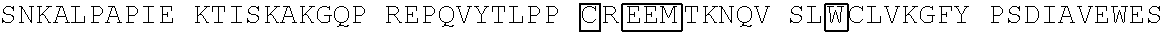

NGQPENNYKT TPPVLDSDGS FFLYSKLTVD KSRWQQGNVF SCSVMHEALH NHYTQKSLSL

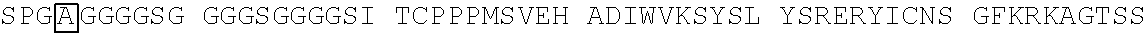

LTECVLNKAT NVAHWTTPSL KCIRGGGGSG GGSGGGGSAA GGGGSGGGGS GGGGSNWVNV

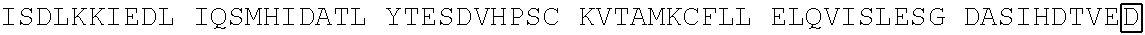

LIILANNSLS SNGNVTESGC KECEELEEKN IKEFLQSFVH IVQMFINTS
Anti-IL-15 antibody 146B7 LC variable domain (protein sequence) ver2
SEQ ID NO: 123
EIVLTQSPGT LSLSPGREAT LSCRASQSVS SSYLAWYQQK PGQAPRLLIY GASRRATGIP
DRFSGSGSGT DFTLTISRLE PEDFAVYYCQ RYGSSHTFGQ GTKLE
anti-IL-15 scFv1 ver2
SEQ ID NO: 124
EVQLVQSGAE VKKPGESLKI SCKVSGYFFT TYWIGWVRQM PGKGLEYMGI IYPGDSDTRY
SPSFQGQVTI SADKSISTAY LQWSSLKASD TAMYYCARGG NWNCFDYWGQ GTLVTVSSGG
GGSGGGGSGG GGSEIVLTQS PGTLSLSPGR EATLSCRASQ SVSSSYLAWY QQKPGQAPRL
LIYGASRRAT GIPDRFSGSG SGTDFTLTIS RLEPEDFAVY YCQRYGSSHT FGQGTKLE
anti-Trop-2 antibody light chain CDR1
SEQ ID NO: 125
KASQDVSIAV A
anti-Trop-2 antibody light chain CDR2
SEQ ID NO: 126
SASYRYT
anti-Trop-2 antibody light chain CDR3
SEQ ID NO: 127
QQHYITPLT
anti-Trop-2 antibody heavy chain CDR1
SEQ ID NO: 128
NYGMN
anti-Trop-2 antibody heavy chain CDR2
SEQ ID NO: 129
WINTYTGEPT YTDDFKG
anti-Trop-2 antibody heavy chain CDR3
SEQ ID NO: 130
GGFGSSYWY FDV
anti-mesothelin antibody light chain CDR1
SEQ ID NO: 131
SASSSVSYM H
anti-mesothelin antibody light chain CDR2
SEQ ID NO: 132
DTSKLAS
anti-mesothelin antibody light chain CDR3
SEQ ID NO: 133
QQWSGY PLT
anti-mesothelin antibody heavy chain CDR1
SEQ ID NO: 134
GYTMN
anti-mesothelin antibody heavy chain CDR2
SEQ ID NO: 135
LITPYNGASS YNQKFRG
anti-mesothelin antibody heavy chain CDR3
SEQ ID NO: 136
GGYDGRGFDY
Homo sapiens interleukin 15 receptor subunit alpha (IL-
15Rα), transcript variant 1, mRNA
SEQ ID NO: 137
ctgggcagcg ctcgcccggg gagtccagcg gtgtcctgtg gagctgccgc catggccccg
cggcgggcgc gcggctgccg gaccctcggt ctcccggcgc tgctactgct gctgctgctc
cggccgccgg cgacgcgggg catcacgtgc cctcccccca tgtccgtgga acacgcagac
atctgggtca agagctacag cttgtactcc agggagcggt acatttgtaa ctctggtttc
aagcgtaaag ccggcacgtc cagcctgacg gagtgcgtgt tgaacaaggc cacgaatgtc
gcccactgga caacccccag tctcaaatgc attagagacc ctgccctggt tcaccaaagg
ccagcgccac cctccacagt aacgacggca ggggtgaccc cacagccaga gagcctctcc
ccttctggaa aagagcccgc agcttcatct cccagctcaa acaacacagc ggccacaaca
gcagctattg tcccgggctc ccagctgatg ccttcaaaat caccttccac aggaaccaca
gagataagca gtcatgagtc ctcccacggc accccctctc agacaacagc caagaactgg
gaactcacag catccgcctc ccaccagccg ccaggtgtgt atccacaggg ccacagcgac
accactgtgg ctatctccac gtccactgtc ctgctgtgtg ggctgagcgc tgtgtctctc
ctggcatgct acctcaagtc aaggcaaact cccccgctgg ccagcgttga aatggaagcc
atggaggctc tgccggtgac ttgggggacc agcagcagag atgaagactt ggaaaactgc
tctcaccacc tatgaaactc ggggaaacca gcccagctaa gtccggagtg aaggagcctc
tctgctttag ctaaagacga ctgagaagag gtgcaaggaa gcgggctcca ggagcaagct
caccaggcct ctcagaagtc ccagcaggat ctcacggact gccgggtcgg cgcctcctgc
gcgagggagc aggttctccg cattcccatg ggcaccacct gcctgcctgt cgtgccttgg
acccagggcc cagcttccca ggagagacca aaggcttctg agcaggattt ttatttcatt
acagtgtgag ctgcctggaa tacatgtggt aatgaaataa aaaccctgcc ccgaatcttc
cgtccctcat cctaactttc agttcacaga gaaaagtgac atacccaaag ctctctgtca
attacaaggc ttctcctggc gtgggagacg tctacaggga agacaccagc gtttgggctt
ctaaccaccc tgtctccagc tgctctgcac acatggacag ggacctggga aaggtgggag
agatgctgag cccagcgaat cctctccatt gaaggattca ggaagaagaa aactcaactc
agtgccattt tacgaatata tgcgtttata tttatacttc cttgtctatt atatctatac
attatatatt atttgtattt tgacattgta ccttgtataa acaaaataaa acatctattt
tcaata
noncleavable peptide linker
SEQ ID NO: 138
GSAGSAAGSG EF
noncleavable peptide linker, wherein n1 = 1, 2, or 3,
and n2 = 1, 2, or 3.
SEQ ID NO: 139
(GGGGS)n₁GSAGSAAGSGEF(GGGGS)n₂
noncleavable peptide linker
SEQ ID NO: 140
(GGGGS)n₁AA(GGGGS)n₂; wherein n1 = 2 or 3, and n2 = 2 or 3.

1. A prodrug comprising an IL-15 cytokine moiety (A), a masking moiety (M), a carrier moiety (C), and a Sushi domain (S), wherein
the masking moiety binds to the IL-15 cytokine moiety and inhibits a biological activity of the IL-15 cytokine moiety,
the masking moiety is fused to the carrier moiety,
the Sushi domain is fused to the carrier moiety, and
the IL-15 cytokine moiety is fused to the Sushi domain.

2. The prodrug of claim 1, wherein
the masking moiety is fused to the carrier moiety through a first peptide linker,
the Sushi domain is fused to the carrier moiety through a second peptide linker, and
the IL-15 cytokine moiety is fused to the Sushi domain through a third peptide linker, and wherein at least one of the three peptide linkers is cleavable.

3. The prodrug of claim 2, wherein the third peptide linker is at least 15, 20, 25, or 30 amino acids in length, optionally wherein the third peptide linker comprises SEQ ID NO: 139 or 140.

4. A prodrug comprising an IL-15 cytokine moiety (A), a masking moiety (M), a carrier moiety (C), and a Sushi domain (S), wherein
the masking moiety binds to the IL-15 cytokine moiety and inhibits a biological activity of the IL-15 cytokine moiety,
the IL-15 cytokine moiety is fused to the carrier moiety,
the Sushi domain is fused to the carrier moiety, and
the masking moiety is fused to the Sushi domain.

5. The prodrug of claim 4, wherein
the IL-15 cytokine moiety is fused to the carrier moiety through a first peptide linker,
the Sushi domain is fused to the carrier moiety through a second peptide linker, and
the masking moiety is fused to the Sushi domain through a third peptide linker, and optionally wherein at least one of the three peptide linkers is cleavable.

6. The prodrug of any one of claims 1-5, wherein the masking moiety comprises an extracellular domain (ECD) of a receptor of the IL-15 cytokine moiety.

7. The prodrug of claim 6, wherein the masking moiety comprises an ECD of human IL-2Rβ or a functional analog thereof, and/or an ECD of human IL-2Rγ or a functional analog thereof.

8. The prodrug of claim 7, wherein the ECD of human IL-2Rγ or a functional analog thereof comprises SEQ ID NO: 6, or an amino acid sequence at least 90% identical thereto.

9. The prodrug of claim 7, wherein the ECD of human IL-2Rβ or a functional analog thereof comprises SEQ ID NO: 3, 4, or 5, or an amino acid sequence at least 90% thereto.

10. The prodrug of any one of claims 1-5, wherein the masking moiety comprises an antibody fragment that binds to the IL-15 cytokine moiety.

11. A prodrug comprising an IL-15 cytokine moiety (A), a masking moiety (M), a carrier moiety (C), and optionally a Sushi domain (S), wherein
the masking moiety comprises an antibody fragment that binds to the IL-15 cytokine moiety and inhibits a biological activity of the IL-15 cytokine moiety, and
the masking moiety is fused to the carrier moiety, to the IL-15 cytokine moiety, or to the Sushi domain through a peptide linker.

12. The prodrug of claim 10 or 11, wherein the antibody fragment is an ScFv or Fab comprising heavy chain CDR1-3 and light chain CDR1-3 of an anti-IL-15 antibody selected from 146B7, 146H5, 404E4, and 404A8.

13. The prodrug of claim 10 or 11, wherein the antibody fragment comprises heavy chain CDR (HCDR) 1 comprising SEQ ID NO: 100, HCDR2 comprising SEQ ID NO: 101, HCDR3 comprising SEQ ID NO: 102 or 106, light chain CDR (LCDR) 1 comprising SEQ ID NO: 103, LCDR2 comprising SEQ ID NO: 104, and LCDR3 comprising SEQ ID NO: 105.

14. The prodrug of claim 10 or 11, wherein the antibody fragment comprises (i) a heavy chain variable domain comprising SEQ ID NO: 107 or an amino acid sequence at least 95% identical thereto, and a light chain variable domain comprising SEQ ID NO: 108 or 123 or an amino acid sequence at least 95% identical thereto; (ii) SEQ ID NO: 109; (iii) SEQ ID NO: 110; or (iv) SEQ ID NO: 124.

15. The prodrug of claim 13 or 14, wherein the Cys residue of the heavy chain CDR3 is mutated to Ser, Thr, Met, Ala, Gly, Asn or Gln.

16. The prodrug of any one of the preceding claims, wherein the masking moiety does not interfere with or has minimum impact on the binding of the IL-15 cytokine moiety to IL-15Rα.

17. The prodrug of any one of the preceding claims, wherein the IL-15 cytokine moiety is a human IL-15 polypeptide comprising SEQ ID NO: 2 or a mutein thereof.

18. The prodrug of claim 17, wherein the human IL-15 polypeptide comprises one or more mutations selected from N1A, N1D, N4A, N4D, I6T, S7A, DBA, DBT, D8E, D8N, K10A, K10D, K11A, K11D, E46, V49, L45, S51, L52, D61A, D61N, T62L, T62A, E64A, E64L, E64K, E64Q, N65A, N65L, N65D, L66D, L66E, I 67D, 167E, I68S, 168E, L69S, L69E, N72A, N72D, V63E, V63D, L66E, L66D, 167E, I67D, Q108E, N112A, N1D/D61N, N1D/E64Q, N4D/D61N, N4D/E64Q, D8N/D61N, D8N/E64Q, D61N/E64Q, E64Q/Q108E, N1D/N4D/D8N, D61N/E64Q/N65D, N1D/D61N/E64Q, N1D/Q108E, N1D/D61N/E64Q/Q108E, N4D/D61N/E64Q/Q108E, and D3ON/E64Q/N65D relative to SEQ ID NO: 2.

19. The prodrug of any one of the preceding claims, wherein the carrier moiety is a PEG molecule, an albumin, an albumin fragment, an antibody Fc domain, or an antibody or an antigen-binding fragment thereof.

20. The prodrug of claim 19, wherein the carrier moiety is an antibody Fc domain or an antibody comprising mutations L234A and L235A (“LALA”) (EU numbering).

21. The prodrug of claim 19 or 20, wherein the carrier moiety is an antibody Fc domain or an antibody comprising knobs-into-holes mutations, and wherein the IL-15 cytokine moiety and the masking moiety are fused to different polypeptide chains of the antibody Fc domain or to the different heavy chains of the antibody.

22. The prodrug of claim 21, wherein
the knobs-into-holes mutations comprise a T366Y “knob” mutation on a polypeptide chain of the Fc domain or a heavy chain of the antibody, and a Y407T “hole” mutation in the other polypeptide of the Fc domain or the other heavy chain of the antibody, or
the knobs-into-holes mutations comprise Y349C and/or T366W mutations in the CH3 domain of the “knob chain” and E356C, T366S, L368A, and/or Y407V mutations in the CH3 domain of the “hole chain” (EU numbering).

23. The prodrug of claim 19, wherein the carrier moiety is an IgG₄ Fc domain, and wherein said first polypeptide comprises an amino acid sequence at least 99% identical as one shown in SEQ ID NOs: 80, 81 or 87, and said second polypeptide chain comprises an amino acid sequence at least 99% identical as one selected from SEQ ID NOs: 82-86.

24. The prodrug of claim 19 or 20, wherein the carrier moiety is an anti-PD-1 antibody comprising a light chain having an amino acid sequence at least 99% identical to SEQ ID NO: 55 or 56; a first heavy chain having an amino acid sequence at least 99% identical to SEQ ID NO: 54, 60, or 61; and a second heavy chain having an amino acid sequence at least 99% identical to SEQ ID NO: 52, 53, 58, 59, 62, 63, or 69.

25. The prodrug of claim 19 or 20, wherein the carrier moiety is an anti-PD-1 antibody comprising a light chain having an amino acid sequence at least 99% identical to SEQ ID NO: 55; a first heavy chain having an amino acid sequence at least 99% identical to SEQ ID NO: 66; and a second heavy chain having an amino acid sequence at least 99% identical to SEQ ID NO: 64, 65, 67, or 68.

26. The prodrug of claim 19 or 20, wherein the carrier moiety is an anti-PD-Ll antibody comprising a light chain having an amino acid sequence at least 99% identical to SEQ ID NO: 50 or 51; a first heavy chain having an amino acid at least 99% identical to SEQ ID NO: 47, 48 or 49; and a second heavy chain having an amino acid sequence at least 99% identical to SEQ ID NO: 45 or 46.

27. The prodrug of claim 19 or 20, wherein the carrier moiety is an antibody or an antigen-binding fragment thereof that specifically binds to one or more antigens selected from PD-1, PD-L1, CTLA-4, LAG-3, TIM-3, CD47, and TIGIT.

28. The prodrug of any one of claims 19-27, wherein the carrier moiety is an antibody Fc domain or an antibody, and the prodrug comprises the following polypeptide pairs (from N-terminus to C-terminus):
a) C1-A and C2-S-M,
b) A-C1 and M-S-C2,
c) C1-S-A and C2-M,
d) C1-A-S and C2-M,
e) S-A-C1 and M-C2, or
f) A-S-C1 and M-C2; and
wherein C1 and C2 are the first and second polypeptide chains, respectively, of the Fc domain, or are the first and second heavy chains, respectively, of the antibody; and “-” is a direct peptidyl bond or a peptide linker.

29. The prodrug of any one of the preceding claims, wherein the Sushi domain comprises SEQ ID NO: 7 or 9, or an amino acid sequence at least 90% identical thereto.

30. The prodrug of any one of the preceding claims, wherein at least one of the first, second, and third peptide linkers is a noncleavable peptide linker, optionally selected from SEQ ID NOs: 11-16.

31. The prodrug of any one of the preceding claims, wherein at least one of the first, second, and third peptide linkers is a cleavable peptide linker comprising a substrate sequence of urokinase-type plasminogen activator (uPA), matriptase, matrix metallopeptidase (MMP) 2, or MMP9.

32. The prodrug of claim 31, wherein the cleavable peptide linker comprises substrate sequences of (i) both uPA and MMP2, (ii) both uPA and MMP9, (iii) uPA, MMP2 and MMP9, or (iv) MMP2 and matriptase.

33. The prodrug of claim 31, wherein the cleavable peptide linker comprises an amino acid sequence selected from SEQ ID NOs: 17-36.

34. The prodrug of any one of the preceding claims, wherein the cleavable peptide linker is cleavable by one or more proteases located at a tumor site or its surrounding environment, and the cleavage leads to activation of the prodrug at the tumor site or surrounding environment.

35. A pharmaceutical composition comprising the prodrug of any one of claims 1-34 and a pharmaceutically acceptable excipient.

36. A polynucleotide or polynucleotides encoding the prodrug of any one of claims 1-34.

37. An expression vector or vectors comprising the polynucleotide or polynucleotides of claim 36.

38. A host cell comprising the vector(s) of claim 37.

39. The host cell of claim 38, wherein the gene(s) encoding uPA, matriptase, MMP-2, and/or MMP-9 are knocked out in the host cell.

40. A method of making the prodrug of any one of claims 1-34, comprising culturing the host cell of claim 38 or 39 under conditions that allow expression of the prodrug, wherein the host cell is a mammalian cell, and isolating the prodrug.

41. A method of treating a cancer or an infectious disease or stimulating the immune system in a patient in need thereof, comprising administering to the patient a therapeutically effective amount of the pharmaceutical composition of claim 35.

42. The method of claim 41, wherein the patient has a viral infection, or a cancer selected from the group consisting of breast cancer, lung cancer, pancreatic cancer, esophageal cancer, medullary thyroid cancer, ovarian cancer, uterine cancer, prostate cancer, testicular cancer, colorectal cancer, and stomach cancer.
